(19) United States
(12) Patent Application Publication
(10) Pub. No.: US 2025/0002873 A1
(43) Pub. Date: Jan. 2, 2025

(54) RETINOID PRODUCTION

(71) Applicant: DSM IP ASSETS B.V., Heerlen (NL)

(72) Inventors: Valmik Kanubhai VYAS, Winchester, MA (US); Peter Louis HOUSTON, Boston, MA (US); Jenna McMAHON, Tewksbury, MA (US); René Marcel DE JONG, Amsterdam (NL)

(21) Appl. No.: 18/700,931

(22) PCT Filed: Oct. 19, 2022
(86) PCT No.: PCT/EP2022/079143

§ 371 Date: Apr. 12, 2024

Related U.S. Application Data
(60) Provisional application No. 63/257,222, filed on Oct. 19, 2021.

(30) Foreign Application Priority Data
Oct. 26, 2021 (EP) 21204854.0

(51) Int. Cl. C12N 9/04; C12P 7/22

(52) U.S. Cl. CPC C12N 9/0006; C12P 7/22; C12Y 101/01105

(57) ABSTRACT

The present invention is related to bio-production of retinoids with improved purity profile, particularly bio-retinoids with a high percentage of retinyl acetate, wherein the flux towards retinol is increased and wherein the percentage of unwanted by-products such as dihydroretinoids or retinal is minimized to a range of below 10% by expressing retinol dehydrogenase (EC 1.1.1.105, RDH22) derived from Yarrowia lipolytica (Candida lipolytica) or Wickerhamomyces anomalus (Hanensula anomala).

The present invention is related to bio-production of retinoids with improved purity profile, particularly bio-retinoids with a high percentage of retinyl acetate, wherein the flux towards retinol is increased and wherein the percentage of unwanted by-products such as dihydroretinoids is minimized to a range of below 10%.

Retinol is an important intermediate/precursor in the process of retinoid production, particularly such as vitamin A production. Retinoids, including vitamin A, are one of very important and indispensable nutrient factors for human beings which have to be supplied via nutrition. Retinoids promote well-being of humans, inter alia in respect of vision, the immune system and growth.

Current chemical production methods for retinoids, including vitamin A and precursors thereof, have some undesirable characteristics such as e.g. high-energy consumption, complicated purification steps and/or undesirable by-products. Therefore, over the past decades, other approaches to manufacture retinoids, including vitamin A and precursors thereof, including microbial conversion steps, which would be more economical as well as ecological, have been investigated.

Unfortunately, the biological systems that produce retinoids are industrially intractable and/or produce the compounds at such low levels that commercial scale isolation is not practical. The most limiting factors include instability of intermediates including instability of retinol, inefficient enzymatic conversions and/or impurity due to accumulation of unwanted side-products.

Thus, it is an ongoing task to look for an improved process for fermentative production of retinoids, particularly with a high content of stable forms such as e.g. retinyl acetate, and at the same time to reduce the formation of unwanted by-products such as e.g. dihydroretinoids.

Surprisingly, we now identified a novel class of retinal reducing enzymes (RDHs) that are involved in formation of retinol, wherein the use of said enzymes in a fermentative production of retinoids leads to about 90% reduction of by-products such as dihydroretinoids and retinal that are generated during said fermentation process.

Thus, the present invention is related to a process for production of a retinoid-mix, such as a fermentation process using a suitable retinoid-producing host cell, said retinoid-mix comprising dihydroretinoids with a percentage of less than about 10% based on total retinoids within said mix.

As used herein, a “retinoid mix” is defined as all retinoids formed during a retinoid production process, such as e.g. fermentation process using a suitable host cell that is expressing suitable enzymes involved in formation of retinoids including but not limited to retinal, retinol, and retinyl acetate and optionally furthermore expressing enzymes involved in formation of beta-carotene. Said retinoid mix comprises retinal, retinol, retinyl acetate, and dihydroretinods.

Particularly, such retinoid-mix according to the present invention comprises dihydroretinoids, retinal, and retinyl acetate, wherein the percentage of dihydroretinoids is less than about 10% based on total retinoids, preferably wherein the percentage of retinal is less than about 8% based on total retinoids, further preferably wherein the percentage of retinyl acetate is at least about 80% based on total retinoids, even further preferably wherein the percentage of trans-isomers within said mix is at least about 80% based on the amount of all cis- and trans-retinoids.

In one embodiment, the percentage of dihydroretinoids based on total retinoids within said retinoid-mix is about less than 10, such as 9, 8, 7, 6, 5, 4, 3, 1% or less, preferably 2% or less, more preferably 1% or less, such as 0.9, 0.8, 0.7, 0.6, 0.5, 0.4, 0.3, 0.2, 0.1% or less, such as in the range of 1% to 0.1, 0.01, or 0.001%, as measurable at the end of the production or fermentation process.

In one embodiment, the percentage of retinal based on total retinoids within said retinoid-mix is about less than 8, such as 7, 6, 5, 4, 3, 1% or less, preferably 2% or less, more preferably 1% or less, such as 0.9, 0.8, 0.7, 0.6, 0.5, 0.4, 0.3, 0.2, 0.1% or less, such as in the range of 1% to 0.1, 0.01, or 0.001%, as measurable at the end of the production or fermentation process.

In a further embodiment, the percentage of retinyl acetate based on total retinoids within said retinoid-mix is at least about 80%, such as e.g. 85, 90, 92, 95, 98% or more retinyl acetate, as measurable at the end of the production or fermentation process using a suitable retinyl-acetate producing host cell.

In another embodiment, the percentage of trans-isoforms based on the total amount of cis and trans-isomers in said retinoid-mix is at least about 80, 85, 90, 92, 95, 98% or more, as measurable at the end of the production or fermentation process. Particularly, said mix comprises at least about 80% of trans-retinyl acetate, i.e. 80% of the retinyl acetate in the retinoid-mix is present in the trans-isomer.

The present invention is related to a biotechnological process for production of a retinoid-mix as defined herein comprising enzymatic conversion of retinal into retinol in the presence of an RDH22 homolog, particularly obtainable from fungi, as defined herein, particularly comprising cultivation of a suitable host cell, more particularly a retinal-producing host cell, expressing said RDH22 homolog under suitable conditions that allow formation of said retinoid-mix as defined herein.

The terms “RDH”, “retinal reductase”, “retinal reducing enzyme”, “enzyme involved in formation of retinol” or “enzyme involved in conversion of retinal into retinol” are used interchangeably herein and refer to enzymes [EC 1.1.1.105] which are involved in the conversion/catalysis of retinal into retinol as well as the back-conversion from retinol to retinal. With regards to RDH22, the formation of dihydroretinoids is reduced to a percentage of less than about 10% detectable in a retinoid-mix formed during a fermentation process using a suitable retinoid-producing host cell and under suitable conditions and the flux towards retinol is increased such that the percentage of retinal present in said retinoid-mix as detectable at the end of the fermentation process is less than about 8% based on total retinoids.

Thus, the present invention is related to RDH22 homologs as defined herein capable of catalyzing retinal into retinol, wherein the conversion towards retinol is increased, leading to a retinoid-mix with a percentage of less than about 8% retinal based on total retinoids, which can be measured at the end of the fermentation process by known methods, particularly process for production of a retinoid-mix with at least about 80% retinyl acetate as described herein.

In one embodiment, the present invention is directed to RDH22 homologs obtainable from Yarrowia, preferably Yarrowia lipolytica, comprising YlRDH22 according to SEQ ID NO:1 and enzymes originated from other source organisms or being artificially constructed (based on digital sequence information) but having activity corresponding to the respective Yarrowia RDH22 according to SEQ ID NO: 1. Using such YlRDH22 homolog as defined herein in a process comprising production of retinol and retinyl acetate, the percentage of retinal accumulated during such production process and present in the retinoid-mix measured at the end of the production process could be reduced to less than 1% based on total retinoids.

Preferably, the present invention is directed to polypeptides with at least about 30, 35, 40, 45, 50, 55, 60, 65, 70, 75, 80, 85, 90, 92, 95, 97, 98, 99% or up to 100% identity to SEQ ID NO:1 including polynucleotides encoding said polypeptides, such as e.g. polynucleotide according to SEQ ID NO:2 or 3 as well as enzymes having equivalent enzymatic activity, i.e. activity corresponding to YlRDH22 as defined above but are originated from another source organism or being based on digital sequence information, particularly being originated from another fungal organism.

In one embodiment, the present invention is directed to RDH22 homologs obtainable from Wickerhamomyces, preferably Wickerhamomyces anomalus, comprising WaRDH22 according to SEQ ID NO:4 and enzymes originated from other source organisms or being artificially constructed (based on digital sequence information) but having activity corresponding to the respective Wickerhamomyces RDH22 according to SEQ ID NO:4. Using such WaRDH22 homolog as defined herein in a process comprising production of retinol and retinyl acetate, the percentage of retinal accumulated during such production process and present in the retinoid-mix measured at the end of the production process could be reduced to about 2% or less based on total retinoids. with a conversion rate of more than 90% towards formation of retinol.

Preferably, the present invention is directed to polypeptides with at least about 30, 35, 40, 45, 50, 55, 60, 65, 70, 75, 80, 85, 90, 92, 95, 97, 98, 99% or up to 100% identity to SEQ ID NO:4 including polynucleotides encoding said polypeptides, such as e.g. polynucleotide according to SEQ ID NO:5 or 6 as well as enzymes having equivalent enzymatic activity, i.e. activity corresponding to WaRDH22 as defined above but are originated from another source organism or being based on digital sequence information, particularly being originated from another fungal organism.

The RDH22 enzymes as defined herein are catalyzing the conversion of retinal into retinol with a conversion being shifted towards production of retinol, wherein the substrate (i.e. retinal) can be either cis-, trans- or a mix of cis-/trans-retinal in any possible ratio, with a percentage of at least about 80% trans-isomers being preferred. Conversion of said retinal-mix with about 80% trans-retinal would lead to retinol and optionally retinyl acetate with about the same ratio of trans to cis-isomers in a respective retinoid-mix obtained by a process for fermentative production as defined herein, such as starting from beta-carotene using a host cell capable of retinal, retinol and/or retinyl-acetate production. The skilled person knows how to generate retinal from beta-carotene using beta-carotene oxygenases (BCOs), such as exemplified in WO2019057999 or WO2021009194. Enzymes, particularly acetyl transferases (ATFs), more preferably ATF1s, host cells and processes for conversion of retinol into retinyl acetate are known, such as e.g. from WO2019058000 or WO2020141168.

The terms “conversion” “enzymatic conversion or “cleavage” in connection with enzymatic catalysis of substrates such as e.g. beta-carotene, retinal or retinol are used interchangeably herein and refer to the action of the specific enzymes, including but not limited to BCOs, RDHs, particularly RDH22, or ATFs as defined herein involved in formation of retinal, retinol or retinyl acetate.

As used herein, the term “dihydroretinoids” and “13,14-dihydroretinoids” are used interchangeably herein and includes but not limited to dihydroretinol, dihydroretinal, dihydroretinyl acetate that can be detected in a retinoid-mix according to the present invention. The skilled person known how to measure this.

Particularly, the present invention is related to a process for reducing the percentage of by-products including e.g. retinal and dihydroretinoids present in a retinoid-mix as defined herein, particularly wherein a retinoid-producing host cell, such as a retinal/retinol-producing host cell is modified leading to (over) expression of enzymes having RDH22 activity, particularly YlRDH22 or WaRDH22 and homologs thereof as defined herein, wherein the reduction of retinal is in the range of 20 to more than 92%, particularly about 25, 30, 40, 50, 70, 75, 80, 85, 90, 92, 95% or more and/or the reduction of dihydroretinoids is in the range of 20 to more than 97%, particularly about 25, 30, 40, 50, 70, 75, 80, 85, 90, 92, 95, 97% or more as compared to a process using a non-modified host cell, i.e. a host cell that has not been transformed with such heterologous RDH22 polynucleotides as defined herein.

As used herein, a “modified host cell” refers to a host cell or a process wherein the host cell is (over) expressing a heterologous RDH22 homolog as defined herein, including but not limited to host cells modified such that the activity of an endogenous RDH22 homolog as defined herein is increased as compared to the wild-type or non-modified host cell, such as e.g. via expression of 2, 3, 4, or more gene copies of an endogenous gene, i.e. expression of more copies of the respective endogenous gene as occurring in the respective non-modified wild-type host cell. A modified host cell is also known as “recombinant host cell”.

As used herein, a “non-modified host cell” or “wild-type host cell”-which is used interchangeably herein-refers to the respective host cell that is not transformed with a heterologous RDH22 homolog as defined herein and/or that is not over-expressing an endogenous RDH22 homolog, such as e.g. an enzyme having YlRDH22 or WaRDH22 activity as defined herein.

Modifications in order to generate a recombinant host cell having increased activity of an RDH22 homolog as defined herein, may include the use of strong promoters, suitable transcriptional- and/or translational enhancers, or the introduction of one or more gene copies into the respective wild-type host cell, leading to increased accumulation of the respective enzymes and the respective products (e.g. retinol, retinyl acetate) in a given time. The skilled person knows which techniques to use in dependence of the host cell. The increase (or reduction) of gene expression can be measured by various methods, such as e.g. Northern, Southern, or Western blot technology as known in the art.

Expression of the novel RDH22 homologs as defined herein can be achieved in any host system, including (micro) organisms, which is suitable for conversion of retinal into retinol (i.e. retinol-producing host cell) and further suitable for conversion of retinol into retinyl acetate (i.e. retinyl-acetate producing host cell) and furthermore suitable for conversion of carotenoid precursors into beta-carotene (i.e. carotenoid-producing host cell) and furthermore suitable for conversion of beta-carotene into retinal (i.e. retinal-producing host cell) and which allows the expression of the enzymes as disclosed herein and as used for such processes. Examples of suitable host cells to be used for the present invention might be selected from bacteria, algae, fungi including yeast, plant or animal cells, such as e.g. fungal host cells including oleaginous yeast cells, such as e.g. Rhodosporidium, Lipomyces, Saccharomyces or Yarrowia, preferably Yarrowia, more preferably Yarrowia lipolytica or bacterial host cells including but not limited to Escherichia or Pantoea. The skilled person knows which genes are suitable in order to express the necessary enzymes involved in carotenoid—and/or retinoid biosynthesis in a suitable host cell. Genes and methods to generate carotenoid-producing host cells are known in the art, see e.g. WO2006102342. Depending on the carotenoid to be produced, different genes might be involved.

As used herein, the term “fungal host cell” particularly includes yeast cells, such as e.g. retinal/retinol/retinyl-acetate-producing yeast cells, comprising Yarrowia or Saccharomyces. As used herein, the term retinyl-acetate producing host cells” includes host cells, capable of synthesizing retinol and expressing acetyl transferases (ATFs) as defined in e.g. WO2019058001 or WO2020141168 resulting in retinyl acetate with a percentage as defined herein based on total retinoids produced by said host cell. Optionally, such host cell is furthermore capable of producing carotenoids.

Preferably, the host cell expressing an enzyme having activity of YlRDH22 or WaRDH22 as defined herein, including enzymes with at least about 30% identity to YlRDH22 according to SEQ ID NO:1 or WaRDH22 according to SEQ ID NO:4 to be used for fermentative production of a retinoid-mix according to the present invention comprises further modifications enabling the conversion of retinol into retinyl acetate via action/expression of heterologous ATF [EC 2.3.1.84], 20) particularly fungal ATF, comprising a highly conserved partial amino acid sequence of at least 7 amino acid residues selected from [NDEHCS]-H-x(3)-D-[GA] (motifs are in Prosite syntax, as defined in https://prosite.expasy.org/scanprosite/scanprosite_doc.html), wherein “x” denotes an arbitrary amino acid and with the central histidine being part of the enzyme's binding pocket, preferably wherein the 7 amino acid motif is selected from [NDE]-H-x(3)-D-[GA], more preferably selected from [ND]-H-x (3)-D-[GA], most preferably selected from N—H-x (3)-D-[GA] corresponding to position N218 to G224 in the polypeptide according to SEQ ID NO:1 as disclosed in WO2020141168. Examples of such enzymes might be particularly selected from L. mirantina, L. fermentati, S. bayanus, or W. anomalus, such as disclosed in WO2020141168 or WO2019058001, more preferably said ATFs comprising one or more amino acid substitution(s) in a sequence with at least about 20%, such as e.g. 25, 30, 35, 40, 45, 50, 55, 60, 65, 70, 75, 80, 85, 90, 92, 95, 97, 98, 99% or up to 100% identity to LmATF1 (i.e. SEQ ID NO:1 as disclosed in WO2020141168), wherein the one or more amino acid substitution(s) are located at position(s) corresponding to amino acid residue(s) selected from the group consisting of position 68, 69, 72, 73, 171, 174, 176, 178, 291, 292, 294, 301, 307, 308, 311, 312, 320, 322, 334, 362, 405, 407, 409, 480, 483, 484, 490, 492, 520, 521, 522, 524, 525, 526 and combinations thereof and as particularly exemplified in Table 4 of WO2020141168, most preferably comprising one or more amino acid substitution(s) on positions corresponding to amino acid residue(s) 69, 407, 409, 480, 484, and combinations thereof in SEQ ID NO:1 as disclosed in WO2020141168, even more preferably comprising an amino acid substitution at a position corresponding to residue 69 in the polypeptide according to SEQ ID NO: 1 as disclosed in WO2020141168 leading to asparagine, serine or alanine at said residue, such as e.g. via substitution of histidine by asparagine (H69N), serine (H69S) or alanine (H69A), with preference for H69A, optionally being combined with amino acid substitution at a position corresponding to residue 407 in the polypeptide according to SEQ ID NO:1 as disclosed in WO2020141168 leading to isoleucine at said residue, such as e.g. via substitution of valine by isoleucine (V4071), optionally being combined with an amino acid substitution at a position corresponding to residue 409 in the polypeptide according to SEQ ID NO: 1 as disclosed in WO2020141168 leading to alanine at said residue, such as e.g. via substitution of glycine by alanine (G409A), optionally being combined with amino acid substitution at a position corresponding to residue 480 in the polypeptide according to SEQ ID NO:1 as disclosed in WO2020141168 leading to glutamic acid, lysine, methionine, phenylalanine or glutamine at said residue, such as e.g. via substitution of serine by glutamic acid (S480E), lysine (S480L), methionine (S480M), phenylalanine (S480F) or glutamine (S480Q), optionally being combined with amino acid substitution at a position corresponding to residue 484 in the polypeptide according to SEQ ID NO:1 as disclosed in WO2020141168 leading to leucine at said residue, such as e.g. via substitution of isoleucine by leucine (1484L). Said modified enzyme might be originated from yeast, such as e.g. L. mirantina, L. fermentati, W. anomalus or S. bayanus, preferably from L. mirantina. In a most preferred embodiment, the ATF to be used for the process according to the present invention is a modified ATF1 comprising amino acid substitutions S480Q_G409A_V4071_H69A_1484L and is obtainable from Lachancea mirantina, including an enzyme with an identity of at least about 20% to the LmATF1 according to SEQ ID NO:1 as disclosed in WO2020141168.

Suitable culture conditions of a fermentation process according to the present invention include cultivation of the host cell in an aqueous medium in the presence of suitable carbon sources as defined herein, optionally supplemented with appropriate nutrients under aerobic or anaerobic conditions and as known by the skilled person to enable production of a retinoid-mix as defined herein. The fermentation may be conducted in batch, fed-batch, semi-continuous or continuous mode. Particularly, fermentations are run in fed-batch stirred tank reactors. Fermentations can be run for 5 to 14 days, such as e.g. for around 118 h. Fermentation products including but not limited to retinol, retinyl acetate may be harvested from the cultivation at a suitable moment, e.g. when the tank fills due to addition of the feed. The retinoid-mix comprising retinol and acetylated retinoids might be further purified and/or further processed/formulated, to be used in the food, feed, pharma or cosmetic industry. Cultivation and isolation of host cells selected from Yarrowia or Saccharomyces to be used for production of carotenoids and/or retinoids is described in e.g. WO2008042338.

Carbon sources to be used for the present invention might be selected from linear alkanes, free fatty acids, including triglycerides, particularly vegetable oil, such as e.g. selected from the group consisting of oil originated from corn, soy, olive, sunflower, canola, cottonseed, rapeseed, sesame, safflower, grapeseed or mixtures thereof, including the respective free fatty acids, such as e.g. oleic acid, palmitic acid or linoleic acid. Suitable carbon sources might furthermore be selected from ethanol, glycerol or glucose and mixtures of one or more of the above-listed carbon sources.

Particularly, the present invention is directed to a process for the production of retinoids, in particular a retinoid-mix as defined herein, in a two-phase culture system including an in vitro extraction system, i.e. cultivation in the presence of a lipophilic solvent as defined herein, wherein a retinal-/retinol-/retinyl acetate-producing host cell, preferably oleaginous yeast cell such as e.g. Yarrowia, is cultivated under suitable culture conditions such that the retinoid-mix, including retinyl acetate, is accumulated in the lipophilic solvent and optionally extracted/isolated and/or purified from said lipophilic solvent.

Preferred lipophilic solvents to be used for this aspect of the present invention might be selected from isoparaffins including mixtures of alkanes, cycloparaffin, isoalkanes, cycloalkanes, or dodecanes. The solvents might be natural or synthetic ones. Examples of commercially available useful solvents might be selected from Total, e.g. Isane® solvents, Shell, e.g. ShellSolTD or ShellSolT, Exxon Mobile, e.g. Isopar™ fluids, particularly such as e.g. Isopar M, Isopar N, Isopar H, Isopar K, Isopar L, or mixtures with iso-dodecane isomers, as e.g. commercially available under the tradename AC365770010 (Acros Organics).

Preferably, the second phase solvent is selected from isoparaffins, such as e.g. Isopar M, Isopar N, Isopar H, Isopar K, Isopar L, more preferably selected from Isopar L, Isopar M or Isopar N, solvents with equivalent or identical properties but from other suppliers, wherein said solvents are preferably not being consumed or evaporated during the fermentation process, and/or wherein the color of the produced retinyl acetate is transparent.

In one particular embodiment, the host cell to be used for production of a retinoid-mix according to the present invention comprises further modifications, such as modification in endogenous enzyme activities leading to conversion of retinol into fatty acid retinyl esters (FAREs). Particularly, such modifications include reduction or deletion of endogenous lipase activities as dependent on the corresponding host cell, particularly reduction or deletion of activity of one or more endogenous gene(s) encoding enzymes with activity equivalent to Yarrowia LIP2 and/or LIP3 and/or LIP4 and/or LIP8, being reduced or abolished, such as polypeptides with at least about 50%, such as 60, 70, 80, 90, 95, 98, or 100% identity to SEQ ID NO:7, 9, 11, 13, or combinations thereof as disclosed in WO2021136689, wherein SEQ ID NO:7 of WO2021136689 corresponds to LIP2 obtainable from Yarrowia lipolytica, SEQ ID NO:9 of WO2021136689 corresponds to LIP3 obtainable from Yarrowia lipolytica, SEQ ID NO:11 of WO2021136689 corresponds to LIP4 obtainable from Yarrowia lipolytica, SEQ ID NO:13 of WO2021136689 corresponds to LIP8 obtainable from Yarrowia lipolytica. Preferably, the process as defined herein comprising an RDH22 homolog as described herein is modified in the activity of a lipase corresponding to activity of Yarrowia LIP8, such as particularly with reduced or abolished activity, more particularly abolished LIP8 activity, including reduced or abolished activity of a gene encoding a lipase with activity corresponding to LIP8 activity from Yarrowia lipolytica, more preferably wherein a polypeptide with at least about 50% identity to SEQ ID NO:13 of WO2021136689 is abolished. A process for reduction of FARE using a host cell with reduced lipase activity is known from e.g. WO2021136689.

The term “lipase” is used interchangeably herein with the term “enzyme having lipase activity”. It refers to enzymes involved in pre-digestion of triglyceride oils such as e.g. vegetable oil into glycerol and fatty acids that are normally expressed in oleaginous host cells. Suitable enzymes to be modified in a host cell as defined herein might be selected from endogenous enzymes belonging to EC class 3.1.1.-, including, but not limited to one or more enzyme(s) with activities corresponding to Yarrowia LIP2, LIP3, LIP4, or LIP8 activities.

As used herein, an enzyme having “reduced or abolished” activity means a decrease in its specific activity, i.e. reduced/abolished ability to catalyze formation of a product from a given substrate. A reduction by 100% is referred herein as abolishment of enzyme activity, achievable e.g. via deletion, insertions, frameshift mutations, missense mutations or premature stop-codons in the endogenous gene encoding said enzyme or blocking of the expression and/or activity of said endogenous gene(s) with known methods.

The terms “sequence identity”, “% identity” are used interchangeable herein. For the purpose of this invention, it is defined here that in order to determine the percentage of sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. The percent 20) sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids, they are identical or have 100% identity.

The enzymes as described herein to be expressed in a suitable host cell to be used in the present invention also encompass enzymes carrying (further) amino acid substitution(s) which do not alter enzyme activity, i.e. which show the same properties with respect to the enzymes defined herein. Such mutations are also called “silent mutations”. Examples of silent mutations included in the present invention are host-optimized sequences, including but not limited to SEQ ID NO: 3 or 6.

Thus, in a preferred embodiment the present invention is related to production of a retinoid-mix as defined herein, comprising a percentage of dihydroretinoids of less than about 10% based on total retinoids, comprising a percentage of at least about 80% retinyl acetate based on total retinoids, comprising a percentage of less than about 8% retinal based on total retinoids, wherein the process is conducted in Yarrowia lipolytica, particularly wherein endogenous lipase activities are reduced or completely abolished, such as e.g. activity of LIP8, furthermore comprising reduction or complete abolishment of lipase activities including endogenous LIP2, and/or LIP3, and/or LIP4, said host cell furthermore expressing a heterologous ATF1 homolog, preferably fungal ATF1 with activity of LmATF1, LfATF1, LffATF1, SbATF1 as e.g. disclosed in WO2019058001 or a modified ATF1 comprising amino acid substitutions S480Q_G409A_V4071_H69A_1484L such as obtainable from Lachancea mirantina, including an enzyme with an identity of at least about 20% to the LmATF1 according to SEQ ID NO:1 as disclosed in WO2020141168, and wherein said host cell comprises and expresses an RDH22 homolog as defined herein, such as e.g. RDH22 with at least about 30% identity to SEQ ID NO:1 or 4.

With regards to the present invention, it is understood that organisms, such as e.g. microorganisms, fungi, algae or plants also include synonyms or basonyms of such species having the same physiological properties, as defined by the International Code of Nomenclature of Prokaryotes or the International Code of Nomenclature for algae, fungi, and plants (Melbourne Code). Thus, for example, strain Lachancea mirantina is a synonym of strain Zygosaccharomyces sp. IFO 11066, originated from Japan.

As used herein, the term “specific activity” or “activity” with regards to enzymes means its catalytic activity, i.e. its ability to catalyze formation of a product from a given substrate. The specific activity defines the amount of substrate consumed and/or product produced in a given time period and per defined amount of protein at a defined temperature. Typically, specific activity is expressed in μmol substrate consumed or product formed per min per mg of protein. Typically, μmol/min is abbreviated by U (=unit). Therefore, the unit definitions for specific activity of μmol/min/(mg of protein) or U/(mg of protein) are used interchangeably throughout this document.

An enzyme is active, if it performs its catalytic activity in vivo, i.e. within the host cell as defined herein or within a suitable (cell-free) system in the presence of a suitable substrate. The skilled person knows how to measure enzyme activity. Analytical methods to evaluate the capability/enzymatic activity of a suitable enzyme involved in retinoid production are known in the art, such as e.g. described in Example 4 of WO2014096992. In brief, titers of products such as dihydroretinoids, retinyl acetate, retinol, retinal, beta-carotene, and the like can be measured by HPLC.

As used herein, an enzyme is “expressed and active in vivo” if mRNA encoding for the protein can be detected by Northern blotting and/or protein is detected by mass spectrometry. With regards to the lipase activity as defined herein it means ability of the host cell to utilize triglycerides according to the definition herein. With regards to ATFs as defined herein it means ability of a host cell for acetylation of retinol into retinyl acetate. With regards to RDH activity as defined herein it means ability of a host cell for conversion of retinal into retinol.

Retinoids as used herein include beta-carotene cleavage products also known as apocarotenoids, including but not limited to retinal, retinolic acid, retinol, retinoic methoxide, retinyl acetate, retinyl esters, 4-keto-retinoids, 3 hydroxy-retinoids, dihydroretinoids or combinations thereof. A mixture comprising one or more of said beta-carotene cleavage products, particularly retinal, retinol, retinyl acetate, and/or dihydroretinoid is defined herein as “retinoid-mix” or “total retinoids”, wherein said retinoids are accumulated during the fermentation process with typical measurement at the end of fermentation process. Biosynthesis of retinoids is described in e.g. WO2008042338.

A host cell capable of production of retinoids/retinoid-mix as defined herein in e.g. a fermentation process is known as “retinoid-producing host cell” and includes a host cell producing e.g. retinal, retinol and/or retinyl acetate. As used herein, a “retinal-producing host cell” is a host cell wherein the respective polypeptides are expressed and active in vivo, leading to production of retinal, e.g. via enzymatic conversion of beta-carotene into retinal. As used herein, a “retinol-producing host cell” is a host cell, wherein the respective polypeptides are expressed and active in vivo, leading to production of retinol, e.g. via enzymatic conversion of retinal into retinol. A “retinyl acetate-producing host cell” is the respective host cell capable of acetylation of retinol into retinyl acetate via expression of the respective acetylating enzymes, e.g. ATFs, preferably ATF1 enzymes, as described herein.

“Retinal” as used herein is known under IUPAC name (2E,4E,6E,8E)-3,7-Dimethyl-9-(2,6,6-trimethylcyclohexen-1-yl) nona-2,4,6,8-tetraenal. It is herein interchangeably referred to as retinaldehyde or vitamin A aldehyde and includes both cis- and trans-isomers, such as e.g. 11-cis retinal, 13-cis retinal, trans-retinal and all-trans retinal.

The term “carotenoids” as used herein is well known in the art. It includes long, 40 carbon conjugated isoprenoid polyenes that are formed in nature by the ligation of two 20 carbon geranylgeranyl pyrophosphate molecules. These include but are not limited to phytoene, lycopene, and carotene, such as e.g. beta-carotene, which can be oxidized on the 4-keto position or 3-hydroxy position to yield canthaxanthin, zeaxanthin, or astaxanthin. Biosynthesis of carotenoids is described in e.g. WO2006102342.

“Vitamin A” as used herein may be any chemical form of vitamin A found in aqueous solutions, in solids and formulations, and includes retinol, retinyl acetate and retinyl esters. It also includes retinoic acid, such as for instance undissociated, in its free acid form or dissociated as an anion. A preferred form of vitamin A is retinyl acetate, wherein the terms “retinyl acetate”, “retinol acetate” and “vitamin A acetate” might be used interchangeably (see https://www.cancer.gov/publications/dictionaries/cancer-drug/def/retinyl-acetate?redirect=true).

The terms “triglycerides” and “triglyceride oils” are used interchangeably herein. “FAREs” or “retinyl fatty esters” as used interchangeably herein includes long chain retinyl esters. These long chain retinyl esters define hydrocarbon esters that consists of at least about 8, such as e.g. 9, 10, 12, 13, 15 or 20 carbon atoms and up to about 26, such as e.g. 25, 22, 21 or less carbon atoms, with preferably up to about 6 unsaturated bonds, such as e.g. 0, 1, 2, 4, 5, 6 unsaturated bonds. Long chain retinyl esters include but are not limited to linoleic acid, oleic acid, or palmitic acid.

Particularly, the present invention features the following embodiments 1 to 14:
Embodiment 1: Process for production of a retinoid mix comprising dihydroretinoids, retinal, retinol, and retinyl acetate, wherein the percentage of dihydroretinoids in said mix is less than about 10% or based on total retinoids.
Embodiment 2: Process of embodiment 1, wherein the percentage of retinyl acetate in said mix is at least about 80% based on total retinoids.
Embodiment 3: Process of embodiment 1 or 2, wherein the percentage of retinyl acetate in said mix is at least about 80% based on total retinoids.
Embodiment 4: Process of embodiment 1, 2, or 3, wherein said mix comprises trans and cis-isomers and wherein the percentage of trans-isomers is of at least 80% based on total cis and trans-isomers in said mix.
Embodiment 5: Process of embodiment 4, wherein the percentage of trans-retinyl acetate in said mix is at least about 80%.
Embodiment 6: Process of embodiment 1, 2, 3, 4, or 5, wherein said mix comprising dihydroretinal, dihydroretinol and dihydroretinyl acetate.
Embodiment 7: A retinol-producing host cell expressing a retinol dehydrogenase 22 (RDH22) homolog with at least about 30% identity to a polynucleotide according to SEQ ID NO:1 or 4, wherein the host cell has been transformed with a polynucleotide encoding said RDH22 homolog.
Embodiment 8: Retinol-producing host cell of embodiment 7, wherein the RDH22 homolog is obtained from Yarrowia or Wickerhamomyces.
Embodiment 9: Use of the retinol-producing host cell of embodiment 7 or 8 in a process according to embodiment 1, 2, 3, 4, 5, or 6.
Embodiment 10: Use of a RDH22 homolog with at least about 30% identity to a polynucleotide according to SEQ ID NO:1 or 4 in a process according to embodiment 1, 2, 3, 4, 5, or 6, wherein the RDH22 homolog is expressed in a suitable retinol-producing host cell.
Embodiment 11: Use of RDH22 homolog having activity of YlRDH22 according to SEQ ID NO:1 in a fermentative production of a retinoid-mix with at least about 80% retinyl acetate within said mix using a suitable retinyl acetate producing host cell, wherein the percentage of retinal within said mix and accumulated during said process is reduced to less than 1% based on total retinoids and measured at the end of the production process.
Embodiment 12: Use of RDH22 homolog having activity of WaRDH22 according to SEQ ID NO:4 in a fermentative production of a retinoid-mix with at least about 80% retinyl acetate within said mix using a suitable retinyl acetate producing host cell, wherein the percentage of retinal within said mix and accumulated during said process is reduced to about 2% or less based on total retinoids and measured at the end of the production process.
Embodiment 13: Process according to embodiment 1, 2, 3, 4, 5, or 6, wherein a host cell according to embodiment 7 or 8 is cultivated in the presence of a lipophilic solvent.
Embodiment 14: Process according to embodiment 13, wherein the retinoid mix is accumulated in the lipophilic solvent and optionally extracted from the lipophilic solvent.

The following examples are illustrative only and are not intended to limit the scope of the invention in any way. The contents of all references, patent applications, patents, and published patent applications, cited throughout this application are hereby incorporated by reference, particularly WO2019057999, WO2021009194, WO2020141168, WO2019058000, WO2019058001, WO2006102342, WO2008042338, WO2021136689, WO2014096992, WO2016172282.

### EXAMPLES

### Example 1: General Methods and Plasmids

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998).

Shake plate assay. Typically, 200 μl of 0.075% Yeast extract, 0.25% peptone (0.25X YP) is inoculated with 10 μl of freshly grown Yarrowia and overlaid with 200 μl of Isopar M with 2% oleic acid as a carbon source. Clonal isolates of transformants were grown in 24 well plates (Multitron, 30° C., 800 RPM) in YPD media with Isopar M overlay indicated earlier for 4 days. The overlay fraction was removed from the shake plate wells and analyzed by HPLC on a normal phase column, with a photo-diode array detector.

DNA transformation. Strains were transformed by overnight growth on YPD plate media; 50 μl of cells were scraped from a plate and transformed by incubation in 500 μl with 1 μg transforming DNA, typically linear DNA for integrative transformation, 40% PEG 3550MW, 100 mM lithium acetate, 50 mM Dithiothreitol, 5 mM Tris-Cl pH 8.0, 0.5 mM EDTA for 60 minutes at 40° C. and plated directly to selective media or in the case of dominant antibiotic marker selection the cells were out grown on YPD liquid media for 4 hours at 30° C. before plating on the selective media. Nourseothricin (Nat) selection was performed on YPD media containing 100 μg/mL nourseothricin and hygromycin (Hyg) selection was performed on YPD containing 100 μg/mL hygromycin. URA3 marker recycling was performed using 5-fluoroorotic acid (FOA). Episomal hygromycin resistance marker (Hyg) plasmids were cured by passage on non-selective media, with identification of Hyg-sensitive colonies by replica plating colonies from non-selective media to hygromycin containing media (100 μg/mL).

DNA molecular biology. Plasmid MB9523 containing expression systems for DrBCO, LmATF-S480Q_G409A_V4071_H69A_1484L, and FfRDH (SEQ ID NO:8) was synthesized at Genscript (Piscataway, NJ, USA). Plasmid MB9523 contains the ‘URA3’ for marker selection in Yarrowia lipolytica transformations. Genes were synthesized with Nhel and Mlul ends in pUC57 vector. Typically, the genes were subcloned to the MB5082 ‘URA3’, MB6157 HygR, and MB8327 NatR vectors for marker selection in Yarrowia lipolytica transformations, as in WO2016172282. For clean gene insertion by random nonhomologous end joining of the gene and marker Hindlll/Xbal (for MB5082-based vectors (all marked with URA3 in Table 1, except MB9523), Pvull (for MB6157—and MB8327-based vectors), or Sfil (for MB9523), respectively purified by gel electrophoresis and Qiagen gel purification column. Clones were verified by sequencing. Typically, genes are synthesized by a synthetic biology at GenScript (Piscataway, NJ). Plasmid MB8388-LIP8 (SEQ ID NO: 9), containing a Cas9, and guide RNA expression systems to target LIP8, was synthesized at Genscript (Piscataway, NJ, USA).

Plasmid list. Plasmid, strains, nucleotide and amino acid sequences that were used are listed in Table 1, 2, 6 and the sequence listing. In general, all non-modified sequences referred to herein are the same as the accession sequence in the database for reference strain CLIB122 (Dujon B, et al, Nature. 2004 Jul. 1;430 (6995): 35-44).

TABLE 1
list of plasmids used for construction of the strains for overexpression
or deletion of the respective genes indicated as “insert”. “LmATF1-mut” refers to
Lachancea mirantina (LmATF1; SEQ ID NO: 13 in WO2019058001) carrying aa
substitutions S480Q_G409A_V4071_H69A_1484L. “DrBCO” refers to BCO originated
from Danio rerio (see SEQ ID NO: 16 in WO2020141168); “FfRDH″ refers to RDH
originated from Fusarium (see SEQ ID NO: 22 in WO2020141168). For more
explanation, see text.

| | | | SEQ |
|---|---|---|---|
| Plasmid | Insert | Marker | ID NO: |
| MB7452 | Cas9 | Nat | 7 |
| MB9523 | DrBCO; LmATF1-mut; FfRDH | URA3 | 8 |
| MB8388-LIP8 | Cas9; LIP8 targeting guide RNAs | Hyg | 9 |
| MB8203 | Homo sapiens RDH12 (HsRDH12) | URA3 | 10 |
| MB9894 | YALIOF29821p (YIRDH22) | URA3 | 11 |
| MB9997 | Wickerhamomyces anomalus RDH22 (WaRDH22) | URA3 | 12 |

TABLE 2
list of Yarrowia lipolytica strains used. Construction of ML17544 is
described in Table 2 of WO2020141168. For more details, see text.

| Strain | Description |
|---|---|
| ML18812 | ML17544 transformed with MB9523 |
| ML18812-lip8 | ML18812 with MB7542 and MB8388-LIP8, lip8 deletion |
| ML18812-lip8 + MB9894 | ML18812-lip8 cured on FOA with MB9894 |
| ML18812-lip8 + MB8203 + MB9894 | ML18812-lip8 + MB8203 cured on FOA, transformed |
| | with MB9894 |

Normal phase retinol method. A Waters 1525 binary pump attached to a Waters 717 auto sampler were used to inject samples. A Phenomenex Luna 3u Silica (2), 150×4.6 mm with a security silica guard column kit was used to resolve retinoids. The mobile phase consists of either, 1000 mL hexane, 30 mL isopropanol, and 0.1 mL acetic acid for astaxanthin related compounds, or 1000 mL hexane, 60 mL isopropanol, and 0.1 mL acetic acid for zeaxanthin related compounds. The flow rate for each was 0.6 mL per minute. Column temperature was ambient. The injection volume was 20 μL. The detector was a photodiode array detector collecting from 210 to 600 nm. Analytes were detected according to Table 3.

TABLE 3
list of analytes using normal phase retinol method. “RT” means
retention time. For more details, see text.

| | | Lambda max |
|---|---|---|
| Intermediates | RT [min] | [nm] |
| 11-cis-dihydro-retinol | 7.1 | 293 |
| 11-cis-retinal | 4 | 364 |
| 11-cis-retinol | 8.6 | 318 |
| 13-cis-retinal | 4.1 | 364 |
| dihydro-retinol | 9.2 | 292 |
| retinyl-acetate | 3.5 | 326 |
| retinyl-ester | 3 | 325 |
| trans-retinal | 4.7 | 376 |
| trans-retinol | 10.5 | 325 |

Sample preparation. Samples were prepared by various methods depending on the conditions. For whole broth or washed broth samples the broth was placed in a Precellys® tube weighed and mobile phase was added, the samples were processed in a Precellys® homogenizer (Bertin Corp, Rockville, MD, USA) on the highest setting 3X according to the manufactures directions. In the washed broth the samples were spun in a 1.7 ml tube in a microfuge at 10000 rpm for 1 minute, the broth decanted, 1 ml water added mixed pelleted and decanted and brought up to the original volume the mixture was pelleted again and brought up in appropriate amount of mobile phase and processed by Precellys® bead beating. For analysis of mineral oil fraction, the sample was spun at 4000 RPM for 10 minutes and the oil was decanted off the top by positive displacement pipet (Eppendorf, Hauppauge, NY, USA) and diluted into mobile phase mixed by vortexing and measured for retinoid concentration by HPLC analysis.

Fermentation conditions. Fermentations were identical to the previously described conditions using Isopar M overlay and stirred tank that was ethanol fed in a bench top reactor with 0.5L to 5L total volume (see WO2016172282). Generally, the same results were observed with a fed batch stirred tank reactor with an increased productivity demonstrating the utility of the system for the production of retinoids.

### Example 2: Production of Retinoids in Yarrowia lipolytica

Strains were grown in the shake plate assay described in Example 1. Addition of plasmids MB9894 (SEQ ID NO:11) or MB9997 (SEQ ID NO:12) to strain ML18812-lip8 improved total retinoid production, when compared to the parental strain which did not contain YlRDH22 or WaRDH22 expression cassettes. Addition of the HsRDH12 expression plasmid MB8203 (SEQ ID NO:10) into strain ML18812-lip8+MB9894 (which results in strain ML18812-lip8+MB8203+MB9894) resulted in a further increased retinoid output. In addition, introduction of MB9894 or MB9997 to ML18812-lip (to make ML18812-lip8+MB9894) reduced the retinal content. The strain ML18812-lip8+MB9894+MB8203 displayed a further reduction of the relative retinal content of the retinoids, and improved the purity of the retinyl acetate, when compared to the parental strain which did not have them. The results are shown in Table 4.

TABLE 4
retinoid production in strain ML18812-lip8 (see Table 2)
as control compared to said strain carrying plasmids
as indicated. “% total retinoids” is the
percentage of retinoids produced compared
to total retinoids in the control
without addition of plasmids, wherein
the total retinoids obtained with the
control is set to 100%. “% retinal” means
percentage of retinal in the retinoid mix based
on total retinoids. For more explanation, see text.

| | % total | % |
|---|---|---|
| Strain | retinoids | retinal |
| ML18812-lip8 | 100 | 8.2 |
| ML18812-lip8 + MB9894 | 114.6 | 0.95 |
| ML18812-lip8 + MB8203 + MB9894 | 136 | 0.6 |
| ML18812-lip8 + MB9997 | 110 | 2.0 |

### Example 3: RDH Expression Diminishes Dihydroretinoid Production

Strains were fermented as described in Example 1, and broth samples were quantified for dihydroretinoid compounds, the results are shown in Table 5. Addition of either HsRDH12 (MB8203), WaRDH22 (MB9997), or YlRDH22 to ML18812-lip8 reduces both retinal and dihydroretinoid fraction, while adding both HsRDH12 and YlRDH22 (MB9894) reduces it further still. Also, addition of either HsRDH12, WaRDH22, YlRDH22, or both HsRDH12 and YlRDH22 increases retinyl acetate purity.

TABLE 5
retinoid production in strain ML18812-lip8
(see Table 2) as control compared to said strain carrying
plasmids as indicated, including the purity with
regards to retinyl acetate, retinal and
dihydroretinoids. The percentages given
are based on total retinoids in the mixture.
For more explanation, see text.

| | % retinyl | % dihydro- |
|---|---|---|
| Strain | acetate | retinoids |
| ML18812-lip8 | 73 | 10.2 |
| ML18812-lip8 + MB9894 | 83 | 0.3 |
| ML18812-lip8 + MB8203 + MB9894 | 92 | 0.6 |
| ML18812-lip8 + MB9997 | 91 | 1.98 |

TABLE 6
list of sequences including protein and polynucleotide sequences.
Nucleotide sequences with a ″*″ refer to codon-optimized sequences. ″aa″ refers
to amino acids sequence, ″nt″ refers to nucleotide sequence. For more details,
see text.

| | | SEQ |
|---|---|---|
| | | ID |
| Name | Sequence 5′ to 3′ | NO: |
| YlRDH22 | MTRSKFTEYKQVLRNSCVGKPQYTEKDYPDLDGKNFLVTGATGGVGLEATKLL | 1 |
| (aa) | LEKKSHVIMVGRSKTKSQSTLDELOKTYSHGTFDFVEADLSDLTTVERAGEYI | |
| | RSKYTTLDGAILNAGVMAPPYSLTPQGHESQWGINVVAHFLLSKYISPALISA | |
| | AQTAPKDTVRLVWVSSSVVAMSPYEGGIKFDDINHSKVKNPSPWTLYSQSKIG | |
| | DAYLAYLWSKHHPDSGVLSVSLDPGNLASDLSRHTSWLSSIKNYVLYPPKYGA | |
| | YTELSALLNPSVKNNEHLIPWGIEGHLRQDVDDGRRGKDGEELWQGLNKDVEG | |
| | FFKEE | |
| YlRDH22 | ATGACTCGATCCAAGTTCACCGAATACAAACAAGTGCTGCGAAACAGCTGCGT | 2 |
| (nt) | GGGCAAGCCCCAATACACCGAGAAGGATTACCCTGATCTCGACGGAAAGAACT | |
| | TTCTTGTCACTGGAGCCACTGGAGGAGTTGGTCTGGAGGCCACCAAGCTGCTG | |
| | CTAGAGAAGAAGAGCCATGTAATCATGGTTGGCCGATCCAAAACAAAGTCTCA | |
| | GTCTACCCTTGACGAGCTCCAGAAGACATACTCCCACGGTACCTTTGATTTCG | |
| | TCGAGGCTGATCTGTCTGACCTGACCACTGTGGAGCGAGCCGGAGAGTACATT | |
| | CGAAGTAAGTACACCACTTTGGACGGTGCCATTCTCAACGCTGGAGTCATGGC | |
| | TCCCCCCTATAGCCTCACTCCCCAGGGCCACGAGTCTCAATGGGGTATCAATG | |
| | TGGTTGCCCATTTTCTGCTGTCCAAGTATATCTCTCCTGCTCTGATCTCGGCC | |
| | GCCCAGACAGCCCCCAAGGACACCGTGCGACTTGTCTGGGTGTCTTCTTCTGT | |
| | CGTTGCCATGAGTCCTTATGAAGGAGGTATCAAGTTTGACGATATCAACCACT | |
| | CTAAGGTCAAGAACCCTTCTCCCTGGACCCTTTACTCCCAGTCCAAGATTGGA | |
| | GATGCTTACCTGGCTTACCTGTGGTCGAAACACCATCCCGATTCTGGCGTCCT | |
| | GTCCGTCTCTCTGGACCCCGGTAACCTGGCCTCTGATCTGTCCAGACATACTT | |
| | CTTGGCTCTCCAGCATTAAGAACTACGTGCTGTATCCTCCCAAGTATGGCGCC | |
| | TACACCGAGCTGTCTGCTCTGCTGAACCCCTCCGTCAAGAATAACGAGCATCT | |
| | CATTCCCTGGGGTATTGAGGGACACCTTAGACAGGATGTCGACGATGGACGAC | |
| | GTGGAAAGGACGGAGAAGAGCTCTGGCAGGGTCTCAACAAGGATGTCGAGGGC | |
| | TTCTTTAAGGAGGAGTAA | |
| YlRDH22 | ATGACCCGATCCAAGTTCACCGAGTACAAGCAGGTTCTCCGAAACTCT | 3 |
| (nt*) | TGTGTTGGCAAGCCCCAGTACACTGAGAAGGATTACCCCGATCTCGAC | |
| | GGAAAGAACTTTCTCGTCACTGGTGCTACTGGTGGAGTTGGTCTGGAG | |
| | GCCACTAAGCTGCTCCTTGAGAAGAAGTCTCACGTTATTATGGTTGGC | |
| | CGATCTAAGACTAAGTCTCAGTCTACCCTTGACGAGCTTCAGAAGACT | |
| | TACTCTCACGGTACTTTCGATTTCGTTGAGGCTGATCTCTCTGACCTT | |
| | ACCACTGTTGAGCGAGCCGGAGAGTACATTCGATCTAAGTACACTACC | |
| | CTTGACGGTGCCATTCTTAACGCTGGAGTCATGGCTCCCCCCTACTCT | |
| | CTCACTCCCCAGGGTCACGAGTCTCAGTGGGGTATTAACGTTGTTGCT | |
| | CACTTTCTCCTGTCTAAGTACATCTCTCCTGCTCTGATTTCTGCTGCT | |
| | CAGACTGCCCCCAAGGACACCGTTCGACTTGTTTGGGTGTCTTCTTCT | |
| | GTCGTTGCCATGTCTCCTTACGAGGGAGGTATTAAGTTTGACGATATT | |
| | AACCACTCTAAGGTCAAGAACCCTTCTCCTTGGACCCTTTACTCTCAG | |
| | TCTAAGATTGGTGATGCTTACCTGGCTTACCTTTGGTCTAAGCACCAC | |
| | CCCGATTCTGGAGTCCTTTCTGTCTCTCTGGACCCCGGTAACCTGGCT | |
| | TCTGATCTCTCTCGACACACTTCTTGGCTTTCTTCTATTAAGAACTAC | |
| | GTTCTTTACCCTCCCAAGTACGGTGCCTACACTGAGCTGTCTGCTCTT | |
| | CTCAACCCTTCCGTTAAGAACAACGAGCACCTTATTCCCTGGGGTATT | |
| | GAGGGACACCTTCGACAGGATGTCGACGATGGTCGACGAGGAAAGGAC | |
| | GGAGAGGAGCTCTGGCAGGGTCTTAACAAGGATGTCGAGGGTTTCTTT | |
| | AAGGAGGAGTAA | |
| WaRDH22 | MVNVAYEWLRDCWPPKPEFLPKDYPDLTGKTILITGANTGIGFESAKA | 4 |
| (aa) | LLKKNATVVFANRSEEKTKIAIAKIQAELGGDSSQRSIFVKTDLSDLS | |
| | SIKGTAEELKSKGITKLHYTILNAGVMQPPKGSKTKQGFELQIGTNVL | |
| | GHQLLQKFLTPLVLNAVSSDFTPRVVWLASAAHLSSPPNGGIDWDSER | |
| | NADWAGTVSAYGQSKTGNIYQAYIYAQQHKDVISVSAHPGYLASDLTR | |
| | AYKGYLQYLMKLLMSPPVYGSYTELFAALSPKVTLKESGRYIGPWGOF | |
| | RELRDDVQEGLTDGTAQKLWDWAEAEIRPYT | |
| WaRDH22 | ATGGTTAACGTTGCTTATGAATGGCTAAGAGACTGCTGGCCTCCTAAACCTGA | 5 |
| (nt) | ATTCTTACCAAAAGATTATCCAGATTTAACAGGAAAGACAATCTTAATCACTG | |
| | GTGCCAATACGGGTATTGGGTTTGAAAGTGCTAAAGCCTTGTTGAAGAAAAAC | |
| | GCAACAGTTGTATTTGCAAATAGAAGCGAAGAGAAGACAAAAATCGCTATTGC | |
| | CAAAATTCAAGCTGAATTAGGTGGTGATTCATCTCAAAGATCAATTTTCGTCA | |
| | AGACTGATTTAAGTGATTTATCATCGATCAAGGGCACAGCGGAAGAATTGAAA | |
| | TCAAAAGGTATCACAAAATTACATTACACCATATTGAATGCTGGTGTTATGCA | |
| | ACCTCCAAAAGGTTCCAAGACAAAACAAGGATTTGAATTACAAATTGGTACAA | |
| | ATGTTTTAGGTCATCAATTATTACAAAAATTTCTAACACCATTAGTTTTGAAT | |
| | GCAGTTTCATCAGATTTCACACCAAGAGTTGTTTGGTTAGCCTCAGCTGCACA | |
| | TCTTTCATCCCCACCAAATGGTGGTATTGATTGGGATTCCTTCCGTAATGCTG | |
| | ATTGGGCTGGAACTGTCAGTGCTTATGGTCAATCCAAGACTGGTAATATTTAT | |
| | CAAGCTTATATCTATGCTCAGCAACATAAAGATGTCATCAGTGTCTCTGCTCA | |
| | TCCAGGGTATTTGGCTTCTGATTTAACAAGAGCTTATAAAGGTTATCTTCAAT | |
| | ATTTGATGAAACTCTTGATGTCTCCTCCAGTTTATGGTTCTTATACTGAATTA | |
| | TTCGCTGCTCTCAGTCCAAAAGTTACATTGAAGGAAAGTGGTAGATATATTGG | |
| | TCCTTGGGGTCAATTCAGAGAATTGAGAGATGATGTTCAAGAAGGCTTAACTG | |
| | ATGGAACTGCTCAAAAATTATGGGATTGGGCTGAAGCTGAAATTAGACCATAC | |
| | ACTTAG | |
| WaRDH22 | ATGGTTAACGTTGCTTACGAGTGGCTTCGAGACTGCTGGCCCCCTAAGCCTGA | 6 |
| (nt*) | GTTCCTCCCTAAGGATTACCCTGACCTCACCGGAAAGACCATCCTTATCACTG | |
| | GTGCCAACACTGGTATTGGTTTTGAGTCCGCTAAGGCTCTCCTCAAGAAGAAC | |
| | GCTACCGTTGTTTTTGCCAACCGATCCGAGGAGAAGACTAAGATCGCTATTGC | |
| | CAAGATTCAGGCTGAGCTTGGTGGTGATTCTTCTCAGCGATCTATTTTCGTCA | |
| | AGACTGACCTTTCCGATCTCTCTTCCATTAAGGGCACTGCTGAGGAGCTTAAG | |
| | TCTAAGGGTATCACTAAGCTCCACTACACCATTCTTAACGCTGGTGTTATGCA | |
| | GCCTCCCAAGGGCTCCAAGACTAAGCAGGGTTTTGAGCTTCAGATTGGTACTA | |
| | ACGTTCTTGGCCACCAGCTCCTCCAGAAGTTTCTCACTCCCCTTGTTCTCAAC | |
| | GCTGTTTCTTCTGATTTCACCCCTCGAGTTGTTTGGCTCGCCTCTGCTGCTCA | |
| | CCTTTCTTCCCCTCCCAACGGTGGTATTGATTGGGATTCCTTCCGAAACGCTG | |
| | ATTGGGCTGGTACTGTCTCTGCTTACGGTCAGTCTAAGACTGGTAACATTTAC | |
| | CAGGCTTACATCTACGCTCAGCAGCACAAGGATGTCATCTCTGTCTCTGCTCA | |
| | CCCCGGATACCTTGCTTCTGATCTCACTCGAGCTTACAAGGGATACCTTCAGT | |
| | ACCTTATGAAGCTCCTTATGTCTCCTCCCGTTTACGGTTCTTACACTGAGCTT | |
| | TTCGCTGCTCTTTCTCCCAAGGTTACTCTCAAGGAGTCTGGTCGATACATTGG | |
| | TCCTTGGGGTCAGTTCCGAGAGCTTCGAGATGACGTTCAGGAGGGCCTTACCG | |
| | ATGGTACTGCTCAGAAGCTCTGGGATTGGGCTGAGGCTGAGATTCGACCTTAC | |
| | ACTTAA | |
| MB7452 | CGCGTGGATCGCCGGTGCGTTGACGTTGGTGACCTCCAGCCAGAGGTGCCCGG | 7 |
| | CGCCCCGCTCGCCGGCGAACTCCGTCGCGAGCCCCATCAACGCGCGCCCGACC | |
| | CCGTGCCCCCGGTGCTCCGGGGCGACCTCGATGTCCTCGACGGTCAGCCGGCG | |
| | GTTCCACGCCGAGTACGAGATGACCACGAAGCCCGCCAGGTCGCCGTCGTCCC | |
| | CGTACGCGACGAACGTCCGGGAGTCCGGGTCGCCGTCCTCCCCGTCGTCCGAT | |
| | TCGTCGTCCGATTCGTCGTCGGGGAACACCTTGGTCAGGGGCGGGTCCACCGG | |
| | CACCTCCCGCAGGGTGAAGCCGTCCCCGGTGGCGGTGACGCGGAAGACGGTGT | |
| | CGGTGGTGAAGGACCCATCCAGTGCCTCGATGGCCTCGGCGTCCCCCGGGACA | |
| | CTGGTGCGGTACCGGTAAGCCGTGTCGTCAAGAGTGGTCATTTTTGTGTCTAG | |
| | GTGTTTGTGTTTGGACTGCGATCAGTGAAGAAAAGAAGAGGAAAAATTGTGCA | |
| | AGAAATTTTGCTTTCAAGACTTGGCTGATGCAGCAGGGTAACTCTGGGACACA | |
| | GACCTATGTTTGTGGTTAAACTCAATGCACGTGGTACGTGCGTGGAGCGCTTA | |
| | CCCATCCAAGGGTGTGGACATGGAACCGACGGTCCGTGGAGTTGTGTAATGTC | |
| | ATTTTGGCGACTCTTGAAGCAAGGCTATAAAAAAATTGTGTGGCTTGAGTCTT | |
| | ATCGAGCTCGGTCACTACAAGAGTTAATCTTCCTGTCTCAGGCAGACAGGTCA | |
| | GGCAGGGTTACTTTTGGGTGTGCTGTAACTCACTGTATGGCCGTTAGTGCGCA | |
| | TAGACGTTGTACATACTGGACCGAATTGTAGCGTGCTCAATAGGGCCAATAAA | |
| | GCTATTGTAGGGATCCGAATTTTCAGAACCTAATTTATCTGTTACCCGGCCTG | |
| | TGGCTCGCACAGCTTAAAAATGGTCAAACTTTCCCCTTCTTGTCTTTTTTTCC | |
| | TCACATTCATCAGGTTCTTGTCTTGATCTTTCAAGTGAGTATTAATTACCGAC | |
| | CTTGGTTCTTCATTGGGAGAGCATTGGAAGCCGTGGTGCAGCAACCACAAAAC | |
| | GGTTCTTCCCCTTCGATACCTTCTTGCCTGCCTTTCAATACAAGTCGGCTCGA | |
| | TTAGCGGTGGTCGCCCCCGCCAGCGGAGAACATGGAACTAACCCAGAATGAGA | |
| | GCTAAGTGGAGAAAGAAGAGAGTCAGACGACTCAAGCGAAAGCGCCGCAAGGT | |
| | CCGAGCTCGATCCAAATAAGCGGTTTTTAACGGAGATTTAACACTAAATCGAA | |
| | GAACTTTTCCCGTTTCATTTGCGAATGAGCTCGTTAACAAAATCCCCCAGTTT | |
| | TTTTATCCAGCTGTAAGGATTGACATTAGTAATGAATTATTGTTTGGTATATT | |
| | TAAATCTGTAGTTCCTTTCTGTCCGTGTCGGCAACTGTCGTACTCGTGATTTA | |
| | CTTGTATTGACGAATACTTACTGTAGCGCACTCTGCTGCTACTGGTCGTAAGG | |
| | ATGTGCTATTTCGGTGTATGGTGGGTTTTTTGGGGGTCGGAACCGAAGACTGT | |
| | TACACGGGCACGGCTCGTTGTGTACACGCACAGAGCTCTTGCGAGTCATGTTG | |
| | TAGCTAGCTCGTCGTGTTCAGGAACTGTTCGATGGTTCGGAGAGAGTCGCCGC | |
| | CCAGAACATACGCGCACCGATGTCAGCAGACAGCCTTATTACAAGTATATTCA | |
| | AGCAAGTATATCCGTAGGGTGCGGGTGATTTGGATCTAAGGTTCGTACTCAAC | |
| | ACTCACGAGCAGCTTGCCTATGTTACATCCTTTTATCAGACATAACATAATTG | |
| | GAGTTTACTTACACACGGGGTGTACCTGTATGAGCACCACCTACAATTGTAGC | |
| | ACTGGTACTTGTACAAAGAATTTATTCGTACGAATCACAGGGACGGCCGCCCT | |
| | CACCGAACCAGCGAATACCTCAGCGGTCCCCTGCAGTGACTCAACAAAGCGAT | |
| | ATGAACATCTTGCGATGGTATCCTGCTGATAGTTTTTACTGTACAAACACCTG | |
| | TGTAGCTCCTTCTAGCATTTTTAAGTTATTCACACCTCAAGGGGAGGGATAAA | |
| | TTAAATAAATTCCAAAAGCGAAGATCGAGAAACTAAATTAAAATTCCAAAAAC | |
| | GAAGTTGGAACACAACCCCCCGAAAAAAAACAACAAACAAAAAACCCAACAAA | |
| | ATAAACAAAAACAAAATAAATATATAACTACCAGTATCTGACTAAAAGTTCAA | |
| | ATACTCGTACTTACAACAAATAGAAATGAGCCGGCCAAAATTCTGCAGAAAAA | |
| | AATTTCAAACAAGTACTGGTATAATTAAATTAAAAAACACATCAAAGTATCAT | |
| | AACGTTAGTTATTTTATTTTATTTAATAAAAGAAAACAACAAGATGGGCTCAA | |
| | AACTTTCAACTTATACGATACATACCAAATAACAATTTAGTATTTATCTAAGT | |
| | GCTTTTCGTAGATAATGGAATACAAATGGATATCCAGAGTATACACATGGATA | |
| | GTATACACTGACACGACAATTCTGTATCTCTTTATGTTAACTACTGTGAGGCA | |
| | TTAAATAGAGCTTGATATATAAAATGTTACATTTCACAGTCTGAACTTTTGCA | |
| | GATTACCTAATTTGGTAAGATATTAATTATGAACTGAAAGTTGATGGCATCCC | |
| | TAAATTTGATGAAAGATGAAATTGTAAATGAGGTGGTAAAAGAGCTACAGTCG | |
| | TTTTGTTTTGAGATACCATCATCTCTAACGAAATATCTATTAAAAATCTCAGT | |
| | GTGATCATGAGTCATTGCCATCCTGGAAAATGTCATCATGGCTGATATTTCTA | |
| | ACTGTTTACTTGAGATAAATATATATTTACAAGAACTTCCCTTGAAATTAATT | |
| | TAGATATAAAATGTTTGCGGGCAAGTTACTACGAGGAATAAATTATATCTAGA | |
| | GGTTCCGCTTCCTCGCTCACTGACTCGCTGCGCTCGGTCGTTCGGCTGCGGCG | |
| | AGCGGTATCAGCTCACTCAAAGGCGGTAATACGGTTATCCACAGAATCAGGGG | |
| | ATAACGCAGGAAAGAACATGTGAGCAAAAGGCCAGCAAAAGGCCAGGAACCGT | |
| | AAAAAGGCCGCGTTGCTGGCGTTTTTCCATAGGCTCCGCCCCCCTGACGAGCA | |
| | TCACAAAAATCGACGCTCAAGTCAGAGGTGGCGAAACCCGACAGGACTATAAA | |
| | GATACCAGGCGTTTCCCCCTGGAAGCTCCCTCGTGCGCTCTCCTGTTCCGACC | |
| | CTGCCGCTTACCGGATACCTGTCCGCCTTTCTCCCTTCGGGAAGCGTGGCGCT | |
| | TTCTCATAGCTCACGCTGTAGGTATCTCAGTTCGGTGTAGGTCGTTCGCTCCA | |
| | AGCTGGGCTGTGTGCACGAACCCCCCGTTCAGCCCGACCGCTGCGCCTTATCC | |
| | GGTAACTATCGTCTTGAGTCCAACCCGGTAAGACACGACTTATCGCCACTGGC | |
| | AGCAGCCACTGGTAACAGGATTAGCAGAGCGAGGTATGTAGGCGGTGCTACAG | |
| | AGTTCTTGAAGTGGTGGCCTAACTACGGCTACACTAGAAGGACAGTATTTGGT | |
| | ATCTGCGCTCTGCTGAAGCCAGTTACCTTCGGAAAAAGAGTTGGTAGCTCTTG | |
| | ATCCGGCAAACAAACCACCGCTGGTAGCGGTGGTTTTTTTGTTTGCAAGCAGC | |
| | AGATTACGCGCAGAAAAAAAGGATCTCAAGAAGATCCTTTGATCTTTTCTACG | |
| | GGGTCTGACGCTCAGTGGAACGAAAACTCACGTTAAGGGATTTTGGTCATGAG | |
| | ATTATCAAAAAGGATCTTCACCTAGATCCTTTTAAATTAAAAATGAAGTTTTA | |
| | AATCAATCTAAAGTATATATGAGTAAACTTGGTCTGACAGTTACCAATGCTTA | |
| | ATCAGTGAGGCACCTATCTCAGCGATCTGTCTATTTCGTTCATCCATAGTTGC | |
| | CTGACTCCCCGTCGTGTAGATAACTACGATACGGGAGGGCTTACCATCTGGCC | |
| | CCAGTGCTGCAATGATACCGCGAGACCCACGCTCACCGGCTCCAGATTTATCA | |
| | GCAATAAACCAGCCAGCCGGAAGGGCCGAGCGCAGAAGTGGTCCTGCAACTTT | |
| | ATCCGCCTCCATCCAGTCTATTAATTGTTGCCGGGAAGCTAGAGTAAGTAGTT | |
| | CGCCAGTTAATAGTTTGCGCAACGTTGTTGCCATTGCTGCAGGCATCGTGGTG | |
| | TCACGCTCGTCGTTTGGTATGGCTTCATTCAGCTCCGGTTCCCAACGATCAAG | |
| | GCGAGTTACATGATCCCCCATGTTGTGCAAAAAAGCGGTTAGCTCCTTCGGTC | |
| | CTCCGATCGTTGTCAGAAGTAAGTTGGCCGCAGTGTTATCACTCATGGTTATG | |
| | GCAGCACTGCATAATTCTCTTACTGTCATGCCATCCGTAAGATGCTTTTCTGT | |
| | GACTGGTGAGTACTCAACCAAGTCATTCTGAGAATAGTGTATGCGGCGACCGA | |
| | GTTGCTCTTGCCCGGCGTCAACACGGGATAATACCGCGCCACATAGCAGAACT | |
| | TTAAAAGTGCTCATCATTGGAAAACGTTCTTCGGGGCGAAAACTCTCAAGGAT | |
| | CTTACCGCTGTTGAGATCCAGTTCGATGTAACCCACTCGTGCACCCAACTGAT | |
| | CTTCAGCATCTTTTACTTTCACCAGCGTTTCTGGGTGAGCAAAAACAGGAAGG | |
| | CAAAATGCCGCAAAAAAGGGAATAAGGGCGACACGGAAATGTTGAATACTCAT | |
| | ACTCTTCCTTTTTCAATATTATTGAAGCATTTATCAGGGTTATTGTCTCATGA | |
| | GCGGATACATATTTGAATGTATTTAGAAAAATAAACAAATAGGGGTTCCGCGC | |
| | ACATTTCCCCGAAAAGTGCCACCTGACGTCTAAGAAACCATTATTATCATGAC | |
| | ATTAACCTATAAAAATAGGCGTATCACGAGGCCCTTTCGTCTGGCCTAGGAAG | |
| | CGACTTCCAATCGCTTTGCATATCCAGTACCACACCCACAGGCGTTTGTGCTA | |
| | CTCTACTGATAGCAATAGATGCGTCATAATTGGTTGGCCCGCTGAGCCTCCAC | |
| | AGGATACTATTGCACATACCCTGGTCATGTGCAGATCAGCTCATTTGTGGAGA | |
| | CTCTGGAGTAACTTAGACGACGCCTGGTTCAATTGCCGCAATGTGCGCCCACG | |
| | CAGATAATGTATTGAGGGGTGGAGCGCCTCTTGGGGACTTGCTGTACTTGTAC | |
| | GGGATATTAAACGCACTCAGCAAGACCATGACGTAAAACACACCTACTGTACG | |
| | ATACGTACTGTAGGTATTGTACTCGTACCCGGTACTACAAATAGTACGATACT | |
| | ATACGGAGTGTATTTGTACCTTGATATACGACTGGCGGAGTGAAGAGAAGGAG | |
| | TTGAACAAGACCAGATGGGGATATCAGCCCCAGTGCTTTGTATTACAAGTACG | |
| | AGTACTTAATAGATACTGTAAGGCTATTGATACGGATGGCAGTAAGTCATTGA | |
| | GTAAGCAATTGTGGCCCAGCATCTCCCCTACGTACTTGTACCATACCCCATGG | |
| | AGACACCAATGGTCTTTCACGCACACTGTCGTGTGCTGTATCGCAGAATCGGG | |
| | TGTCCAACCAAATGCCGTTACCCCCACGTCACAGCCGATAGACAGATACACCA | |
| | TCAATACCAGCAGGTTGTATCATGCGGTTGGCTGAAGGTAAGCTGATTGGTCT | |
| | AAAAACTGTAGCTGTCCTAATTCAACGAGCGCTATTTGGGGCCAACCACCTCG | |
| | GCCAAGCGGCCTTTAATCTGCGTGCCCCAGAGGCGTCTAATGAGGCTCTGGCC | |
| | GCCACTGTAGGAGTGTTTCTCTGTGCGCACACGCAGTTTTGAGTTTGGGCGAC | |
| | TTTCCCTTTTTCCCAATTGCGTACACACACAGCTCCGAGCTAAGCGCTGTCCT | |
| | TGAACCTTCTCCCTCTTTTCCCTCTTTTTCTCTTCCCCTTCCCCTCCTCCACA | |
| | TTAAGGCCAAATCCTGAATTGCACCAACTAGTACAACGACAACAATGGACAAG | |
| | AAGTACTCCATCGGTTTGGACATTGGTACTAACTCTGTCGGCTGGGCCGTCAT | |
| | CACCGACGAGTACAAGGTTCCCTCCAAGAAGTTCAAGGTCCTTGGCAACACCG | |
| | ACCGACACTCTATCAAGAAGAACCTGATCGGTGCTCTGCTGTTCGACTCTGGC | |
| | GAGACTGCCGAGGCCACCCGACTGAAGCGAACCGCTCGACGCCGATACACCCG | |
| | ACGAAAGAACCGAATCTGTTACCTCCAGGAGATCTTCAGCAACGAGATGGCTA | |
| | AGGTCGACGACTCCTTCTTCCACCGACTCGAGGAGTCTTTCCTGGTCGAAGAG | |
| | GATAAGAAGCACGAGCGACACCCCATCTTCGGCAACATTGTTGATGAGGTTGC | |
| | CTACCATGAGAAGTACCCCACCATCTACCACCTCCGAAAGAAGCTCGTCGACT | |
| | CCACTGACAAGGCTGACCTCCGACTCATCTACCTTGCTCTCGCCCACATGATC | |
| | AAGTTCCGAGGTCACTTCCTCATTGAGGGTGATCTCAACCCCGACAACTCCGA | |
| | CGTTGACAAGCTGTTCATCCAGCTCGTCCAGACCTACAACCAGCTCTTTGAGG | |
| | AGAACCCTATCAACGCTTCTGGTGTTGACGCCAAGGCCATTCTCTCCGCCCGA | |
| | CTCTCTAAGTCCCGACGACTCGAGAACCTCATTGCCCAGCTGCCCGGCGAGAA | |
| | GAAGAACGGCCTCTTCGGTAACCTGATTGCTCTCTCTCTTGGTCTGACCCCCA | |
| | ACTTCAAGTCCAACTTTGACCTCGCCGAGGACGCCAAGCTCCAGCTGTCCAAG | |
| | GACACCTACGATGACGATCTGGACAACCTCCTGGCCCAGATCGGTGACCAGTA | |
| | CGCCGATCTCTTCCTTGCCGCCAAGAACCTCTCCGACGCCATCCTGCTCTCCG | |
| | ACATCCTCCGAGTCAACACCGAGATTACCAAGGCTCCTCTGTCTGCCTCTATG | |
| | ATCAAGCGATACGACGAGCACCACCAGGATCTCACTCTTCTCAAGGCTCTCGT | |
| | CCGACAGCAGCTCCCCGAGAAGTACAAGGAGATTTTCTTTGACCAGTCCAAGA | |
| | ACGGTTACGCTGGCTACATTGACGGTGGTGCTTCCCAGGAAGAGTTTTACAAG | |
| | TTCATCAAGCCTATTCTGGAGAAGATGGACGGTACCGAGGAGCTGCTCGTCAA | |
| | GCTCAACCGAGAGGACCTCCTTCGAAAGCAGCGAACCTTCGATAACGGCTCCA | |
| | TCCCCCACCAGATCCACCTGGGTGAGCTCCACGCCATTCTCCGAAGACAAGAG | |
| | GACTTCTACCCCTTCCTAAAGGATAACCGAGAGAAGATCGAGAAGATTCTCAC | |
| | CTTCCGAATCCCCTACTACGTCGGTCCCCTCGCTCGAGGTAACTCCCGATTTG | |
| | CTTGGATGACCCGAAAGTCCGAGGAGACTATCACCCCCTGGAACTTTGAAGAG | |
| | GTAGTCGACAAGGGTGCCTCCGCCCAGTCTTTCATTGAGCGGATGACCAACTT | |
| | CGATAAGAACCTCCCCAACGAGAAGGTCCTTCCCAAGCACTCTCTCCTCTACG | |
| | AGTACTTCACCGTCTACAACGAGCTGACCAAGGTCAAGTACGTTACCGAGGGC | |
| | ATGCGAAAGCCCGCTTTCCTCTCTGGTGAGCAGAAGAAGGCCATTGTCGACCT | |
| | CCTGTTCAAGACTAACCGAAAAGTCACCGTCAAGCAGCTCAAGGAAGACTACT | |
| | TCAAGAAGATTGAGTGCTTCGACTCCGTCGAGATTTCCGGTGTCGAGGACCGA | |
| | TTCAACGCCTCCCTCGGCACCTACCACGATCTTCTGAAGATCATCAAGGACAA | |
| | GGACTTTCTTGATAACGAGGAGAACGAGGACATTCTCGAGGACATCGTCCTCA | |
| | CCCTCACCCTTTTCGAGGATCGAGAGATGATCGAGGAGCGACTCAAGACCTAC | |
| | GCCCATCTCTTCGACGACAAGGTCATGAAGCAACTCAAGCGACGACGATACAC | |
| | TGGCTGGGGCCGACTTTCCCGAAAGCTCATCAACGGCATCCGAGACAAGCAGT | |
| | CTGGCAAGACCATCCTGGACTTCCTGAAGTCCGACGGTTTCGCCAACCGAAAC | |
| | TTCATGCAGCTCATCCACGACGACTCTCTTACCTTCAAAGAGGATATCCAGAA | |
| | GGCCCAGGTTTCTGGCCAGGGCGACTCCCTCCACGAGCACATTGCCAACCTCG | |
| | CCGGATCCCCCGCCATCAAAAAGGGTATCCTCCAGACCGTCAAGGTTGTCGAC | |
| | GAACTCGTGAAGGTCATGGGCCGACACAAGCCCGAGAACATCGTTATCGAGAT | |
| | GGCCCGAGAGAACCAGACCACCCAGAAGGGTCAGAAGAACTCCCGAGAGCGAA | |
| | TGAAGCGAATCGAAGAGGGTATCAAGGAGCTCGGTTCCCAGATTCTCAAGGAG | |
| | CACCCCGTCGAGAACACCCAGCTCCAGAACGAGAAACTCTACCTGTACTACCT | |
| | CCAGAATGGCCGAGACATGTACGTTGACCAGGAGCTCGACATCAACCGACTCT | |
| | CCGACTACGACGTCGACCACATTGTTCCTCAGTCCTTCCTCAAGGACGACTCC | |
| | ATCGACAACAAGGTTCTGACCCGATCTGACAAGAACCGAGGTAAGTCCGACAA | |
| | CGTTCCCTCCGAAGAGGTCGTTAAGAAGATGAAGAACTACTGGCGACAGCTTC | |
| | TCAACGCCAAACTGATCACCCAGCGAAAGTTTGACAACCTCACCAAGGCCGAG | |
| | CGAGGTGGTCTGTCCGAGCTGGACAAGGCCGGCTTCATTAAGCGACAGCTGGT | |
| | CGAGACTCGACAGATCACCAAGCACGTCGCCCAGATCCTCGACTCCCGAATGA | |
| | ACACCAAGTACGACGAGAACGACAAGCTCATCCGGGAGGTCAAGGTCATCACC | |
| | CTGAAGTCTAAGCTTGTCTCCGACTTCCGAAAGGACTTCCAGTTCTACAAGGT | |
| | CCGAGAGATCAACAACTACCACCACGCCCACGACGCCTACCTCAACGCCGTTG | |
| | TTGGTACCGCCCTCATCAAGAAGTATCCCAAGCTCGAGTCCGAGTTCGTTTAC | |
| | GGCGACTACAAGGTTTACGATGTCCGAAAGATGATTGCCAAGTCCGAGCAGGA | |
| | GATCGGTAAGGCCACCGCCAAGTACTTTTTCTACTCCAACATCATGAATTTCT | |
| | TCAAGACCGAGATCACTCTCGCCAACGGTGAGATTCGAAAGCGACCCCTGATT | |
| | GAGACTAATGGTGAGACTGGTGAGATCGTCTGGGATAAGGGCCGAGACTTCGC | |
| | CACCGTCCGAAAGGTCCTGTCCATGCCCCAGGTCAACATTGTCAAGAAGACCG | |
| | AGGTCCAGACCGGTGGCTTCTCCAAGGAGTCCATTCTCCCCAAGCGAAACTCC | |
| | GACAAACTCATCGCCCGTAAGAAGGACTGGGATCCGAAGAAGTACGGTGGTTT | |
| | CGATTCTCCCACCGTTGCCTACTCCGTCCTCGTTGTTGCTAAAGTCGAGAAGG | |
| | GTAAGTCTAAGAAACTCAAGTCCGTGAAGGAGCTACTCGGTATCACCATCATG | |
| | GAGCGATCTTCTTTTGAGAAGAACCCCATTGACTTCCTCGAGGCCAAGGGTTA | |
| | CAAAGAGGTCAAGAAGGACCTGATTATCAAGCTGCCCAAGTACTCCCTCTTTG | |
| | AGCTCGAGAACGGCCGAAAGCGAATGCTGGCTTCCGCTGGTGAGCTGCAGAAG | |
| | GGCAACGAGCTCGCTCTGCCCTCCAAGTACGTCAACTTCCTCTACCTGGCCTC | |
| | CCACTACGAGAAGCTCAAGGGCTCCCCCGAGGACAACGAGCAGAAGCAGCTGT | |
| | TCGTTGAGCAGCACAAGCACTACCTCGACGAGATCATCGAGCAGATCTCCGAG | |
| | TTCTCCAAGCGAGTCATCCTCGCTGACGCCAACCTTGATAAGGTTCTCTCTGC | |
| | TTACAACAAGCACCGGGACAAGCCCATCCGAGAGCAGGCCGAGAATATCATCC | |
| | ACCTCTTCACTCTCACCAACCTCGGCGCTCCTGCTGCCTTCAAGTACTTCGAC | |
| | ACCACCATTGACCGAAAGAGGTACACCTCCACCAAGGAAGTCCTCGACGCCAC | |
| | CCTGATCCACCAGTCCATCACCGGCCTCTACGAAACCCGAATCGACCTCTCCC | |
| | AGCTCGGCGGTGACTCTCGAGCCGACCCCAAGAAGAAGCGAAAAGTCTAAATA | |
| | TCCGAAGATCAAGAGCGAAGCAAGTTGTAAGTCCAGGACATGTTTCCCGCCCA | |
| | CGCGAGTGATTTATAACACCTCTCTTTTTTGACACCCGCTCGCCTTGAAATTC | |
| | ATGTCACATAAATTATAGTCAACGACGTTTGAATAACTTGTCTTGTAGTTCGA | |
| | TGATGATCATATGATTACATTAATAGTAATTACTGTATTTGATATATATACTA | |
| | ATTACAATAGTACATATTAGAACATACAATAGTTAGTGCCGTGAAGTGGCTTA | |
| | AAATACCGCGAGTCGATTACGTAATATTATTACCTCTTGCCCATCGAACGTAC | |
| | AAGTACTCCTCTGTTCTCTCCTTCCTTTGCTTTGTGCACGAAGAACTGCGGTC | |
| | AGGTGACACAACTTTTTCCATCTCAGGGTGTGTCGCGTGTGCTTCATCCAAAC | |
| | TTTAGTTGGGGTTCGGGTTCGCGCGAGATGATCACGTGCCCTGATTTGGTGTC | |
| | GTCCCCCGTCGCGCTGCGCACGTGATTTATTTATTTCCGGTGGCTGCTGTCTA | |
| | CGCGGGGCCTTCTCTGCCCTTCTGTTTCAACCTTCGGGCGGTTCTCGTAACCA | |
| | GCAGTAGCAATCCATTTCGAAACTCAAAGAGCTAAAAACGTTAAACCTCAGCA | |
| | GTCGCTCGACGAATGGGCTGCGGTTGGGAAGCCCACGAGGCCTATAGCCAGAG | |
| | CCTCGAGTTGACAGGAGCCCAGACGCCTTTTCCAACGGCAACTTTTATATAAA | |
| | ATGGCAATGTATTCATGCAATTGCGGCCGTGTCAGGTTGGAGACACTGGACCA | |
| | CACTCTCCATTGCTTCCTGAGGAGATGGATCATTGCTAGTGCATCTACGCGCA | |
| | GCAATCCCGCAAGCTCGACAACCGTAGATGGGCTTTGGTGGGCCAATCAATTA | |
| | CGCAACCCGCACGTTAAATTGTATGAGGAAGGAAGGCCACGGTACAAAGTGGG | |
| | TGGTCTTCACCCAGTGGTTGTTGGTGGCGTCATGCAGACCATGGCCGCCAGTG | |
| | TGCTGGAATTGAATATTTACCGTTCGTATAATGTATGCTATACGAAGTTATAC | |
| | CGGTCTCGTAGTGTTCACGTTCAGTTCACGGTGAGCTTAAAACTATCTTCAAG | |
| | AAGAGATTTGAGACCTGATTTATACTTGCAGCAATGTTTACTTCTTATCGCGA | |
| | TACACGAATGTGATACGGATCAAAGTAAGCAGGACTACGATAAGATAACGAAT | |
| | GCGGTGCAGTCCATGTCGATTAGGTATAGATACATTTATTTTGTGTTATGTTA | |
| | CATTTTGGGGGGATACTGTCCTACTTGTAGTACCTACTTGTAGTGGCGCGTTA | |
| | GGGGCAGGGCATGCTCATGTAGAGCGCCTGCCGCTCGCCGTCCGAGGCGGTGC | |
| | CGTCGTACAGGGCGGTGTCCAGGCCGCAGAGGGTGAACCCCATCCGCCGGTA | |
| MB9523 | GCGGAGCCTAGGCCGGCCAGCGTGGCGCGTGGCCATATTGGCCAGCAGCTTAC | 8 |
| | TACAGCTCAGCCAAAGTGGATCATGATGGACGGAAATATCGGACTGGAGGCCA | |
| | AAAAGGAGGTGCTCAAATACGCACGGGACAAGTCTGCACAGGTGGCATTCGAA | |
| | CCCACGTCTGTCCCCAAAGCCGCTGCTCTTTCCGAGCTCAATCTGCCCGTTTA | |
| | CCCCAACAACTCCATTGCCTTAGCCACCCCCAACACTGCAGAGCTCGAGGCCA | |
| | TGTTCGAGGCATTCCACGAGAAGGGCCGATTTGACGTTGACGACTGGTTTCCA | |
| | GTCATAGACGGTCTTGCTCTCGGAGCTGACTTTAGAAACGGAGCCACCATGTT | |
| | GTCGCACCAACATCGTGGACTCAAAGCTATTCTCGAACAAGGAACTCTGGCCC | |
| | AGGCAATCCACATGCTACCATACATCCCAACTTTGATCATCAAGGGCGGAGCC | |
| | AACGGCGTCGTCGTCTTCCAACTCATTGATGATATCGAGTCTGCCATTCATTC | |
| | ACAGCGTTCTGCTTCCAATAAAACGCCTGGCCTGTTCCAGAAGGGCAACGCTG | |
| | CGAGTGGAAACACCAAGGTCGGCGTCTACATGCAGTACTTTGAGCCCGAAGAA | |
| | GTGGGCAGTCAGTCGATTGTAAGCGTGACTGGAGCTGGAGACACTCTGTTTGG | |
| | AACCCTGGCCATGGAGATTGTCAAGGACGAGTCCTGGTTGAACGATATGGGAG | |
| | ACAAGAAGAGTGCAGTTGTTTCTCGAGCCATGAACAATGCTGTGAAGACTATT | |
| | CAGAGTAAGGACGCTGTCTGCAAGAGCATCCTTTAAGTGATTTGCCATGCTTT | |
| | CTCTTCTTCCACGATGTAAATACTTATTTTACACACTACTGTGCAGTAGCAAA | |
| | TACAGAACAAGAGTTGTCGCCTATTGACAGTACAGTACGAGTAGTGTATGTAC | |
| | AGTAGTTATACAATATCTATGTGAAATTCGTCGGCAGCTTTCGGCTGATGAAC | |
| | TACGAGTTCTTCGTAATAAATCATCAACGTAATAAGCTTGGTACCAGAGACGG | |
| | GTTGGCGGCGTATTTGTGTCCCAAAAAACAGCCCCAATTGCCCCAATTGACCC | |
| | CAAATTGACCCAGTAGCGGGCCCAACCCCGGCGAGAGCCCCCTTCACCCCACA | |
| | TATCAAACCTCCCCCGGTTCCCACACTTGCCGTTAAGGGCGTAGGGTACTGCA | |
| | GTCTGGAATCTACGCTTGTTCAGACTTTGTACTAGTTTCTTTGTCTGGCCATC | |
| | CGGGTAACCCATGCCGGACGCAAAATAGACTACTGAAAATTTTTTTGCTTTGT | |
| | GGTTGGGACTTTAGCCAAGGGTATAAAAGACCACCGTCCCCGAATTACCTTTC | |
| | CTCTTCTTTTCTCTCTCTCCTTGTCAACTCACACCCGAAATCGTTAAGCATTT | |
| | CCTTCTGAGTATAAGAATCATTCGCTAGCCACAAAAATGGGTGATCTCGATGC | |
| | CCGAGGAACCTCTGCTCACCCCGAGCTCTCTGAGCGACCTTCTATTATGCCTT | |
| | CTATGTCTGATATTCAGGACCCTTCTGGTGATGACAAGGCTACTCCCCGAGGA | |
| | TCTGCTGCTGGTCTGCCCCAGCTTGAGCTTGCTGGACACGCCCGACGACTTGG | |
| | CCACCTTGAGAACTTCTTTGCTGTCCAGGCTCGACAGCAGATTTACTCTTCTT | |
| | TTGCTGTTTTTTGTGAGTTTGACACTGCTTGTTCTCTCGCTCAGCTTGCTTCT | |
| | GCTGTGCGAAACGTTTGTCTTTCTAACCCCCTTCTCCTTCACACTGTTGAGCC | |
| | TAAGCACCCTGACATCGCTGGATTCTACCACTCTGACGAGTACCTTTCCCGAC | |
| | CTTGGCCTCAGCACGATTACATGCGAGTTCTTCGAGAGGTTCACGTCGCTGAC | |
| | GTTGTTATGAACGGACAGAAGGAGCACGCTCACGTTGTTCGAGATGCTGTTGA | |
| | CGTTTTTCAGGCTCACGGAAACCAGGTTACTTCTGAGCTCCTTGAGCTTATGA | |
| | CTCAGATTGAGATTCCTCACGCTTCTCAGACTCGACCCTCTTGGCGACTTCTC | |
| | TGTTTTCCCCACGGAGAGGCTAACCGATGGCGAACCTTTGCTTTTGTTTCTAA | |
| | CCACTGTTCTTCTGATGGTCTTTCTGGTCTTAACTTCTTTCGAGATCTCCAGA | |
| | AGGAGCTTGCTCACGGCCCCACCTCTGGTGCTCCTGGTGCCCCCGGAGCTTCC | |
| | GGAGTTATTTTCGATTACGCTCAGGACGCTGCTACCCTGCCCAAGCTGCCCCC | |
| | TCCCATTGATCAGAAGCTCGATTACCGACCTTCTAAGAAGGCTCTTCTCGGCC | |
| | TTCTCGCTGGCAAGTTCGTTCGAGAGAAGCTCGGTTACGTTTCTGCTGCTCCT | |
| | CCCACTACCCCTACCTCTGACCTTGCTCACCCTGAGGGTCACCAGTACTACTG | |
| | TTACCTTGTTAACGTTCCCACTTCTTCTGTTGCCCACATTAAGACTCAGGTGC | |
| | GAGAGAACGTTCCTCACAAGTGTACTCTCACTCCCTTTCTCCAGGCTTGTTGG | |
| | CTTGTTTCTCTGTTCAAGTACGGTCGAGTTTTTTCTGGTTCTTGGCTTGAGCG | |
| | ATACACCGATGTTCTTGTTGCTATGAACACTCGACAGCTTCTCCCCGAGGACC | |
| | TTGAGCTTCAGCGACAGTACCGATACGGTTCTAACATTGGAGCTGTTCGATAC | |
| | AACTACCCTATTGCTCCCCTTGACGTTCGAGATAACGATCAGAAGTTCTGGTC | |
| | CCTTGTTGAGTCTTACCGACTTGCCCTTTCTGATGCCCGAGATAAGAACGATT | |
| | ACCTTTACGCTCTTGGTGCTCTTATGCTCCCTGAGATTTACGAGAAGAAGAAC | |
| | GTTGATGCTGTTGTTAACGATACCATTCTTAACCAGCGACGACAGGGAACCCT | |
| | TCTTTCTAACGTTGGTTACGTTCGAGATGAGCAGCCCACTGCTTTTGCTATTA | |
| | AGAACCACGTTTTTTCTCAGGGAGTTGGAGCTAACCGAAACGCTTTTGTTCTT | |
| | AACATTTGTGCTACCGATCAGGGTGGTCTTAACATCGCTATTTCTATTGCTAA | |
| | GGGAACCCTTGCTTCTCGACAGGAGGGACAGGAGCTTTGTGATATTTTTAAGT | |
| | CTACTCTCCTTCGATTTTAAACGCGTCTATCCGAAGATCAAGAGCGAAGCAAG | |
| | TTGTAAGTCCAGGACATGTTTCCCGCCCACGCGAGTGATTTATAACACCTCTC | |
| | TTTTTTGACACCCGCTCGCCTTGAAATTCATGTCACATAAATTATAGTCAACG | |
| | ACGTTTGAATAACTTGTCTTGTAGTTCGATGATGATCATATGATTACATTAAT | |
| | AGTAATTACTGTATCTGTACCTGCTGTGGACCACGCACGGCGGAACGTACCGT | |
| | ACAAATATTTTCTTGCTCACATGACTCTCTCTCGGCCGCGCACGCCGGTGGCA | |
| | AATTGCTCTTGCATTGGCTCTGTCTCTAGACGTCCAAACCGTCCAAAGTGGCA | |
| | GGGTGACGTGATGCGACGCACGAAGGAGATGGCCCGGTGGCGAGGAACCGGAC | |
| | ACGGCGAGCCGGCGGGAAAAAAGGCGGAAAACGAAAAGCGAAGGGCACAATCT | |
| | GACGGTGCGGCTGCCACCAACCCAAGGAGGCTATTTTGGGTCGCTTTCCATTT | |
| | CACATTCGCCCTCAATGGCCACTTTGCGGTGGTGAACATGGTTTCTGAAACAA | |
| | CCCCCCAGAATTAGAGTATATTGATGTGTTTAAGATTGGGTTGCTATTTGGCC | |
| | ATTGTGGGGGAGGGTAGCGACGTGGAGGACATTCCAGGGCGAATTGAGCCTAG | |
| | AAAGTGGTACCATTCCAACCGTCTCAGTCGTCCGAATTGATCGCTATAACTAT | |
| | CACCTCTCTCACATGTCTACTTCCCCAACCAACATCCCCAACCTCCCCCACAC | |
| | TAAAGTTCACGCCAATAATGTAGGCACTCTTTCTGGGTGTGGGACAGCAGAGC | |
| | AATACGGAGGGGAGATTACACAACGAGCCACAATTGGGGAGATGGTAGCCATC | |
| | TCACTCGACCCGTCGACTTTTGGCAACGCTCAATTACCCACCAAATTTGGGCT | |
| | GGAGTTGAGGGGACCGTGTTCCAGCGCTGTAGGACCAGCAACACACACGGTAT | |
| | CAACAGCAACCAACGCCCCCGCTAATGCACCCAGTACTGCGCAGGTGTGGGCC | |
| | AGGTGCGTTCCAGATGCGAGTTGGCGAACCCTAAGCCGACAGTGTACTTTTTG | |
| | GGACGGGCAGTAGCAATCGTGGGCGTAGACCCCGGTGTATATAAAGGGGTGGA | |
| | GAGGACGGATTATTAGCACCAACACACACACTTATACTACATGCTAGCCACAA | |
| | AAATGCTCTCTTTCTTCTGGCGAAACGGTATCGAGACTCCCGAGCCCCTCAAG | |
| | GCTGACGTTTCCGGCTCTATCCCTCCCTGGCTTCAGGGAACCCTTCTCCGAAA | |
| | CGGTCCTGGTCTGTTCTCCGTTGGCAACACTTCCTACAAGCACTGGTTCGATG | |
| | GTATGGCTCTCATTCACTCCTTCACCTTTAAGGATGGTGAGGTTTTTTACCGA | |
| | TCTAAGTACCTGAAGTCTGAGACTTACAAGAAGAACATCGCTGCCGACCGAAT | |
| | CGTTGTGTCTGAGTTCGGAACCATGGTGTACCCCGATCCCTGCAAGAACATTT | |
| | TCTCCCGAGCCTTCTCTTACATGATGAACGCCATTCCTGACTTTACCGATAAC | |
| | AACCTCATTAACATCATTAAGTACGGTGAGGATTACTACGCCTCCTCTGAGGT | |
| | CAACTACATCAACCAGATTGACCCCCTGACCCTTGAGACTCTCGGACGAACTA | |
| | ACTACCGAAACCACATTGCCATCAACCTTGCCACTGCTCACCCTCACTACGAC | |
| | GAGGAGGGTAACACTTACAACATGGGCACTGCTATTATGAACCTCGGTCGACC | |
| | CAAGTACGTGATTTTCAAGGTGCCCGCCAACACCTCTGATAAGGAGAACAAGA | |
| | AGCCTGCCCTCTCTGAGGTGGAGCAGGTTTGCTCCATTCCCATCCGACCCTCC | |
| | CTTTACCCTTCTTACTTCCACTCTTTTGGCATGACTGAGAACTACATCATCTT | |
| | CGTTGAGCAGGCCTTCAAGCTGGACATCGTCAAGCTGGCTACTGCTTACTTCC | |
| | GAGATATTAACTGGGGATCTTGCCTTAAGTTCGACCAGGATGACATTAACGTG | |
| | TTCCACCTGGTCAACAAGAAGACTGGTAAGGCTGTGTCCGTGAAGTACTACAC | |
| | TGACCCCTTTGTTACCTTCCACCACATCAACGCTTACGAGGACGATGGCCACG | |
| | TCGTCTTCGATCTCATTACTTACAAGGACTCTAAGCTGTACGATATGTTCTAC | |
| | ATTCAGAACATGAAGCAGGACGTCAAGCGATTTATTGAGACTAACAAGGACTT | |
| | CGCTCAGCCCGTGTGCCAGCGATTTGTCCTTCCCGTCAACGTTGATAAGGAGA | |
| | CTCCTCAGGACATCAACCTTGTCAAGCTGCAGGACACCACTGCCACTGCTGTC | |
| | CTGAAGGAGGACGGCTCTGTCTACTGCACCCCTGACATCATTTTTAAGGGTCT | |
| | TGAGCTCCCTGCTATCAACTACAAGTTTAACTCTAAGAAGAACCGATACTTCT | |
| | ACGGCACCCGAGTGGAGTGGTCCCCTTACCCTAACAAGGTCGCTAAGGTGGAC | |
| | GTTGTTACTCGAACCCACAAGATTTGGACTGAGGAGGAGTGTTACCCTTCTGA | |
| | GCCTGTCTTTATTGCCTCCCCTGACGCCGTTGATGAGGATGACGGTGTGATTC | |
| | TTTCTTCTGTGGTTTCTTTCAACCCCCAGCGACCCCCTTTCCTGGTTGTCCTC | |
| | GATGCTAAGTCCTTCAAGGAGATTGCTCGAGCTACCATCGATGCCTCTATTCA | |
| | CATGGACCTTCACGGCCTTTTCATCCACGACAAGTCTACCTAAGTTTTTTGAT | |
| | CAATGATCCAATGGCTTTCACATACCCCCCCACGCCTATAATTAAAACACAGA | |
| | GAAATATAATCTAACTTAATAAATATTACGGAGAATCTTTCGAGTGTTCAGCA | |
| | GAAATATAGCCATTGTAACAAAAGCCGGCTATCGACCGCTTTATCGAAGAATA | |
| | TTTCCCGCCCCCCAGTGGCCAAACGATATCGGTCAGAAGGGGCAGCTCTAAAC | |
| | GAAGAACTGCGGTCAGGTGACACAACTTTTTCCATCTCAGGGTGTGTCGCGTG | |
| | TGCTTCATCCAAACTTTAGTTGGGGTTCGGGTTCGCGCGAGATGATCACGTGC | |
| | CCTGATTTGGTGTCGTCCCCCGTCGCGCTGCGCACGTGATTTATTTATTTCCG | |
| | GTGGCTGCTGTCTACGCGGGGCCTTCTCTGCCCTTCTGTTTCAACCTTCGGGC | |
| | GGTTCTCGTAACCAGCAGTAGCAATCCATTTCGAAACTCAAAGAGCTAAAAAC | |
| | GTTAAACCTCAGCAGTCGCTCGACGAATGGGCTGCGGTTGGGAAGCCCACGAG | |
| | GCCTATAGCCAGAGCCTCGAGTTGACAGGAGCCCAGACGCCTTTTCCAACGGC | |
| | AACTTTTATATAAAATGGCAATGTATTCATGCAATTGCGGCCGTGTCAGGTTG | |
| | GAGACACTGGACCACACTCTCCATTGCTTCCTGAGGAGATGGATCATTGCTAG | |
| | TGCATCTACGCGCAGCAATCCCGCAAGCTCGACAACCGTAGATGGGCTTTGGT | |
| | GGGCCAATCAATTACGCAACCCGCACGTTAAATTGTATGAGGAAGGAAGGCCA | |
| | CGGTACAAAGTGGGTGGTCTTCACCCAGTGGTTGTTGGTGGCGTCATGCAGAC | |
| | CATGCATTGGGGATAGCACAGGGTTGGGGTGTCTTGTGGACTCAATGGGTGAA | |
| | AGGAGATGGAAAAGGGCGGTGAAAAGTGGTAGAATCGAAATCCCTGACGTCAA | |
| | TTTATAAAGTAAAATGCGTTTCTGCCATTTTGCTCCCCTCCTTCTTTCGCAAT | |
| | CGCCTCCCCAAAAGTTGTCGTGGCAGTACACATGCTTGCATACAATGAAGCTA | |
| | ATCCGGCTTGCTCAGTAGTTGCTATATCCAGGCATGGTGTGAAACCCCTCAAA | |
| | GTATATATAGGAGCGGTGAGCCCCAGTCTGGGGTCTTTTCTCTCCATCTCAAA | |
| | ACTACTTTCTCACATGCTAGCCACAAAAATGACCACTAAGTACACTTCCGTTC | |
| | ACGAGTCTCCCAACGGCCCTGGTGACGCTCGACCCACCGCTTCCCAGATTATC | |
| | GACGATTACAACCTTGAGGGAGAGCTTTCTGGCAAGACTGTTCTCGTCACCGG | |
| | CTGTTCCTCTGGTATTGGTGTTGAGACTGCCCGAGCTATTTACCGAACTGGTG | |
| | CCACCCTTTACCTCACTGCCCGAGATGTCGATAAGGCCAAGACCGTTCTTCCC | |
| | GACCTTGTTGACACTTCCCGAGTCCACTTTCTCCACCTTGACCTTAACTCTCT | |
| | GGAGTCTGTTCGAGGTTTTGCTGAGAACTTCAAGTCTAAGTCCACTCAGCTTC | |
| | ACATTCTCATCGAGAACGCTGGCGTGATGGCCTGTCCCGAGGGCCGAACCGTC | |
| | GATGGTTTTGAGACTCAGTTTGGTATCAACCACCTTGCTCACTTTCTCCTCTT | |
| | TTACCTCCTCAAGGATACCCTTCTCAACTCTTCTACCCCCGCTTTCAACTCCC | |
| | GAGTTGTCATCCTCTCTTCTTGTGCTCACCAGGCTGGTTCCGTTCACCTTAAC | |
| | AACCTGTCTCTTGAGGGTGGATACGAGCCTTGGAAGTCTTACGGCCAGTCCAA | |
| | GACTGCCAACCTTTGGACTGCCCGAGAGATCGAGAAGCGATTTGGTGCTTCCG | |
| | GTATCCACTCTTGGGCTGTTCACCCCGGTTCCATCGCTACTGAGCTTCAGCGA | |
| | CACGTTTCCGACGAGCTTAAGCAGAAGTGGGCTGACGATAAGGAGGGTGCCAA | |
| | GCTGTGGAAGTCCACCGAGCAGGGTGCCGCCACCACTGTCCTTGCTGCTGTTT | |
| | CCCCTGAGCTTGAGGGTAAGGGCGGTCTTTACCTTGAGGATACCCAGGTTGCC | |
| | AAGCCCCCTGCCCGAGGAATGTTTGGTGTTGCTGACTGGGCTTACGATGAGGA | |
| | TGGCCCCTCTAAGCTCTGGGCCAAGTCTCTTGAGCTCCTTAAGCTCCAGTAAA | |
| | GGTTAGACTATGGATATGTAATTTAACTGTGTATATAGAGAGCGTGCAAGTAT | |
| | GGAGCGCTTGTTCAGCTTGTATGATGGTCAGACGACCTGTCTGATCGAGTATG | |
| | TATGATACTGCACAACCTGTGTATCCGCATGATCTGTCCAATGGGGCATGTTG | |
| | TTGTGTTTCTCGATACGGAGATGCTGGGTACAAGTAGCTAATACGATTGAACT | |
| | ACTTATACTTATATGAGGCTTGAAGAAAGCTGACTTGTGTATGACTTATTCTC | |
| | AACTACATCCCCAGTCACAATACCACCACTGCACTACCACTACACACTAGTGG | |
| | TGTGTTCTGTGGAGCATTCTCACTTTTGGTAAACGACATTGCTTCAAGTGCAG | |
| | CGGAATCAAAAAGTATAAAGTGGGCAGCGAGTATACCTGTACAGACTGTAGGC | |
| | GATAACTCAATCCAATTACCCCCCACAACATGACTGGCCAAACTGATCTCAAG | |
| | ACTTTATTGAAATCAGCAACACCGATTCTCAATGAAGGCACATACTTCTTCTG | |
| | CAACATTCACTTGACGCCTAAAGTTGGTGAGAAATGGACCGACAAGACATATT | |
| | CTGCTATCCACGGACTGTTGCCTGTGTCGGTGGCTACAATACGTGAGTCAGAA | |
| | GGGCTGACGGTGGTGGTTCCCAAGGAAAAGGTCGACGAGTATCTGTCTGACTC | |
| | GTCATTGCCGCCTTTGGAGTACGACTCCAACTATGAGTGTGCTTGGATCACTT | |
| | TGACGATACATTCTTCGTTGGAGGCTGTGGGTCTGACAGCTGCGTTTTCGGCG | |
| | CGGTTGGCCGACAACAATATCAGCTGCAACGTCATTGCTGGCTTTCATCATGA | |
| | TCACATTTTTGTCGGCAAAGGCGACGCCCAGAGAGCCATTGACGTTCTTTCTA | |
| | ATTTGGACCGATAGCCGTATAGTCCAGTCTATCTATAAGTTCAACTAACTCGT | |
| | AACTATTACCATAACATATACTTCACTGCCCCAGATAAGGTTCCGATAAAAAG | |
| | TTCTGCAGACTAAATTTATTTCAGTCTCCTCTTCACCACCAAAATGCCCTCCT | |
| | ACGAAGCTCGAGCTAACGTCCACAAGTCCGCCTTTGCCGCTCGAGTGCTCAAG | |
| | CTCGTGGCAGCCAAGAAAACCAACCTGTGTGCTTCTCTGGATGTTACCACCAC | |
| | CAAGGAGCTCATTGAGCTTGCCGATAAGGTCGGACCTTATGTGTGCATGATCA | |
| | AGACCCATATCGACATCATTGACGACTTCACCTACGCCGGCACTGTGCTCCCC | |
| | CTCAAGGAACTTGCTCTTAAGCACGGTTTCTTCCTGTTCGAGGACAGAAAGTT | |
| | CGCAGATATTGGCAACACTGTCAAGCACCAGTACAAGAACGGTGTCTACCGAA | |
| | TCGCCGAGTGGTCCGATATCACCAACGCCCACGGTGTACCCGGAACCGGAATC | |
| | ATTGCTGGCCTGCGAGCTGGTGCCGAGGAAACTGTCTCTGAACAGAAGAAGGA | |
| | GGACGTCTCTGACTACGAGAACTCCCAGTACAAGGAGTTCCTGGTCCCCTCTC | |
| | CCAACGAGAAGCTGGCCAGAGGTCTGCTCATGCTGGCCGAGCTGTCTTGCAAG | |
| | GGCTCTCTGGCCACTGGCGAGTACTCCAAGCAGACCATTGAGCTTGCCCGATC | |
| | CGACCCCGAGTTTGTGGTTGGCTTCATTGCCCAGAACCGACCTAAGGGCGACT | |
| | CTGAGGACTGGCTTATTCTGACCCCCGGGGTGGGTCTTGACGACAAGGGAGAC | |
| | GCTCTCGGACAGCAGTACCGAACTGTTGAGGATGTCATGTCTACCGGAACGGA | |
| | TATCATAATTGTCGGCCGAGGTCTGTACGGCCAGAACCGAGATCCTATTGAGG | |
| | AGGCCAAGCGATACCAGAAGGCTGGCTGGGAGGCTTACCAGAAGATTAACTGT | |
| | TAGAGGTTAGACTATGGATATGTAATTTAACTGTGTATATAGAGAGCGTGCAA | |
| | GTATGGAGCGCTTGTTCAGCTTGTATGATGGTCAGACGACCTGTCTGATCGAG | |
| | TATGTATGATACTGCACAACCTGTGTATCCGCATGATCTGTCCAATGGGGCAT | |
| | GTTGTTGTGTTTCTCGATACGGAGATGCTGGGTACAAGTAGCTAATACGATTG | |
| | AACTACTTATACTTATATGAGGCTTGAAGAAAGCTGACTTGTGTATGACTTAT | |
| | TCTCAACTACATCCCCAGTCACAATACCACCACTGCACTACCACTACACCTCG | |
| | AGCATGCACACTATTATCACATTACTACATCCAAACCCCTACAGGGGGAGGAG | |
| | CTCTCCAATCAAATATGTACATTAACTATCTCTCGTAAATCATTGTTATAAGA | |
| | CCGTCCGTGACTGTCTAAATCGGTTCATTCGTTGTAACAAATCAGTGTAACAA | |
| | CTCGTGGTACGCCCGGTTTGCTTCGCTCACGCTCCCCTAGATTTTCCGTCTAG | |
| | GACACAACAAGCCCTGGTGGATGACGTTCAACGCCTTCAGCGCGTCTTTTTCC | |
| | TCAATAACACACGAGATGTTAATTTCGTTGGCTCCCTGAGAAATCATCTCAAT | |
| | GTTAATACCTGCCTGGGCCAGAGTAGAGAAGAACATGCCTGCACATCCGACCA | |
| | TGGCCTTCATTCTCGTTCCGATCAGCGACACAATGGTCATGCCTCGCTTCACA | |
| | TCTACTGTGCCGTACTTTCGCAGCTCCTCCACAGCCTGCTTGAGGTTGGAGTC | |
| | GGGAGCATGGAAGGCCATCGACACATGGACCTCAGAAGTGGAGATGAGATCGA | |
| | CGACAAGTTTCTGCTGGTCGAGAGTAGCAAAGATCTTGTTCAAGAAGCCATGA | |
| | CTCTTGGTTCGCTTATTCGAATGAACGTTGAGAACGGTGATATTCGATTTGGT | |
| | AGTGACAGCTGTAGGCTTCTTCTCGTCATCTACAGGAGCAGACACCTGGGGAG | |
| | TGAATGAGCCAGAGGACAGAGAAGTTAGCGAGTCGGTGGAAGAAGCCAGATCC | |
| | TGGACATTGTTTTTCGTTTTCAGGTTGCCCGAGCCGTCAGCAGATGGGTAGAT | |
| | GATAGTTCCTCCTCCCAGGGGGTTTTCCACGTTTTTGATTCGGATAGGAATAT | |
| | GGGCCTTAATGACCTGCTCCATGGTGAAGGGGTGGATGACTTCAGATCCGTAG | |
| | TAGGTCAACTCAGCGGCCTCCTCGGGGGTAATGATGGGCAGCAGACGGGCAGT | |
| | GGACACCTTTCTGGGATCGGCAGTGAAGACACCATCGACTTCTTTCCAGATCT | |
| | GAAGCTCCTTGGCGTCCAGACCCACAGCCACCAGAGCAGCACACAGATCGGTG | |
| | TATCCTCGTCCGATCTGGCTTAGGAGGCCTCCCTTGACGGGGCCAAAGAAACC | |
| | GGTCAACACGGGCACCATGTTTCCCTTGACAGGAGCAGAGTCCGTGTTAGGAG | |
| | AGCCGGGAAGAGTGATGATCTCGCCCAGAACACGTCCGAGGTCCGAATAGAAC | |
| | TTGGGATCTTCGGCATTGGTGGTGGTAACTGCGTGAGACAGGTCAAAGTACCG | |
| | TGCGTTAACACCAGCGTCCCGCATAACTGCAGTCATGTACATGCAACTGAGCT | |
| | TCTCCCCAATGGCCATGATGGAGTCGAGTGTTCGGGGGGAGATCTCAGAAATG | |
| | ATTTCAGCAGCAGCCAGGATTCGCAACAGCTGGTCGCACTCGCCGTTGATGTC | |
| | GGCATTGAGGTTTTCGAGCAGTTCGGGGTTCTTGACGTCACGCTTAGCAGCTG | |
| | CAAGATGGTCTTCTCGGATAGACTCAATGATGGGGTTGTAGGCGTCTGATCCG | |
| | AGAAGAGCAGAGTCAGCAGCAGCAATAAGACGGGTGGTGGTTCCCTCGGCCTT | |
| | GGTGGCCTCTAGATGGCCTCCTTGGCCCCATTCCAGCTGCATTAATGAATCGG | |
| | CCAACGCGCGGGGAGAGGCGGTTTGCGTATTGGGCGCTCTTCCGCTTCCTCGC | |
| | TCACTGACTCGCTGCGCTCGGTCGTTCGGCTGCGGCGAGCGGTATCAGCTCAC | |
| | TCAAAGGCGGTAATACGGTTATCCACAGAATCAGGGGATAACGCAGGAAAGAA | |
| | CATGTGAGCAAAAGGCCAGCAAAAGGCCAGGAACCGTAAAAAGGCCGCGTTGC | |
| | TGGCGTTTTTCCATAGGCTCCGCCCCCCTGACGAGCATCACAAAAATCGACGC | |
| | TCAAGTCAGAGGTGGCGAAACCCGACAGGACTATAAAGATACCAGGCGTTTCC | |
| | CCCTGGAAGCTCCCTCGTGCGCTCTCCTGTTCCGACCCTGCCGCTTACCGGAT | |
| | ACCTGTCCGCCTTTCTCCCTTCGGGAAGCGTGGCGCTTTCTCATAGCTCACGC | |
| | TGTAGGTATCTCAGTTCGGTGTAGGTCGTTCGCTCCAAGCTGGGCTGTGTGCA | |
| | CGAACCCCCCGTTCAGCCCGACCGCTGCGCCTTATCCGGTAACTATCGTCTTG | |
| | AGTCCAACCCGGTAAGACACGACTTATCGCCACTGGCAGCAGCCACTGGTAAC | |
| | AGGATTAGCAGAGCGAGGTATGTAGGCGGTGCTACAGAGTTCTTGAAGTGGTG | |
| | GCCTAACTACGGCTACACTAGAAGAACAGTATTTGGTATCTGCGCTCTGCTGA | |
| | AGCCAGTTACCTTCGGAAAAAGAGTTGGTAGCTCTTGATCCGGCAAACAAACC | |
| | ACCGCTGGTAGCGGTGGTTTTTTTGTTTGCAAGCAGCAGATTACGCGCAGAAA | |
| | AAAAGGATCTCAAGAAGATCCTTTGATCTTTTCTACGGGGTCTGACGCTCAGT | |
| | GGAACGAAAACTCACGTTAAGGGATTTTGGTCATGAGATTATCAAAAAGGATC | |
| | TTCACCTAGATCCTTTTAAATTAAAAATGAAGTTTTAAATCAATCTAAAGTAT | |
| | ATATGAGTAAACTTGGTCTGACAGTTACCAATGCTTAATCAGTGAGGCACCTA | |
| | TCTCAGCGATCTGTCTATTTCGTTCATCCATAGTTGCCTGACTCCCCGTCGTG | |
| | TAGATAACTACGATACGGGAGGGCTTACCATCTGGCCCCAGTGCTGCAATGAT | |
| | ACCGCGAGAGCCACGCTCACCGGCTCCAGATTTATCAGCAATAAACCAGCCAG | |
| | CCGGAAGGGCCGAGCGCAGAAGTGGTCCTGCAACTTTATCCGCCTCCATCCAG | |
| | TCTATTAATTGTTGCCGGGAAGCTAGAGTAAGTAGTTCGCCAGTTAATAGTTT | |
| | GCGCAACGTTGTTGCCATTGCTACAGGCATCGTGGTGTCACGCTCGTCGTTTG | |
| | GTATGGCTTCATTCAGCTCCGGTTCCCAACGATCAAGGCGAGTTACATGATCC | |
| | CCCATGTTGTGCAAAAAAGCGGTTAGCTCCTTCGGTCCTCCGATCGTTGTCAG | |
| | AAGTAAGTTGGCCGCAGTGTTATCACTCATGGTTATGGCAGCACTGCATAATT | |
| | CTCTTACTGTCATGCCATCCGTAAGATGCTTTTCTGTGACTGGTGAGTACTCA | |
| | ACCAAGTCATTCTGAGAATAGTGTATGCGGCGACCGAGTTGCTCTTGCCCGGC | |
| | GTCAATACGGGATAATACCGCGCCACATAGCAGAACTTTAAAAGTGCTCATCA | |
| | TTGGAAAACGTTCTTCGGGGCGAAAACTCTCAAGGATCTTACCGCTGTTGAGA | |
| | TCCAGTTCGATGTAACCCACTCGTGCACCCAACTGATCTTCAGCATCTTTTAC | |
| | TTTCACCAGCGTTTCTGGGTGAGCAAAAACAGGAAGGCAAAATGCCGCAAAAA | |
| | AGGGAATAAGGGCGACACGGAAATGTTGAATACTCATACTCTTCCTTTTTCAA | |
| | TATTATTGAAGCATTTATCAGGGTTATTGTCTCATGAGCGGATACATATTTGA | |
| | ATGTATTTAGAAAAATAAACAAATAGGGGTTCCGCGCACATTTCCCCGAAAAG | |
| | TGCCACCTGACGTCTAAGAAACCATTATTATCATGACATTAACCTATAAAAAT | |
| | AGGCGTATCACGAG | |
| MB8388- | GGTTCCGCTTCCTCGCTCACTGACTCGCTGCGCTCGGTCGTTCGGCTGCGGCG | 9 |
| LIP8 | AGCGGTATCAGCTCACTCAAAGGCGGTAATACGGTTATCCACAGAATCAGGGG | |
| | ATAACGCAGGAAAGAACATGTGAGCAAAAGGCCAGCAAAAGGCCAGGAACCGT | |
| | AAAAAGGCCGCGTTGCTGGCGTTTTTCCATAGGCTCCGCCCCCCTGACGAGCA | |
| | TCACAAAAATCGACGCTCAAGTCAGAGGTGGCGAAACCCGACAGGACTATAAA | |
| | GATACCAGGCGTTTCCCCCTGGAAGCTCCCTCGTGCGCTCTCCTGTTCCGACC | |
| | CTGCCGCTTACCGGATACCTGTCCGCCTTTCTCCCTTCGGGAAGCGTGGCGCT | |
| | TTCTCATAGCTCACGCTGTAGGTATCTCAGTTCGGTGTAGGTCGTTCGCTCCA | |
| | AGCTGGGCTGTGTGCACGAACCCCCCGTTCAGCCCGACCGCTGCGCCTTATCC | |
| | GGTAACTATCGTCTTGAGTCCAACCCGGTAAGACACGACTTATCGCCACTGGC | |
| | AGCAGCCACTGGTAACAGGATTAGCAGAGCGAGGTATGTAGGCGGTGCTACAG | |
| | AGTTCTTGAAGTGGTGGCCTAACTACGGCTACACTAGAAGGACAGTATTTGGT | |
| | ATCTGCGCTCTGCTGAAGCCAGTTACCTTCGGAAAAAGAGTTGGTAGCTCTTG | |
| | ATCCGGCAAACAAACCACCGCTGGTAGCGGTGGTTTTTTTGTTTGCAAGCAGC | |
| | AGATTACGCGCAGAAAAAAAGGATCTCAAGAAGATCCTTTGATCTTTTCTACG | |
| | GGGTCTGACGCTCAGTGGAACGAAAACTCACGTTAAGGGATTTTGGTCATGAG | |
| | ATTATCAAAAAGGATCTTCACCTAGATCCTTTTAAATTAAAAATGAAGTTTTA | |
| | AATCAATCTAAAGTATATATGAGTAAACTTGGTCTGACAGTTACCAATGCTTA | |
| | ATCAGTGAGGCACCTATCTCAGCGATCTGTCTATTTCGTTCATCCATAGTTGC | |
| | CTGACTCCCCGTCGTGTAGATAACTACGATACGGGAGGGCTTACCATCTGGCC | |
| | CCAGTGCTGCAATGATACCGCGAGACCCACGCTCACCGGCTCCAGATTTATCA | |
| | GCAATAAACCAGCCAGCCGGAAGGGCCGAGCGCAGAAGTGGTCCTGCAACTTT | |
| | ATCCGCCTCCATCCAGTCTATTAATTGTTGCCGGGAAGCTAGAGTAAGTAGTT | |
| | CGCCAGTTAATAGTTTGCGCAACGTTGTTGCCATTGCTGCAGGCATCGTGGTG | |
| | TCACGCTCGTCGTTTGGTATGGCTTCATTCAGCTCCGGTTCCCAACGATCAAG | |
| | GCGAGTTACATGATCCCCCATGTTGTGCAAAAAAGCGGTTAGCTCCTTCGGTC | |
| | CTCCGATCGTTGTCAGAAGTAAGTTGGCCGCAGTGTTATCACTCATGGTTATG | |
| | GCAGCACTGCATAATTCTCTTACTGTCATGCCATCCGTAAGATGCTTTTCTGT | |
| | GACTGGTGAGTACTCAACCAAGTCATTCTGAGAATAGTGTATGCGGCGACCGA | |
| | GTTGCTCTTGCCCGGCGTCAACACGGGATAATACCGCGCCACATAGCAGAACT | |
| | TTAAAAGTGCTCATCATTGGAAAACGTTCTTCGGGGCGAAAACTCTCAAGGAT | |
| | CTTACCGCTGTTGAGATCCAGTTCGATGTAACCCACTCGTGCACCCAACTGAT | |
| | CTTCAGCATCTTTTACTTTCACCAGCGTTTCTGGGTGAGCAAAAACAGGAAGG | |
| | CAAAATGCCGCAAAAAAGGGAATAAGGGCGACACGGAAATGTTGAATACTCAT | |
| | ACTCTTCCTTTTTCAATATTATTGAAGCATTTATCAGGGTTATTGTCTCATGA | |
| | GCGGATACATATTTGAATGTATTTAGAAAAATAAACAAATAGGGGTTCCGCGC | |
| | ACATTTCCCCGAAAAGTGCCACCTGACGTCTAAGAAACCATTATTATCATGAC | |
| | ATTAACCTATAAAAATAGGCGTATCACGAGGCCCTTTCGTCTGGCCTAGGAAG | |
| | CGACTTCCAATCGCTTTGCATATCCAGTACCACACCCACAGGCGTTTGTGCTA | |
| | CTCTACTGATAGCAATAGATGCGTCATAATTGGTTGGCCCGCTGAGCCTCCAC | |
| | AGGATACTATTGCACATACCCTGGTCATGTGCAGATCAGCTCATTTGTGGAGA | |
| | CTCTGGAGTAACTTAGACGACGCCTGGTTCAATTGCCGCAATGTGCGCCCACG | |
| | CAGATAATGTATTGAGGGGTGGAGCGCCTCTTGGGGACTTGCTGTACTTGTAC | |
| | GGGATATTAAACGCACTCAGCAAGACCATGACGTAAAACACACCTACTGTACG | |
| | ATACGTACTGTAGGTATTGTACTCGTACCCGGTACTACAAATAGTACGATACT | |
| | ATACGGAGTGTATTTGTACCTTGATATACGACTGGCGGAGTGAAGAGAAGGAG | |
| | TTGAACAAGACCAGATGGGGATATCAGCCCCAGTGCTTTGTATTACAAGTACG | |
| | AGTACTTAATAGATACTGTAAGGCTATTGATACGGATGGCAGTAAGTCATTGA | |
| | GTAAGCAATTGTGGCCCAGCATCTCCCCTACGTACTTGTACCATACCCCATGG | |
| | AGACACCAATGGTCTTTCACGCACACTGTCGTGTGCTGTATCGCAGAATCGGG | |
| | TGTCCAACCAAATGCCGTTACCCCCACGTCACAGCCGATAGACAGATACACCA | |
| | TCAATACCAGCAGGTTGTATCATGCGGTTGGCTGAAGGTAAGCTGATTGGTCT | |
| | AAAAACTGTAGCTGTCCTAATTCAACGAGCGCTATTTGGGGCCAACCACCTCG | |
| | GCCAAGCGGCCTTTAATCTGCGTGCCCCAGAGGCGTCTAATGAGGCTCTGGCC | |
| | GCCACTGTAGGAGTGTTTCTCTGTGCGCACACGCAGTTTTGAGTTTGGGCGAC | |
| | TTTCCCTTTTTCCCAATTGCGTACACACACAGCTCCGAGCTAAGCGCTGTCCT | |
| | TGAACCTTCTCCCTCTTTTCCCTCTTTTTCTCTTCCCCTTCCCCTCCTCCACA | |
| | TTAAGGCCAAATCCTGAATTGCACCAACTAGTACAACGACAACAATGGACAAG | |
| | AAGTACTCCATCGGTTTGGACATTGGTACTAACTCTGTCGGCTGGGCCGTCAT | |
| | CACCGACGAGTACAAGGTTCCCTCCAAGAAGTTCAAGGTCCTTGGCAACACCG | |
| | ACCGACACTCTATCAAGAAGAACCTGATCGGTGCTCTGCTGTTCGACTCTGGC | |
| | GAGACTGCCGAGGCCACCCGACTGAAGCGAACCGCTCGACGCCGATACACCCG | |
| | ACGAAAGAACCGAATCTGTTACCTCCAGGAGATCTTCAGCAACGAGATGGCTA | |
| | AGGTCGACGACTCCTTCTTCCACCGACTCGAGGAGTCTTTCCTGGTCGAAGAG | |
| | GATAAGAAGCACGAGCGACACCCCATCTTCGGCAACATTGTTGATGAGGTTGC | |
| | CTACCATGAGAAGTACCCCACCATCTACCACCTCCGAAAGAAGCTCGTCGACT | |
| | CCACTGACAAGGCTGACCTCCGACTCATCTACCTTGCTCTCGCCCACATGATC | |
| | AAGTTCCGAGGTCACTTCCTCATTGAGGGTGATCTCAACCCCGACAACTCCGA | |
| | CGTTGACAAGCTGTTCATCCAGCTCGTCCAGACCTACAACCAGCTCTTTGAGG | |
| | AGAACCCTATCAACGCTTCTGGTGTTGACGCCAAGGCCATTCTCTCCGCCCGA | |
| | CTCTCTAAGTCCCGACGACTCGAGAACCTCATTGCCCAGCTGCCCGGCGAGAA | |
| | GAAGAACGGCCTCTTCGGTAACCTGATTGCTCTCTCTCTTGGTCTGACCCCCA | |
| | ACTTCAAGTCCAACTTTGACCTCGCCGAGGACGCCAAGCTCCAGCTGTCCAAG | |
| | GACACCTACGATGACGATCTGGACAACCTCCTGGCCCAGATCGGTGACCAGTA | |
| | CGCCGATCTCTTCCTTGCCGCCAAGAACCTCTCCGACGCCATCCTGCTCTCCG | |
| | ACATCCTCCGAGTCAACACCGAGATTACCAAGGCTCCTCTGTCTGCCTCTATG | |
| | ATCAAGCGATACGACGAGCACCACCAGGATCTCACTCTTCTCAAGGCTCTCGT | |
| | CCGACAGCAGCTCCCCGAGAAGTACAAGGAGATTTTCTTTGACCAGTCCAAGA | |
| | ACGGTTACGCTGGCTACATTGACGGTGGTGCTTCCCAGGAAGAGTTTTACAAG | |
| | TTCATCAAGCCTATTCTGGAGAAGATGGACGGTACCGAGGAGCTGCTCGTCAA | |
| | GCTCAACCGAGAGGACCTCCTTCGAAAGCAGCGAACCTTCGATAACGGCTCCA | |
| | TCCCCCACCAGATCCACCTGGGTGAGCTCCACGCCATTCTCCGAAGACAAGAG | |
| | GACTTCTACCCCTTCCTAAAGGATAACCGAGAGAAGATCGAGAAGATTCTCAC | |
| | CTTCCGAATCCCCTACTACGTCGGTCCCCTCGCTCGAGGTAACTCCCGATTTG | |
| | CTTGGATGACCCGAAAGTCCGAGGAGACTATCACCCCCTGGAACTTTGAAGAG | |
| | GTAGTCGACAAGGGTGCCTCCGCCCAGTCTTTCATTGAGCGGATGACCAACTT | |
| | CGATAAGAACCTCCCCAACGAGAAGGTCCTTCCCAAGCACTCTCTCCTCTACG | |
| | AGTACTTCACCGTCTACAACGAGCTGACCAAGGTCAAGTACGTTACCGAGGGC | |
| | ATGCGAAAGCCCGCTTTCCTCTCTGGTGAGCAGAAGAAGGCCATTGTCGACCT | |
| | CCTGTTCAAGACTAACCGAAAAGTCACCGTCAAGCAGCTCAAGGAAGACTACT | |
| | TCAAGAAGATTGAGTGCTTCGACTCCGTCGAGATTTCCGGTGTCGAGGACCGA | |
| | TTCAACGCCTCCCTCGGCACCTACCACGATCTTCTGAAGATCATCAAGGACAA | |
| | GGACTTTCTTGATAACGAGGAGAACGAGGACATTCTCGAGGACATCGTCCTCA | |
| | CCCTCACCCTTTTCGAGGATCGAGAGATGATCGAGGAGCGACTCAAGACCTAC | |
| | GCCCATCTCTTCGACGACAAGGTCATGAAGCAACTCAAGCGACGACGATACAC | |
| | TGGCTGGGGCCGACTTTCCCGAAAGCTCATCAACGGCATCCGAGACAAGCAGT | |
| | CTGGCAAGACCATCCTGGACTTCCTGAAGTCCGACGGTTTCGCCAACCGAAAC | |
| | TTCATGCAGCTCATCCACGACGACTCTCTTACCTTCAAAGAGGATATCCAGAA | |
| | GGCCCAGGTTTCTGGCCAGGGCGACTCCCTCCACGAGCACATTGCCAACCTCG | |
| | CCGGATCCCCCGCCATCAAAAAGGGTATCCTCCAGACCGTCAAGGTTGTCGAC | |
| | GAACTCGTGAAGGTCATGGGCCGACACAAGCCCGAGAACATCGTTATCGAGAT | |
| | GGCCCGAGAGAACCAGACCACCCAGAAGGGTCAGAAGAACTCCCGAGAGCGAA | |
| | TGAAGCGAATCGAAGAGGGTATCAAGGAGCTCGGTTCCCAGATTCTCAAGGAG | |
| | CACCCCGTCGAGAACACCCAGCTCCAGAACGAGAAACTCTACCTGTACTACCT | |
| | CCAGAATGGCCGAGACATGTACGTTGACCAGGAGCTCGACATCAACCGACTCT | |
| | CCGACTACGACGTCGACCACATTGTTCCTCAGTCCTTCCTCAAGGACGACTCC | |
| | ATCGACAACAAGGTTCTGACCCGATCTGACAAGAACCGAGGTAAGTCCGACAA | |
| | CGTTCCCTCCGAAGAGGTCGTTAAGAAGATGAAGAACTACTGGCGACAGCTTC | |
| | TCAACGCCAAACTGATCACCCAGCGAAAGTTTGACAACCTCACCAAGGCCGAG | |
| | CGAGGTGGTCTGTCCGAGCTGGACAAGGCCGGCTTCATTAAGCGACAGCTGGT | |
| | CGAGACTCGACAGATCACCAAGCACGTCGCCCAGATCCTCGACTCCCGAATGA | |
| | ACACCAAGTACGACGAGAACGACAAGCTCATCCGGGAGGTCAAGGTCATCACC | |
| | CTGAAGTCTAAGCTTGTCTCCGACTTCCGAAAGGACTTCCAGTTCTACAAGGT | |
| | CCGAGAGATCAACAACTACCACCACGCCCACGACGCCTACCTCAACGCCGTTG | |
| | TTGGTACCGCCCTCATCAAGAAGTATCCCAAGCTCGAGTCCGAGTTCGTTTAC | |
| | GGCGACTACAAGGTTTACGATGTCCGAAAGATGATTGCCAAGTCCGAGCAGGA | |
| | GATCGGTAAGGCCACCGCCAAGTACTTTTTCTACTCCAACATCATGAATTTCT | |
| | TCAAGACCGAGATCACTCTCGCCAACGGTGAGATTCGAAAGCGACCCCTGATT | |
| | GAGACTAATGGTGAGACTGGTGAGATCGTCTGGGATAAGGGCCGAGACTTCGC | |
| | CACCGTCCGAAAGGTCCTGTCCATGCCCCAGGTCAACATTGTCAAGAAGACCG | |
| | AGGTCCAGACCGGTGGCTTCTCCAAGGAGTCCATTCTCCCCAAGCGAAACTCC | |
| | GACAAACTCATCGCCCGTAAGAAGGACTGGGATCCGAAGAAGTACGGTGGTTT | |
| | CGATTCTCCCACCGTTGCCTACTCCGTCCTCGTTGTTGCTAAAGTCGAGAAGG | |
| | GTAAGTCTAAGAAACTCAAGTCCGTGAAGGAGCTACTCGGTATCACCATCATG | |
| | GAGCGATCTTCTTTTGAGAAGAACCCCATTGACTTCCTCGAGGCCAAGGGTTA | |
| | CAAAGAGGTCAAGAAGGACCTGATTATCAAGCTGCCCAAGTACTCCCTCTTTG | |
| | AGCTCGAGAACGGCCGAAAGCGAATGCTGGCTTCCGCTGGTGAGCTGCAGAAG | |
| | GGCAACGAGCTCGCTCTGCCCTCCAAGTACGTCAACTTCCTCTACCTGGCCTC | |
| | CCACTACGAGAAGCTCAAGGGCTCCCCCGAGGACAACGAGCAGAAGCAGCTGT | |
| | TCGTTGAGCAGCACAAGCACTACCTCGACGAGATCATCGAGCAGATCTCCGAG | |
| | TTCTCCAAGCGAGTCATCCTCGCTGACGCCAACCTTGATAAGGTTCTCTCTGC | |
| | TTACAACAAGCACCGGGACAAGCCCATCCGAGAGCAGGCCGAGAATATCATCC | |
| | ACCTCTTCACTCTCACCAACCTCGGCGCTCCTGCTGCCTTCAAGTACTTCGAC | |
| | ACCACCATTGACCGAAAGAGGTACACCTCCACCAAGGAAGTCCTCGACGCCAC | |
| | CCTGATCCACCAGTCCATCACCGGCCTCTACGAAACCCGAATCGACCTCTCCC | |
| | AGCTCGGCGGTGACTCTCGAGCCGACCCCAAGAAGAAGCGAAAAGTCTAAATA | |
| | TCCGAAGATCAAGAGCGAAGCAAGTTGTAAGTCCAGGACATGTTTCCCGCCCA | |
| | CGCGAGTGATTTATAACACCTCTCTTTTTTGACACCCGCTCGCCTTGAAATTC | |
| | ATGTCACATAAATTATAGTCAACGACGTTTGAATAACTTGTCTTGTAGTTCGA | |
| | TGATGATCATATGATTACATTAATAGTAATTACTGTATTTGATATATATACTA | |
| | ATTACAATAGTACATATTAGAACATACAATAGTTAGTGCCGTGAAGTGGCTTA | |
| | AAATACCGCGAGTCGATTACGTAATATTATTACCTCTTGCCCATCGAACGTAC | |
| | AAGTACTCCTCTGTTCTCTCCTTCCTTTGCTTTGTGCACGAAGAACTGCGGTC | |
| | AGGTGACACAACTTTTTCCATCTCAGGGTGTGTCGCGTGTGCTTCATCCAAAC | |
| | TTTAGTTGGGGTTCGGGTTCGCGCGAGATGATCACGTGCCCTGATTTGGTGTC | |
| | GTCCCCCGTCGCGCTGCGCACGTGATTTATTTATTTCCGGTGGCTGCTGTCTA | |
| | CGCGGGGCCTTCTCTGCCCTTCTGTTTCAACCTTCGGGCGGTTCTCGTAACCA | |
| | GCAGTAGCAATCCATTTCGAAACTCAAAGAGCTAAAAACGTTAAACCTCAGCA | |
| | GTCGCTCGACGAATGGGCTGCGGTTGGGAAGCCCACGAGGCCTATAGCCAGAG | |
| | CCTCGAGTTGACAGGAGCCCAGACGCCTTTTCCAACGGCAACTTTTATATAAA | |
| | ATGGCAATGTATTCATGCAATTGCGGCCGTGTCAGGTTGGAGACACTGGACCA | |
| | CACTCTCCATTGCTTCCTGAGGAGATGGATCATTGCTAGTGCATCTACGCGCA | |
| | GCAATCCCGCAAGCTCGACAACCGTAGATGGGCTTTGGTGGGCCAATCAATTA | |
| | CGCAACCCGCACGTTAAATTGTATGAGGAAGGAAGGCCACGGTACAAAGTGGG | |
| | TGGTCTTCACCCAGTGGTTGTTGGTGGCGTCATGCAGACCATGCATTGGGGAT | |
| | AGCACAGGGTTGGGGTGTCTTGTGGACTCAATGGGTGAAAGGAGATGGAAAAG | |
| | GGCGGTGAAAAGTGGTAGAATCGAAATCCCTGACGTCAATTTATAAAGTAAAA | |
| | TGCGTTTCTGCCATTTTGCTCCCCTCCTTCTTTCGCAATCGCCTCCCCAAAAG | |
| | TTGTCGTGGCAGTACACATGCTTGCATACAATGAAGCTAATCCGGCTTGCTCA | |
| | GTAGTTGCTATATCCAGGCATGGTGTGAAACCCCTCAAAGTATATATAGGAGC | |
| | GGTGAGCCCCAGTCTGGGGTCTTTTCTCTCCATCTCAAAACTACTTTCTCACA | |
| | ATGACGAGACTGATGAGTCCGTGAGGACGAAACGAGTAAGCTCGTCGAGGGCT | |
| | GCAAAATTCACGAGTTTTAGAGCTAGAAATAGCAAGTTAAAATAAGGCTAGTC | |
| | CGTTATCAACTTGAAAAAGTGGCACCGAGTCGGTGCTTTTGGCCGGCATGGTC | |
| | CCAGCCTCCTCGCTGGCGCCGGCTGGGCAACATGCTTCGGCATGGCGAATGGG | |
| | ACTAAACTTCGAGCTAATCCAGTAGCTTACGTTACCCAGGGGCAGGTCAACTG | |
| | GCTAGCCACGAGTCTGTCCCAGGTCGCAATTTAGTGTAATAAACAATATATAT | |
| | ATTGAGTCTAAAGGGAATTGTAGCTATTGTGATTGTGTGATTTTCGTCTTGCT | |
| | GGTTCTTATTGTGTCCCATTCGTTTCATCCTGATGAGGACCCCTGGAACCGGT | |
| | GTTTTCTTAGTCTCTGCAATCGCTAGTCTTGTTGCTATGACAGTTGCGTCGAC | |
| | ACTATTCAGGTCATCTATCGGTTATTCTGATATTATAATACCTCCGGATCGAT | |
| | GTACCTGATTTATACTTGCAGCAATGTTTACTTCTTATCGTTGGACCCCGTCT | |
| | TCAATTACACTTCCCAACTGGGAACACCCCTCTTTATCGACCCATTTTAGGTA | |
| | ATTTACCCTAGCCCATTGTCTCCATAAGGAATATTACCCTAACCCACAGTCCA | |
| | GGGTGCCCAGGTCCTTCTTTGGCCAAATTTTAACTTCGGTCCTATGGCACAGC | |
| | GGTAGCGCGTGAGATTGCAAATCTTAAGGTCCCGAGTTCGAATCTCGGTGGGA | |
| | CCTAGTTGAAAAATACCTCTAATGCGCCGATGGTTTAGTGGTAAAATCCATCG | |
| | TTGCCATCGATGGGCCCCCGGTTCGATTCCGGGTCGGCGCAGGTTGACGTACA | |
| | GCAGGCTGAACGAGGATGTTTTAGAGCTAGAAATAGCAAGTTAAAATAAGGCT | |
| | AGTCCGTTATCAACTTGAAAAAGTGGCACCGAGTCGGTGCTTTTTTTTTTCGC | |
| | GATACACGAATGTGATACGGATCAAAGTAAGCAGGACTACGATAAGATAACGA | |
| | ATGCGGTGCAGTCCATGTCGATTAGGTATAGATACATTTATTTTGTGTTATGT | |
| | TACATTTTGGGGGGATACTGTCCTACTTGTAGTACCTACTTGTAGTGGCGCGT | |
| | CTATTCCTTTGCCCTCGGACGAGTGCTGGGGCGTCGGTTTCCACTATCGGCGA | |
| | GTACTTCTACACAGCCATCGGTCCAGACGGCCGCGCTTCTGCGGGCGATTTGT | |
| | GTACGCCCGACAGTCCCGGCTCCGGATCGGACGATTGCGTCGCATCGACCCTG | |
| | CGCCCAAGCTGCATCATCGAAATTGCCGTCAACCAAGCTCTGATAGAGTTGGT | |
| | CAAGACCAATGCGGAGCATATACGCCCGGAGCCGCGGCGATCCTGCAAGCTCC | |
| | GGATGCCTCCGCTCGAAGTAGCGCGTCTGCTGCTCCATACAAGCCAACCACGG | |
| | CCTCCAGAAGAAGATGTTGGCGACCTCGTATTGGGAATCCCCGAACATCGCCT | |
| | CGCTCCAGTCAATGACCGCTGTTATGCGGCCATTGTCCGTCAGGACATTGTTG | |
| | GAGCCGAAATCCGCGTGCACGAGGTGCCGGACTTCGGGGCAGTCCTCGGCCCA | |
| | AAGCATCAGCTCATCGAGAGCCTGCGCGACGGACGCACTGACGGTGTCGTCCA | |
| | TCACAGTTTGCCAGTGATACACATGGGGATCAGCAATCGCGCATATGAAATCA | |
| | CGCCATGTAGTGTATTGACCGATTCCTTGCGGTCCGAATGGGCCGAACCCGCT | |
| | CGTCTGGCTAAGATCGGCCGCAGCGATCGCATCCATGGCCTCCGCGACCGGCT | |
| | GCAGAACAGCGGGCAGTTCGGTTTCAGGCAGGTCTTGCAACGTGACACCCTGT | |
| | GCACGGCGGGAGATGCAATAGGTCAGGCTCTCGCTGAATTCCCCAATGTCAAG | |
| | CACTTCCGGAATCGGGAGCGCGGCCGATGCAAAGTGCCGATAAACATAACGAT | |
| | CTTTGTAGAAACCATCGGCGCAGCTATTTACCCGCAGGACATATCCACGCCCT | |
| | CCTACATCGAAGCTGAAAGCACGAGATTCTTCGCCCTCCGAGAGCTGCATCAG | |
| | GTCGGAGACGCTGTCGAACTTTTCGATCAGAAACTTCTCGACAGACGTCGCGG | |
| | TGAGTTCAGGCTTTTTCATATGGGTACCTGAGAACATTTTTGTGTCTAGGTGT | |
| | TTGTGTTTGGACTGCGATCAGTGAAGAAAAGAAGAGGAAAAATTGTGCAAGAA | |
| | ATTTTGCTTTCAAGACTTGGCTGATGCAGCAGGGTAACTCTGGGACACAGACC | |
| | TATGTTTGTGGTTAAACTCAATGCACGTGGTACGTGCGTGGAGCGCTTACCCA | |
| | TCCAAGGGTGTGGACATGGAACCGACGGTCCGTGGAGTTGTGTAATGTCATTT | |
| | TGGCGACTCTTGAAGCAAGGCTATAAAAAAATTGTGTGGCTTGAGTCTTATCG | |
| | AGCTCGGTCACTACAAGAGTTAATCTTCCTGTCTCAGGCAGACAGGTCAGGCA | |
| | GGGTTACTTTTGGGTGTGCTGTAACTCACTGTATGGCCGTTAGTGCGCATAGA | |
| | CGTTGTACATACTGGACCGAATTGTAGCGTGCTCAATAGGGCCAATAAAGCTA | |
| | TTGTAGGGATCCGAATTTTCAGAACCTAATTTATCTGTTACCCGGCCTGTGGC | |
| | TCGCACAGCTTAAAAATGGTCAAACTTTCCCCTTCTTGTCTTTTTTTCCTCAC | |
| | ATTCATCAGGTTCTTGTCTTGATCTTTCAAGTGAGTATTAATTACCGACCTTG | |
| | GTTCTTCATTGGGAGAGCATTGGAAGCCGTGGTGCAGCAACCACAAAACGGTT | |
| | CTTCCCCTTCGATACCTTCTTGCCTGCCTTTCAATACAAGTCGGCTCGATTAG | |
| | CGGTGGTCGCCCCCGCCAGCGGAGAACATGGAACTAACCCAGAATGAGAGCTA | |
| | AGTGGAGAAAGAAGAGAGTCAGACGACTCAAGCGAAAGCGCCGCAAGGTCCGA | |
| | GCTCGATCCAAATAAGCGGTTTTTAACGGAGATTTAACACTAAATCGAAGAAC | |
| | TTTTCCCGTTTCATTTGCGAATGAGCTCGTTAACAAAATCCCCCAGTTTTTTT | |
| | ATCCAGCTGTAAGGATTGACATTAGTAATGAATTATTGTTTGGTATATTTAAA | |
| | TCTGTAGTTCCTTTCTGTCCGTGTCGGCAACTGTCGTACTCGTGATTTACTTG | |
| | TATTGACGAATACTTACTGTAGCGCACTCTGCTGCTACTGGTCGTAAGGATGT | |
| | GCTATTTCGGTGTATGGTGGGTTTTTTGGGGGTCGGAACCGAAGACTGTTACA | |
| | CGGGCACGGCTCGTTGTGTACACGCACAGAGCTCTTGCGAGTCATGTTGTAGC | |
| | TAGCTCGTCGTGTTCAGGAACTGTTCGATGGTTCGGAGAGAGTCGCCGCCCAG | |
| | AACATACGCGCACCGATGTCAGCAGACAGCCTTATTACAAGTATATTCAAGCA | |
| | AGTATATCCGTAGGGTGCGGGTGATTTGGATCTAAGGTTCGTACTCAACACTC | |
| | ACGAGCAGCTTGCCTATGTTACATCCTTTTATCAGACATAACATAATTGGAGT | |
| | TTACTTACACACGGGGTGTACCTGTATGAGCACCACCTACAATTGTAGCACTG | |
| | GTACTTGTACAAAGAATTTATTCGTACGAATCACAGGGACGGCCGCCCTCACC | |
| | GAACCAGCGAATACCTCAGCGGTCCCCTGCAGTGACTCAACAAAGCGATATGA | |
| | ACATCTTGCGATGGTATCCTGCTGATAGTTTTTACTGTACAAACACCTGTGTA | |
| | GCTCCTTCTAGCATTTTTAAGTTATTCACACCTCAAGGGGAGGGATAAATTAA | |
| | ATAAATTCCAAAAGCGAAGATCGAGAAACTAAATTAAAATTCCAAAAACGAAG | |
| | TTGGAACACAACCCCCCGAAAAAAAACAACAAACAAAAAACCCAACAAAATAA | |
| | ACAAAAACAAAATAAATATATAACTACCAGTATCTGACTAAAAGTTCAAATAC | |
| | TCGTACTTACAACAAATAGAAATGAGCCGGCCAAAATTCTGCAGAAAAAAATT | |
| | TCAAACAAGTACTGGTATAATTAAATTAAAAAACACATCAAAGTATCATAACG | |
| | TTAGTTATTTTATTTTATTTAATAAAAGAAAACAACAAGATGGGCTCAAAACT | |
| | TTCAACTTATACGATACATACCAAATAACAATTTAGTATTTATCTAAGTGCTT | |
| | TTCGTAGATAATGGAATACAAATGGATATCCAGAGTATACACATGGATAGTAT | |
| | ACACTGACACGACAATTCTGTATCTCTTTATGTTAACTACTGTGAGGCATTAA | |
| | ATAGAGCTTGATATATAAAATGTTACATTTCACAGTCTGAACTTTTGCAGATT | |
| | ACCTAATTTGGTAAGATATTAATTATGAACTGAAAGTTGATGGCATCCCTAAA | |
| | TTTGATGAAAGATGAAATTGTAAATGAGGTGGTAAAAGAGCTACAGTCGTTTT | |
| | GTTTTGAGATACCATCATCTCTAACGAAATATCTATTAAAAATCTCAGTGTGA | |
| | TCATGAGTCATTGCCATCCTGGAAAATGTCATCATGGCTGATATTTCTAACTG | |
| | TTTACTTGAGATAAATATATATTTACAAGAACTTCCCTTGAAATTAATTTAGA | |
| | TATAAAATGTTTGCGGGCAAGTTACTACGAGGAATAAATTATATCTAGAAAGC | |
| | GGAAGAGCGCCCAATACGCAAACCGCCTCTCCCCGCGCGTTGGCCGATTCATT | |
| | AATGCAGCTGGCACGACAGGTTTCCCGACTGGAAAGCGGGCAGTGAGCGCAAC | |
| | GCAATTAATGTGAGTTAGCTCACTCATTAGGCACCCCAGGCTTTACACTTTAT | |
| | GCTTCCGGCTCGTATGTTGTGTGGAATTGTGAGCGGATAACAATTTCACACAG | |
| | GAAACAGCTATGACCATGATTACGCCAAGCTTGGTACCAGAGACGGGTTGGCG | |
| | GCGTATTTGTGTCCCAAAAAACAGCCCCAATTGCCCCAATTGACCCCAAATTG | |
| | ACCCAGTAGCGGGCCCAACCCCGGCGAGAGCCCCCTTCACCCCACATATCAAA | |
| | CCTCCCCCGGTTCCCACACTTGCCGTTAAGGGCGTAGGGTACTGCAGTCTGGA | |
| | ATCTACGCTTGTTCAGACTTTGTACTAGTTTCTTTGTCTGGCCATCCGGGTAA | |
| | CCCATGCCGGACGCAAAATAGACTACTGAAAATTTTTTTGCTTTGTGGTTGGG | |
| | ACTTTAGCCAAGGGTATAAAAGACCACCGTCCCCGAATTACCTTTCCTCTTCT | |
| | TTTCTCTCTCTCCTTGTCAACTCACACCCGAAATCGTTAAGCATTTCCTTCTG | |
| | AGTATAAGAATCATTCGCTAGCCACAAAAATGATGGCTCCCTCCATTCGAAAG | |
| | TTCTTTGCTGGTGGTGTGTGTCGAACTAACGTTCAGCTTCCCGGTAAGGTGGT | |
| | TGTCATCACTGGTGCCAACACCGGCATTGGCAAGGAGACTGCCCGAGAGCTCG | |
| | CTTCCCGAGGAGCCCGAGTTTACATTGCTTGCCGAGATGTTCTGAAGGGCGAG | |
| | TCTGCTGCCTCTGAGATTCGAGTTGACACTAAGAACTCCCAGGTGCTCGTGCG | |
| | AAAGCTCGACCTTTCCGACACTAAGTCTATCCGAGCCTTTGCTGAGGGCTTTC | |
| | TCGCTGAGGAGAAGCAGCTTCACATTCTGATTAACAACGCTGGAGTTATGATG | |
| | TGTCCTTACTCTAAGACTGCTGATGGTTTCGAGACTCACCTCGGAGTCAACCA | |
| | CCTGGGCCACTTCCTCCTCACCTACCTGCTCCTGGAGCGACTCAAGGTGTCTG | |
| | CCCCTGCCCGAGTGGTTAACGTTTCCTCCGTGGCTCACCACATTGGCAAGATT | |
| | CCCTTCCACGACCTCCAGTCCGAGAAGCGATACTCCCGAGGTTTTGCTTACTG | |
| | CCACTCCAAGCTGGCCAACGTTCTCTTTACCCGAGAGCTGGCCAAGCGACTCC | |
| | AGGGAACCGGCGTCACCACCTACGCCGTTCACCCCGGTGTCGTCCGATCCGAG | |
| | CTGGTCCGACACTCCTCCCTGCTCTGCCTGCTCTGGCGACTCTTCTCCCCCTT | |
| | CGTTAAGACCGCCCGAGAGGGTGCCCAGACCTCCCTGCACTGCGCCCTGGCTG | |
| | AGGGCCTGGAGCCCCTGTCTGGCAAGTACTTCTCTGACTGCAAGCGAACCTGG | |
| | GTGTCTCCCCGAGCTCGAAACAACAAGACTGCCGAGCGACTCTGGAACGTTTC | |
| | CTGTGAGCTTCTCGGTATTCGATGGGAGTAGACGCGTGCAATTAACAGATAGT | |
| | TTGCCGGTGATAATTCTCTTAACCTCCCACACTCCTTTGACATAACGATTTAT | |
| | GTAACGAAACTGAAATTTGACCAGATATTGTTGTAAATAGAAAATCTGGCTTG | |
| | TAGGTGGCAAACTAGTGGTGTGTTCTGTGGAGCATTCTCACTTTTGGTAAACG | |
| | ACATTGCTTCAAGTGCAGCGGAATCAAAAAGTATAAAGTGGGCAGCGAGTATA | |
| | CCTGTACAGACTGTAGGCGATAACTCAATCCAATTACCCCCCACAACATGACT | |
| | GGCCAAACTGATCTCAAGACTTTATTGAAATCAGCAACACCGATTCTCAATGA | |
| | AGGCACATACTTCTTCTGCAACATTCACTTGACGCCTAAAGTTGGTGAGAAAT | |
| | GGACCGACAAGACATATTCTGCTATCCACGGACTGTTGCCTGTGTCGGTGGCT | |
| | ACAATACGTGAGTCAGAAGGGCTGACGGTGGTGGTTCCCAAGGAAAAGGTCGA | |
| | CGAGTATCTGTCTGACTCGTCATTGCCGCCTTTGGAGTACGACTCCAACTATG | |
| | AGTGTGCTTGGATCACTTTGACGATACATTCTTCGTTGGAGGCTGTGGGTCTG | |
| | ACAGCTGCGTTTTCGGCGCGGTTGGCCGACAACAATATCAGCTGCAACGTCAT | |
| | TGCTGGCTTTCATCATGATCACATTTTTGTCGGCAAAGGCGACGCCCAGAGAG | |
| | CCATTGACGTTCTTTCTAATTTGGACCGATAGCCGTATAGTCCAGTCTATCTA | |
| | TAAGTTCAACTAACTCGTAACTATTACCATAACATATACTTCACTGCCCCAGA | |
| | TAAGGTTCCGATAAAAAGTTCTGCAGACTAAATTTATTTCAGTCTCCTCTTCA | |
| | CCACCAAAATGCCCTCCTACGAAGCTCGAGCTAACGTCCACAAGTCCGCCTTT | |
| | GCCGCTCGAGTGCTCAAGCTCGTGGCAGCCAAGAAAACCAACCTGTGTGCTTC | |
| | TCTGGATGTTACCACCACCAAGGAGCTCATTGAGCTTGCCGATAAGGTCGGAC | |
| | CTTATGTGTGCATGATCAAGACCCATATCGACATCATTGACGACTTCACCTAC | |
| | GCCGGCACTGTGCTCCCCCTCAAGGAACTTGCTCTTAAGCACGGTTTCTTCCT | |
| | GTTCGAGGACAGAAAGTTCGCAGATATTGGCAACACTGTCAAGCACCAGTACA | |
| | AGAACGGTGTCTACCGAATCGCCGAGTGGTCCGATATCACCAACGCCCACGGT | |
| | GTACCCGGAACCGGAATCATTGCTGGCCTGCGAGCTGGTGCCGAGGAAACTGT | |
| | CTCTGAACAGAAGAAGGAGGACGTCTCTGACTACGAGAACTCCCAGTACAAGG | |
| | AGTTCCTGGTCCCCTCTCCCAACGAGAAGCTGGCCAGAGGTCTGCTCATGCTG | |
| | GCCGAGCTGTCTTGCAAGGGCTCTCTGGCCACTGGCGAGTACTCCAAGCAGAC | |
| | CATTGAGCTTGCCCGATCCGACCCCGAGTTTGTGGTTGGCTTCATTGCCCAGA | |
| | ACCGACCTAAGGGCGACTCTGAGGACTGGCTTATTCTGACCCCCGGGGTGGGT | |
| | CTTGACGACAAGGGAGACGCTCTCGGACAGCAGTACCGAACTGTTGAGGATGT | |
| | CATGTCTACCGGAACGGATATCATAATTGTCGGCCGAGGTCTGTACGGCCAGA | |
| | ACCGAGATCCTATTGAGGAGGCCAAGCGATACCAGAAGGCTGGCTGGGAGGCT | |
| | TACCAGAAGATTAACTGTTAGAGGTTAGACTATGGATATGTAATTTAACTGTG | |
| | TATATAGAGAGCGTGCAAGTATGGAGCGCTTGTTCAGCTTGTATGATGGTCAG | |
| | ACGACCTGTCTGATCGAGTATGTATGATACTGCACAACCTGTGTATCCGCATG | |
| | ATCTGTCCAATGGGGCATGTTGTTGTGTTTCTCGATACGGAGATGCTGGGTAC | |
| | AAGTAGCTAATACGATTGAACTACTTATACTTATATGAGGCTTGAAGAAAGCT | |
| | GACTTGTGTATGACTTATTCTCAACTACATCCCCAGTCACAATACCACCACTG | |
| | CACTACCACTACACCTCGAGCATGCATCTAGTGGTGTGTTCTGTGGAGCATTC | |
| | TCACTTTTGGTAAACGACATTGCTTCAAGTGCAGCGGAATCAAAAAGTATAAA | |
| | GTGGGCAGCGAGTATACCTGTACAGACTGTAGGCGATAACTCAATCCAATTAC | |
| | CCCCCACAACATGACTGGCCAAACTGATCTCAAGACTTTATTGAAATCAGCAA | |
| | CACCGATTCTCAATGAAGGCACATACTTCTTCTGCAACATTCACTTGACGCCT | |
| | AAAGTTGGTGAGAAATGGACCGACAAGACATATTCTGCTATCCACGGACTGTT | |
| | GCCTGTGTCGGTGGCTACAATACGTGAGTCAGAAGGGCTGACGGTGGTGGTTC | |
| | CCAAGGAAAAGGTCGACGAGTATCTGTCTGACTCGTCATTGCCGCCTTTGGAG | |
| | TACGACTCCAACTATGAGTGTGCTTGGATCACTTTGACGATACATTCTTCGTT | |
| | GGAGGCTGTGGGTCTGACAGCTGCGTTTTCGGCGCGGTTGGCCGACAACAATA | |
| | TCAGCTGCAACGTCATTGCTGGCTTTCATCATGATCACATTTTTGTCGGCAAA | |
| | GGCGACGCCCAGAGAGCCATTGACGTTCTTTCTAATTTGGACCGATAGCCGTA | |
| | TAGTCCAGTCTATCTATAAGTTCAACTAACTCGTAACTATTACCATAACATAT | |
| | ACTTCACTGCCCCAGATAAGGTTCCGATAAAAAGTTCTGCAGACTAAATTTAT | |
| | TTCAGTCTCCTCTTCACCACCAAAATGCCCTCCTACGAAGCTCGAGCTAACGT | |
| | CCACAAGTCCGCCTTTGCCGCTCGAGTGCTCAAGCTCGTGGCAGCCAAGAAAA | |
| | CCAACCTGTGTGCTTCTCTGGATGTTACCACCACCAAGGGGCCGCTCGAGCAT | |
| | GCATCTAGAGGGCCCAATTCGCCCTATAGTGAGTCGTATTACAATTCACTGGC | |
| | CGTCGTTTTACAACGTCGTGACTGGGAAAACCCTGGCGTTACCCAACTTAATC | |
| | GCCTTGCAGCACATCCCCCTTTCGCCAGCTGGCGTAATAGCGAAGAGGCCCGC | |
| | ACCGATCGCCCTTCCCAACAGTTGCGCAGCCTGAATGGCGAATGGACGCGCCC | |
| | TGTAGCGGCGCATTAAGCGCGGCGGGTGTGGTGGTTACGCGCAGCGTGACCGC | |
| | TACACTTGCCAGCGCCCTAGCGCCCGCTCCTTTCGCTTTCTTCCCTTCCTTTC | |
| | TCGCCACGTTCGCCGGCTTTCCCCGTCAAGCTCTAAATCGGGGGCTCCCTTTA | |
| | GGGTTCCGATTTAGTGCTTTACGGCACCTCGACCCCAAAAAACTTGATTAGGG | |
| | TGATGGTTCACGTAGTGGGCCATCGCCCTGATAGACGGTTTTTCGCCCTTTGA | |
| | CGTTGGAGTCCACGTTCTTTAATAGTGGACTCTTGTTCCAAACTGGAACAACA | |
| | CTCAACCCTATCTCGGTCTATTCTTTTGATTTATAAGGGATTTTGCCGATTTC | |
| | GGCCTATTGGTTAAAAAATGAGCTGATTTAACAAAAATTTAACGCGAATTTTA | |
| | ACAAAATTCAGGGCGCAAGGGCTGCTAAAGGAAGCGGAACACGTAGAAAGCCA | |
| | GTCCGCAGAAACGGTGCTGACCCCGGATGAATGTCAGCTACTGGGCTATCTGG | |
| | ACAAGGGAAAACGCAAGCGCAAAGAGAAAGCAGGTAGCTTGCAGTGGGCTTAC | |
| | ATGGCGATAGCTAGACTGGGCGGTTTTATGGACAGCAAGCGAACCGGAATTGC | |
| | CAGCTGGGGCGCCCTCTGGTAAGGTTGGGAAGCCCTGCAAAGTAAACTGGATG | |
| | GCTTTCTTGCCGCCAAGGATCTGATGGCGCAGGGGATCAAGATCTGATCAAGA | |
| | GACAGGATGAGGATCGTTTCGCATGATTGAACAAGATGGATTGCACGCAGGTT | |
| | CTCCGGCCGCTTGGGTGGAGAGGCTATTCGGCTATGACTGGGCACAACAGACA | |
| | ATCGGCTGCTCTGATGCCGCCGTGTTCCGGCTGTCAGCGCAGGGGCGCCCGGT | |
| | TCTTTTTGTCAAGACCGACCTGTCCGGTGCCCTGAATGAACTGCAGGACGAGG | |
| | CAGCGCGGCTATCGTGGCTGGCCACGACGGGCGTTCCTTGCGCAGCTGTGCTC | |
| | GACGTTGTCACTGAAGCGGGAAGGGACTGGCTGCTATTGGGCGAAGTGCCGGG | |
| | GCAGGATCTCCTGTCATCCCACCTTGCTCCTGCCGAGAAAGTATCCATCATGG | |
| | CTGATGCAATGCGGCGGCTGCATACGCTTGATCCGGCTACCTGCCCATTCGAC | |
| | CACCAAGCGAAACATCGCATCGAGCGAGCACGTACTCGGATGGAAGCCGGTCT | |
| | TGTCGATCAGGATGATCTGGACGAAGAGCATCAGGGGCTCGCGCCAGCCGAAC | |
| | TGTTCGCCAGGCTCAAGGCGCGCATGCCCGACGGCGAGGATCTCGTCGTGACC | |
| | CATGGCGATGCCTGCTTGCCGAATATCATGGTGGAAAATGGCCGCTTTTCTGG | |
| | ATTCATCGACTGTGGCCGGCTGGGTGTGGCGGACCGCTATCAGGACATAGCGT | |
| | TGGCTACCCGTGATATTGCTGAAGAGCTTGGCGGCGAATGGGCTGACCGCTTC | |
| | CTCGTGCTTTACGGTATCGCCGCTCCCGATTCGCAGCGCATCGCCTTCTATCG | |
| | CCTTCTTGACGAGTTCTTCTGAATTGAAAAAGGAAGAGTATGAGTATTCAACA | |
| | TTTCCGTGTCGCCCTTATTCCCTTTTTTGCGGCATTTTGCCTTCCTGTTTTTG | |
| | CTCACCCAGAAACGCTGGTGAAAGTAAAAGATGCTGAAGATCAGTTGGGTGCA | |
| | CGAGTGGGTTACATCGAACTGGATCTCAACAGCGGTAAGATCCTTGAGAGTTT | |
| | TCGCCCCGAAGAACGTTTTCCAATGATGAGCACTTTTAAAGTTCTGCTATGTG | |
| | GCGCGGTATTATCCCGTATTGACGCCGGGCAAGAGCAACTCGGTCGCCGCATA | |
| | CACTATTCTCAGAATGACTTGGTTGAGTACTCACCAGTCACAGAAAAGCATCT | |
| | TACGGATGGCATGACAGTAAGAGAATTATGCAGTGCTGCCATAACCATGAGTG | |
| | ATAACACTGCGGCCAACTTACTTCTGACAACGATCGGAGGACCGAAGGAGCTA | |
| | ACCGCTTTTTTGCACAACATGGGGGATCATGTAACTCGCCTTGATCGTTGGGA | |
| | ACCGGAGCTGAATGAAGCCATACCAAACGACGAGCGTGACACCACGATGCCTG | |
| | TAGCAATGGCAACAACGTTGCGCAAACTATTAACTGGCGAACTACTTACTCTA | |
| | GCTTCCCGGCAACAATTAATAGACTGGATGGAGGCGGATAAAGTTGCAGGACC | |
| | ACTTCTGCGCTCGGCCCTTCCGGCTGGCTGGTTTATTGCTGATAAATCTGGAG | |
| | CCGGTGAGCGTGGGTCTCGCGGTATCATTGCAGCACTGGGGCCAGATGGTAAG | |
| | CCCTCCCGTATCGTAGTTATCTACACGACGGGGAGTCAGGCAACTATGGATGA | |
| | ACGAAATAGACAGATCGCTGAGATAGGTGCCTCACTGATTAAGCATTGGTAAC | |
| | TGTCAGACCAAGTTTACTCATATATACTTTAGATTGATTTAAAACTTCATTTT | |
| | TAATTTAAAAGGATCTAGGTGAAGATCCTTTTTGATAATCTCATGACCAAAAT | |
| | CCCTTAACGTGAGTTTTCGTTCCACTGAGCGTCAGACCCCGTAGAAAAGATCA | |
| | AAGGATCTTCTTGAGATCCTTTTTTTCTGCGCGTAATCTGCTGCTTGCAAACA | |
| | AAAAAACCACCGCTACCAGCGGTGGTTTGTTTGCCGGATCAAGAGCTACCAAC | |
| | TCTTTTTCCGAAGGTAACTGGCTTCAGCAGAGCGCAGATACCAAATACTGTTC | |
| | TTCTAGTGTAGCCGTAGTTAGGCCACCACTTCAAGAACTCTGTAGCACCGCCT | |
| | ACATACCTCGCTCTGCTAATCCTGTTACCAGTGGCTGCTGCCAGTGGCGATAA | |
| | GTCGTGTCTTACCGGGTTGGACTCAAGACGATAGTTACCGGATAAGGCGCAGC | |
| | GGTCGGGCTGAACGGGGGGTTCGTGCACACAGCCCAGCTTGGAGCGAACGACC | |
| | TACACCGAACTGAGATACCTACAGCGTGAGCTATGAGAAAGCGCCACGCTTCC | |
| | CGAAGGGAGAAAGGCGGACAGGTATCCGGTAAGCGGCAGGGTCGGAACAGGAG | |
| | AGCGCACGAGGGAGCTTCCAGGGGGAAACGCCTGGTATCTTTATAGTCCTGTC | |
| | GGGTTTCGCCACCTCTGACTTGAGCGTCGATTTTTGTGATGCTCGTCAGGGGG | |
| | GCGGAGCCTATGGAAAAACGCCAGCAACGCGGCCTTTTTACGGTTCCTGGCCT | |
| | TTTGCTGGCCTTTTGCTCACATGTTCTTTCCTGCGTTATCCCCTGATTCTGTG | |
| | GATAACCGTATTACCGCCTTTGAGTGAGCTGATACCGCTCGCCGCAGCCGAAC | |
| | GACCGAGCGCAGCGAGTCAGTGAGCGAGG | |
| MB8203 | AAGCGGAAGAGCGCCCAATACGCAAACCGCCTCTCCCCGCGCGTTGGCCGATT | 10 |
| | CATTAATGCAGCTGGCACGACAGGTTTCCCGACTGGAAAGCGGGCAGTGAGCG | |
| | CAACGCAATTAATGTGAGTTAGCTCACTCATTAGGCACCCCAGGCTTTACACT | |
| | TTATGCTTCCGGCTCGTATGTTGTGTGGAATTGTGAGCGGATAACAATTTCAC | |
| | ACAGGAAACAGCTATGACCATGATTACGCCAAGCTTGGTACCAGAGACGGGTT | |
| | GGCGGCGTATTTGTGTCCCAAAAAACAGCCCCAATTGCCCCAATTGACCCCAA | |
| | ATTGACCCAGTAGCGGGCCCAACCCCGGCGAGAGCCCCCTTCACCCCACATAT | |
| | CAAACCTCCCCCGGTTCCCACACTTGCCGTTAAGGGCGTAGGGTACTGCAGTC | |
| | TGGAATCTACGCTTGTTCAGACTTTGTACTAGTTTCTTTGTCTGGCCATCCGG | |
| | GTAACCCATGCCGGACGCAAAATAGACTACTGAAAATTTTTTTGCTTTGTGGT | |
| | TGGGACTTTAGCCAAGGGTATAAAAGACCACCGTCCCCGAATTACCTTTCCTC | |
| | TTCTTTTCTCTCTCTCCTTGTCAACTCACACCCGAAATCGTTAAGCATTTCCT | |
| | TCTGAGTATAAGAATCATTCGCTAGCCACAAAAATGATGGCTCCCTCCATTCG | |
| | AAAGTTCTTTGCTGGTGGTGTGTGTCGAACTAACGTTCAGCTTCCCGGTAAGG | |
| | TGGTTGTCATCACTGGTGCCAACACCGGCATTGGCAAGGAGACTGCCCGAGAG | |
| | CTCGCTTCCCGAGGAGCCCGAGTTTACATTGCTTGCCGAGATGTTCTGAAGGG | |
| | CGAGTCTGCTGCCTCTGAGATTCGAGTTGACACTAAGAACTCCCAGGTGCTCG | |
| | TGCGAAAGCTCGACCTTTCCGACACTAAGTCTATCCGAGCCTTTGCTGAGGGC | |
| | TTTCTCGCTGAGGAGAAGCAGCTTCACATTCTGATTAACAACGCTGGAGTTAT | |
| | GATGTGTCCTTACTCTAAGACTGCTGATGGTTTCGAGACTCACCTCGGAGTCA | |
| | ACCACCTGGGCCACTTCCTCCTCACCTACCTGCTCCTGGAGCGACTCAAGGTG | |
| | TCTGCCCCTGCCCGAGTGGTTAACGTTTCCTCCGTGGCTCACCACATTGGCAA | |
| | GATTCCCTTCCACGACCTCCAGTCCGAGAAGCGATACTCCCGAGGTTTTGCTT | |
| | ACTGCCACTCCAAGCTGGCCAACGTTCTCTTTACCCGAGAGCTGGCCAAGCGA | |
| | CTCCAGGGAACCGGCGTCACCACCTACGCCGTTCACCCCGGTGTCGTCCGATC | |
| | CGAGCTGGTCCGACACTCCTCCCTGCTCTGCCTGCTCTGGCGACTCTTCTCCC | |
| | CCTTCGTTAAGACCGCCCGAGAGGGTGCCCAGACCTCCCTGCACTGCGCCCTG | |
| | GCTGAGGGCCTGGAGCCCCTGTCTGGCAAGTACTTCTCTGACTGCAAGCGAAC | |
| | CTGGGTGTCTCCCCGAGCTCGAAACAACAAGACTGCCGAGCGACTCTGGAACG | |
| | TTTCCTGTGAGCTTCTCGGTATTCGATGGGAGTAGACGCGTGCAATTAACAGA | |
| | TAGTTTGCCGGTGATAATTCTCTTAACCTCCCACACTCCTTTGACATAACGAT | |
| | TTATGTAACGAAACTGAAATTTGACCAGATATTGTTGTAAATAGAAAATCTGG | |
| | CTTGTAGGTGGCAAACTAGTGGTGTGTTCTGTGGAGCATTCTCACTTTTGGTA | |
| | AACGACATTGCTTCAAGTGCAGCGGAATCAAAAAGTATAAAGTGGGCAGCGAG | |
| | TATACCTGTACAGACTGTAGGCGATAACTCAATCCAATTACCCCCCACAACAT | |
| | GACTGGCCAAACTGATCTCAAGACTTTATTGAAATCAGCAACACCGATTCTCA | |
| | ATGAAGGCACATACTTCTTCTGCAACATTCACTTGACGCCTAAAGTTGGTGAG | |
| | AAATGGACCGACAAGACATATTCTGCTATCCACGGACTGTTGCCTGTGTCGGT | |
| | GGCTACAATACGTGAGTCAGAAGGGCTGACGGTGGTGGTTCCCAAGGAAAAGG | |
| | TCGACGAGTATCTGTCTGACTCGTCATTGCCGCCTTTGGAGTACGACTCCAAC | |
| | TATGAGTGTGCTTGGATCACTTTGACGATACATTCTTCGTTGGAGGCTGTGGG | |
| | TCTGACAGCTGCGTTTTCGGCGCGGTTGGCCGACAACAATATCAGCTGCAACG | |
| | TCATTGCTGGCTTTCATCATGATCACATTTTTGTCGGCAAAGGCGACGCCCAG | |
| | AGAGCCATTGACGTTCTTTCTAATTTGGACCGATAGCCGTATAGTCCAGTCTA | |
| | TCTATAAGTTCAACTAACTCGTAACTATTACCATAACATATACTTCACTGCCC | |
| | CAGATAAGGTTCCGATAAAAAGTTCTGCAGACTAAATTTATTTCAGTCTCCTC | |
| | TTCACCACCAAAATGCCCTCCTACGAAGCTCGAGCTAACGTCCACAAGTCCGC | |
| | CTTTGCCGCTCGAGTGCTCAAGCTCGTGGCAGCCAAGAAAACCAACCTGTGTG | |
| | CTTCTCTGGATGTTACCACCACCAAGGAGCTCATTGAGCTTGCCGATAAGGTC | |
| | GGACCTTATGTGTGCATGATCAAGACCCATATCGACATCATTGACGACTTCAC | |
| | CTACGCCGGCACTGTGCTCCCCCTCAAGGAACTTGCTCTTAAGCACGGTTTCT | |
| | TCCTGTTCGAGGACAGAAAGTTCGCAGATATTGGCAACACTGTCAAGCACCAG | |
| | TACAAGAACGGTGTCTACCGAATCGCCGAGTGGTCCGATATCACCAACGCCCA | |
| | CGGTGTACCCGGAACCGGAATCATTGCTGGCCTGCGAGCTGGTGCCGAGGAAA | |
| | CTGTCTCTGAACAGAAGAAGGAGGACGTCTCTGACTACGAGAACTCCCAGTAC | |
| | AAGGAGTTCCTGGTCCCCTCTCCCAACGAGAAGCTGGCCAGAGGTCTGCTCAT | |
| | GCTGGCCGAGCTGTCTTGCAAGGGCTCTCTGGCCACTGGCGAGTACTCCAAGC | |
| | AGACCATTGAGCTTGCCCGATCCGACCCCGAGTTTGTGGTTGGCTTCATTGCC | |
| | CAGAACCGACCTAAGGGCGACTCTGAGGACTGGCTTATTCTGACCCCCGGGGT | |
| | GGGTCTTGACGACAAGGGAGACGCTCTCGGACAGCAGTACCGAACTGTTGAGG | |
| | ATGTCATGTCTACCGGAACGGATATCATAATTGTCGGCCGAGGTCTGTACGGC | |
| | CAGAACCGAGATCCTATTGAGGAGGCCAAGCGATACCAGAAGGCTGGCTGGGA | |
| | GGCTTACCAGAAGATTAACTGTTAGAGGTTAGACTATGGATATGTAATTTAAC | |
| | TGTGTATATAGAGAGCGTGCAAGTATGGAGCGCTTGTTCAGCTTGTATGATGG | |
| | TCAGACGACCTGTCTGATCGAGTATGTATGATACTGCACAACCTGTGTATCCG | |
| | CATGATCTGTCCAATGGGGCATGTTGTTGTGTTTCTCGATACGGAGATGCTGG | |
| | GTACAAGTAGCTAATACGATTGAACTACTTATACTTATATGAGGCTTGAAGAA | |
| | AGCTGACTTGTGTATGACTTATTCTCAACTACATCCCCAGTCACAATACCACC | |
| | ACTGCACTACCACTACACCTCGAGCATGCATCTAGTGGTGTGTTCTGTGGAGC | |
| | ATTCTCACTTTTGGTAAACGACATTGCTTCAAGTGCAGCGGAATCAAAAAGTA | |
| | TAAAGTGGGCAGCGAGTATACCTGTACAGACTGTAGGCGATAACTCAATCCAA | |
| | TTACCCCCCACAACATGACTGGCCAAACTGATCTCAAGACTTTATTGAAATCA | |
| | GCAACACCGATTCTCAATGAAGGCACATACTTCTTCTGCAACATTCACTTGAC | |
| | GCCTAAAGTTGGTGAGAAATGGACCGACAAGACATATTCTGCTATCCACGGAC | |
| | TGTTGCCTGTGTCGGTGGCTACAATACGTGAGTCAGAAGGGCTGACGGTGGTG | |
| | GTTCCCAAGGAAAAGGTCGACGAGTATCTGTCTGACTCGTCATTGCCGCCTTT | |
| | GGAGTACGACTCCAACTATGAGTGTGCTTGGATCACTTTGACGATACATTCTT | |
| | CGTTGGAGGCTGTGGGTCTGACAGCTGCGTTTTCGGCGCGGTTGGCCGACAAC | |
| | AATATCAGCTGCAACGTCATTGCTGGCTTTCATCATGATCACATTTTTGTCGG | |
| | CAAAGGCGACGCCCAGAGAGCCATTGACGTTCTTTCTAATTTGGACCGATAGC | |
| | CGTATAGTCCAGTCTATCTATAAGTTCAACTAACTCGTAACTATTACCATAAC | |
| | ATATACTTCACTGCCCCAGATAAGGTTCCGATAAAAAGTTCTGCAGACTAAAT | |
| | TTATTTCAGTCTCCTCTTCACCACCAAAATGCCCTCCTACGAAGCTCGAGCTA | |
| | ACGTCCACAAGTCCGCCTTTGCCGCTCGAGTGCTCAAGCTCGTGGCAGCCAAG | |
| | AAAACCAACCTGTGTGCTTCTCTGGATGTTACCACCACCAAGGGGCCGCTCGA | |
| | GCATGCATCTAGAGGGCCCAATTCGCCCTATAGTGAGTCGTATTACAATTCAC | |
| | TGGCCGTCGTTTTACAACGTCGTGACTGGGAAAACCCTGGCGTTACCCAACTT | |
| | AATCGCCTTGCAGCACATCCCCCTTTCGCCAGCTGGCGTAATAGCGAAGAGGC | |
| | CCGCACCGATCGCCCTTCCCAACAGTTGCGCAGCCTGAATGGCGAATGGACGC | |
| | GCCCTGTAGCGGCGCATTAAGCGCGGCGGGTGTGGTGGTTACGCGCAGCGTGA | |
| | CCGCTACACTTGCCAGCGCCCTAGCGCCCGCTCCTTTCGCTTTCTTCCCTTCC | |
| | TTTCTCGCCACGTTCGCCGGCTTTCCCCGTCAAGCTCTAAATCGGGGGCTCCC | |
| | TTTAGGGTTCCGATTTAGTGCTTTACGGCACCTCGACCCCAAAAAACTTGATT | |
| | AGGGTGATGGTTCACGTAGTGGGCCATCGCCCTGATAGACGGTTTTTCGCCCT | |
| | TTGACGTTGGAGTCCACGTTCTTTAATAGTGGACTCTTGTTCCAAACTGGAAC | |
| | AACACTCAACCCTATCTCGGTCTATTCTTTTGATTTATAAGGGATTTTGCCGA | |
| | TTTCGGCCTATTGGTTAAAAAATGAGCTGATTTAACAAAAATTTAACGCGAAT | |
| | TTTAACAAAATTCAGGGCGCAAGGGCTGCTAAAGGAAGCGGAACACGTAGAAA | |
| | GCCAGTCCGCAGAAACGGTGCTGACCCCGGATGAATGTCAGCTACTGGGCTAT | |
| | CTGGACAAGGGAAAACGCAAGCGCAAAGAGAAAGCAGGTAGCTTGCAGTGGGC | |
| | TTACATGGCGATAGCTAGACTGGGCGGTTTTATGGACAGCAAGCGAACCGGAA | |
| | TTGCCAGCTGGGGCGCCCTCTGGTAAGGTTGGGAAGCCCTGCAAAGTAAACTG | |
| | GATGGCTTTCTTGCCGCCAAGGATCTGATGGCGCAGGGGATCAAGATCTGATC | |
| | AAGAGACAGGATGAGGATCGTTTCGCATGATTGAACAAGATGGATTGCACGCA | |
| | GGTTCTCCGGCCGCTTGGGTGGAGAGGCTATTCGGCTATGACTGGGCACAACA | |
| | GACAATCGGCTGCTCTGATGCCGCCGTGTTCCGGCTGTCAGCGCAGGGGCGCC | |
| | CGGTTCTTTTTGTCAAGACCGACCTGTCCGGTGCCCTGAATGAACTGCAGGAC | |
| | GAGGCAGCGCGGCTATCGTGGCTGGCCACGACGGGCGTTCCTTGCGCAGCTGT | |
| | GCTCGACGTTGTCACTGAAGCGGGAAGGGACTGGCTGCTATTGGGCGAAGTGC | |
| | CGGGGCAGGATCTCCTGTCATCCCACCTTGCTCCTGCCGAGAAAGTATCCATC | |
| | ATGGCTGATGCAATGCGGCGGCTGCATACGCTTGATCCGGCTACCTGCCCATT | |
| | CGACCACCAAGCGAAACATCGCATCGAGCGAGCACGTACTCGGATGGAAGCCG | |
| | GTCTTGTCGATCAGGATGATCTGGACGAAGAGCATCAGGGGCTCGCGCCAGCC | |
| | GAACTGTTCGCCAGGCTCAAGGCGCGCATGCCCGACGGCGAGGATCTCGTCGT | |
| | GACCCATGGCGATGCCTGCTTGCCGAATATCATGGTGGAAAATGGCCGCTTTT | |
| | CTGGATTCATCGACTGTGGCCGGCTGGGTGTGGCGGACCGCTATCAGGACATA | |
| | GCGTTGGCTACCCGTGATATTGCTGAAGAGCTTGGCGGCGAATGGGCTGACCG | |
| | CTTCCTCGTGCTTTACGGTATCGCCGCTCCCGATTCGCAGCGCATCGCCTTCT | |
| | ATCGCCTTCTTGACGAGTTCTTCTGAATTGAAAAAGGAAGAGTATGAGTATTC | |
| | AACATTTCCGTGTCGCCCTTATTCCCTTTTTTGCGGCATTTTGCCTTCCTGTT | |
| | TTTGCTCACCCAGAAACGCTGGTGAAAGTAAAAGATGCTGAAGATCAGTTGGG | |
| | TGCACGAGTGGGTTACATCGAACTGGATCTCAACAGCGGTAAGATCCTTGAGA | |
| | GTTTTCGCCCCGAAGAACGTTTTCCAATGATGAGCACTTTTAAAGTTCTGCTA | |
| | TGTGGCGCGGTATTATCCCGTATTGACGCCGGGCAAGAGCAACTCGGTCGCCG | |
| | CATACACTATTCTCAGAATGACTTGGTTGAGTACTCACCAGTCACAGAAAAGC | |
| | ATCTTACGGATGGCATGACAGTAAGAGAATTATGCAGTGCTGCCATAACCATG | |
| | AGTGATAACACTGCGGCCAACTTACTTCTGACAACGATCGGAGGACCGAAGGA | |
| | GCTAACCGCTTTTTTGCACAACATGGGGGATCATGTAACTCGCCTTGATCGTT | |
| | GGGAACCGGAGCTGAATGAAGCCATACCAAACGACGAGCGTGACACCACGATG | |
| | CCTGTAGCAATGGCAACAACGTTGCGCAAACTATTAACTGGCGAACTACTTAC | |
| | TCTAGCTTCCCGGCAACAATTAATAGACTGGATGGAGGCGGATAAAGTTGCAG | |
| | GACCACTTCTGCGCTCGGCCCTTCCGGCTGGCTGGTTTATTGCTGATAAATCT | |
| | GGAGCCGGTGAGCGTGGGTCTCGCGGTATCATTGCAGCACTGGGGCCAGATGG | |
| | TAAGCCCTCCCGTATCGTAGTTATCTACACGACGGGGAGTCAGGCAACTATGG | |
| | ATGAACGAAATAGACAGATCGCTGAGATAGGTGCCTCACTGATTAAGCATTGG | |
| | TAACTGTCAGACCAAGTTTACTCATATATACTTTAGATTGATTTAAAACTTCA | |
| | TTTTTAATTTAAAAGGATCTAGGTGAAGATCCTTTTTGATAATCTCATGACCA | |
| | AAATCCCTTAACGTGAGTTTTCGTTCCACTGAGCGTCAGACCCCGTAGAAAAG | |
| | ATCAAAGGATCTTCTTGAGATCCTTTTTTTCTGCGCGTAATCTGCTGCTTGCA | |
| | AACAAAAAAACCACCGCTACCAGCGGTGGTTTGTTTGCCGGATCAAGAGCTAC | |
| | CAACTCTTTTTCCGAAGGTAACTGGCTTCAGCAGAGCGCAGATACCAAATACT | |
| | GTTCTTCTAGTGTAGCCGTAGTTAGGCCACCACTTCAAGAACTCTGTAGCACC | |
| | GCCTACATACCTCGCTCTGCTAATCCTGTTACCAGTGGCTGCTGCCAGTGGCG | |
| | ATAAGTCGTGTCTTACCGGGTTGGACTCAAGACGATAGTTACCGGATAAGGCG | |
| | CAGCGGTCGGGCTGAACGGGGGGTTCGTGCACACAGCCCAGCTTGGAGCGAAC | |
| | GACCTACACCGAACTGAGATACCTACAGCGTGAGCTATGAGAAAGCGCCACGC | |
| | TTCCCGAAGGGAGAAAGGCGGACAGGTATCCGGTAAGCGGCAGGGTCGGAACA | |
| | GGAGAGCGCACGAGGGAGCTTCCAGGGGGAAACGCCTGGTATCTTTATAGTCC | |
| | TGTCGGGTTTCGCCACCTCTGACTTGAGCGTCGATTTTTGTGATGCTCGTCAG | |
| | GGGGGCGGAGCCTATGGAAAAACGCCAGCAACGCGGCCTTTTTACGGTTCCTG | |
| | GCCTTTTGCTGGCCTTTTGCTCACATGTTCTTTCCTGCGTTATCCCCTGATTC | |
| | TGTGGATAACCGTATTACCGCCTTTGAGTGAGCTGATACCGCTCGCCGCAGCC | |
| | GAACGACCGAGCGCAGCGAGTCAGTGAGCGAGG | |
| MB9894 | AAGCGGAAGAGCGCCCAATACGCAAACCGCCTCTCCCCGCGCGTTGGCCGATT | 11 |
| | CATTAATGCAGCTGGCACGACAGGTTTCCCGACTGGAAAGCGGGCAGTGAGCG | |
| | CAACGCAATTAATGTGAGTTAGCTCACTCATTAGGCACCCCAGGCTTTACACT | |
| | TTATGCTTCCGGCTCGTATGTTGTGTGGAATTGTGAGCGGATAACAATTTCAC | |
| | ACAGGAAACAGCTATGACCATGATTACGCCAAGCTTGGTACCAGAGACGGGTT | |
| | GGCGGCGTATTTGTGTCCCAAAAAACAGCCCCAATTGCCCCAATTGACCCCAA | |
| | ATTGACCCAGTAGCGGGCCCAACCCCGGCGAGAGCCCCCTTCACCCCACATAT | |
| | CAAACCTCCCCCGGTTCCCACACTTGCCGTTAAGGGCGTAGGGTACTGCAGTC | |
| | TGGAATCTACGCTTGTTCAGACTTTGTACTAGTTTCTTTGTCTGGCCATCCGG | |
| | GTAACCCATGCCGGACGCAAAATAGACTACTGAAAATTTTTTTGCTTTGTGGT | |
| | TGGGACTTTAGCCAAGGGTATAAAAGACCACCGTCCCCGAATTACCTTTCCTC | |
| | TTCTTTTCTCTCTCTCCTTGTCAACTCACACCCGAAATCGTTAAGCATTTCCT | |
| | TCTGAGTATAAGAATCATTCGCTAGCCCACAAAAATGACCCGATCCAAGTTCA | |
| | CCGAGTACAAGCAGGTTCTCCGAAACTCTTGTGTTGGCAAGCCCCAGTACACT | |
| | GAGAAGGATTACCCCGATCTCGACGGAAAGAACTTTCTCGTCACTGGTGCTAC | |
| | TGGTGGAGTTGGTCTGGAGGCCACTAAGCTGCTCCTTGAGAAGAAGTCTCACG | |
| | TTATTATGGTTGGCCGATCTAAGACTAAGTCTCAGTCTACCCTTGACGAGCTT | |
| | CAGAAGACTTACTCTCACGGTACTTTCGATTTCGTTGAGGCTGATCTCTCTGA | |
| | CCTTACCACTGTTGAGCGAGCCGGAGAGTACATTCGATCTAAGTACACTACCC | |
| | TTGACGGTGCCATTCTTAACGCTGGAGTCATGGCTCCCCCCTACTCTCTCACT | |
| | CCCCAGGGTCACGAGTCTCAGTGGGGTATTAACGTTGTTGCTCACTTTCTCCT | |
| | GTCTAAGTACATCTCTCCTGCTCTGATTTCTGCTGCTCAGACTGCCCCCAAGG | |
| | ACACCGTTCGACTTGTTTGGGTGTCTTCTTCTGTCGTTGCCATGTCTCCTTAC | |
| | GAGGGAGGTATTAAGTTTGACGATATTAACCACTCTAAGGTCAAGAACCCTTC | |
| | TCCTTGGACCCTTTACTCTCAGTCTAAGATTGGTGATGCTTACCTGGCTTACC | |
| | TTTGGTCTAAGCACCACCCCGATTCTGGAGTCCTTTCTGTCTCTCTGGACCCC | |
| | GGTAACCTGGCTTCTGATCTCTCTCGACACACTTCTTGGCTTTCTTCTATTAA | |
| | GAACTACGTTCTTTACCCTCCCAAGTACGGTGCCTACACTGAGCTGTCTGCTC | |
| | TTCTCAACCCTTCCGTTAAGAACAACGAGCACCTTATTCCCTGGGGTATTGAG | |
| | GGACACCTTCGACAGGATGTCGACGATGGTCGACGAGGAAAGGACGGAGAGGA | |
| | GCTCTGGCAGGGTCTTAACAAGGATGTCGAGGGTTTCTTTAAGGAGGAGTAAA | |
| | CGCGTGCAATTAACAGATAGTTTGCCGGTGATAATTCTCTTAACCTCCCACAC | |
| | TCCTTTGACATAACGATTTATGTAACGAAACTGAAATTTGACCAGATATTGTT | |
| | GTAAATAGAAAATCTGGCTTGTAGGTGGCAAACTAGTGGTGTGTTCTGTGGAG | |
| | CATTCTCACTTTTGGTAAACGACATTGCTTCAAGTGCAGCGGAATCAAAAAGT | |
| | ATAAAGTGGGCAGCGAGTATACCTGTACAGACTGTAGGCGATAACTCAATCCA | |
| | ATTACCCCCCACAACATGACTGGCCAAACTGATCTCAAGACTTTATTGAAATC | |
| | AGCAACACCGATTCTCAATGAAGGCACATACTTCTTCTGCAACATTCACTTGA | |
| | CGCCTAAAGTTGGTGAGAAATGGACCGACAAGACATATTCTGCTATCCACGGA | |
| | CTGTTGCCTGTGTCGGTGGCTACAATACGTGAGTCAGAAGGGCTGACGGTGGT | |
| | GGTTCCCAAGGAAAAGGTCGACGAGTATCTGTCTGACTCGTCATTGCCGCCTT | |
| | TGGAGTACGACTCCAACTATGAGTGTGCTTGGATCACTTTGACGATACATTCT | |
| | TCGTTGGAGGCTGTGGGTCTGACAGCTGCGTTTTCGGCGCGGTTGGCCGACAA | |
| | CAATATCAGCTGCAACGTCATTGCTGGCTTTCATCATGATCACATTTTTGTCG | |
| | GCAAAGGCGACGCCCAGAGAGCCATTGACGTTCTTTCTAATTTGGACCGATAG | |
| | CCGTATAGTCCAGTCTATCTATAAGTTCAACTAACTCGTAACTATTACCATAA | |
| | CATATACTTCACTGCCCCAGATAAGGTTCCGATAAAAAGTTCTGCAGACTAAA | |
| | TTTATTTCAGTCTCCTCTTCACCACCAAAATGCCCTCCTACGAAGCTCGAGCT | |
| | AACGTCCACAAGTCCGCCTTTGCCGCTCGAGTGCTCAAGCTCGTGGCAGCCAA | |
| | GAAAACCAACCTGTGTGCTTCTCTGGATGTTACCACCACCAAGGAGCTCATTG | |
| | AGCTTGCCGATAAGGTCGGACCTTATGTGTGCATGATCAAGACCCATATCGAC | |
| | ATCATTGACGACTTCACCTACGCCGGCACTGTGCTCCCCCTCAAGGAACTTGC | |
| | TCTTAAGCACGGTTTCTTCCTGTTCGAGGACAGAAAGTTCGCAGATATTGGCA | |
| | ACACTGTCAAGCACCAGTACAAGAACGGTGTCTACCGAATCGCCGAGTGGTCC | |
| | GATATCACCAACGCCCACGGTGTACCCGGAACCGGAATCATTGCTGGCCTGCG | |
| | AGCTGGTGCCGAGGAAACTGTCTCTGAACAGAAGAAGGAGGACGTCTCTGACT | |
| | ACGAGAACTCCCAGTACAAGGAGTTCCTGGTCCCCTCTCCCAACGAGAAGCTG | |
| | GCCAGAGGTCTGCTCATGCTGGCCGAGCTGTCTTGCAAGGGCTCTCTGGCCAC | |
| | TGGCGAGTACTCCAAGCAGACCATTGAGCTTGCCCGATCCGACCCCGAGTTTG | |
| | TGGTTGGCTTCATTGCCCAGAACCGACCTAAGGGCGACTCTGAGGACTGGCTT | |
| | ATTCTGACCCCCGGGGTGGGTCTTGACGACAAGGGAGACGCTCTCGGACAGCA | |
| | GTACCGAACTGTTGAGGATGTCATGTCTACCGGAACGGATATCATAATTGTCG | |
| | GCCGAGGTCTGTACGGCCAGAACCGAGATCCTATTGAGGAGGCCAAGCGATAC | |
| | CAGAAGGCTGGCTGGGAGGCTTACCAGAAGATTAACTGTTAGAGGTTAGACTA | |
| | TGGATATGTAATTTAACTGTGTATATAGAGAGCGTGCAAGTATGGAGCGCTTG | |
| | TTCAGCTTGTATGATGGTCAGACGACCTGTCTGATCGAGTATGTATGATACTG | |
| | CACAACCTGTGTATCCGCATGATCTGTCCAATGGGGCATGTTGTTGTGTTTCT | |
| | CGATACGGAGATGCTGGGTACAAGTAGCTAATACGATTGAACTACTTATACTT | |
| | ATATGAGGCTTGAAGAAAGCTGACTTGTGTATGACTTATTCTCAACTACATCC | |
| | CCAGTCACAATACCACCACTGCACTACCACTACACCTCGAGCATGCATCTAGT | |
| | GGTGTGTTCTGTGGAGCATTCTCACTTTTGGTAAACGACATTGCTTCAAGTGC | |
| | AGCGGAATCAAAAAGTATAAAGTGGGCAGCGAGTATACCTGTACAGACTGTAG | |
| | GCGATAACTCAATCCAATTACCCCCCACAACATGACTGGCCAAACTGATCTCA | |
| | AGACTTTATTGAAATCAGCAACACCGATTCTCAATGAAGGCACATACTTCTTC | |
| | TGCAACATTCACTTGACGCCTAAAGTTGGTGAGAAATGGACCGACAAGACATA | |
| | TTCTGCTATCCACGGACTGTTGCCTGTGTCGGTGGCTACAATACGTGAGTCAG | |
| | AAGGGCTGACGGTGGTGGTTCCCAAGGAAAAGGTCGACGAGTATCTGTCTGAC | |
| | TCGTCATTGCCGCCTTTGGAGTACGACTCCAACTATGAGTGTGCTTGGATCAC | |
| | TTTGACGATACATTCTTCGTTGGAGGCTGTGGGTCTGACAGCTGCGTTTTCGG | |
| | CGCGGTTGGCCGACAACAATATCAGCTGCAACGTCATTGCTGGCTTTCATCAT | |
| | GATCACATTTTTGTCGGCAAAGGCGACGCCCAGAGAGCCATTGACGTTCTTTC | |
| | TAATTTGGACCGATAGCCGTATAGTCCAGTCTATCTATAAGTTCAACTAACTC | |
| | GTAACTATTACCATAACATATACTTCACTGCCCCAGATAAGGTTCCGATAAAA | |
| | AGTTCTGCAGACTAAATTTATTTCAGTCTCCTCTTCACCACCAAAATGCCCTC | |
| | CTACGAAGCTCGAGCTAACGTCCACAAGTCCGCCTTTGCCGCTCGAGTGCTCA | |
| | AGCTCGTGGCAGCCAAGAAAACCAACCTGTGTGCTTCTCTGGATGTTACCACC | |
| | ACCAAGGGGCCGCTCGAGCATGCATCTAGAGGGCCCAATTCGCCCTATAGTGA | |
| | GTCGTATTACAATTCACTGGCCGTCGTTTTACAACGTCGTGACTGGGAAAACC | |
| | CTGGCGTTACCCAACTTAATCGCCTTGCAGCACATCCCCCTTTCGCCAGCTGG | |
| | CGTAATAGCGAAGAGGCCCGCACCGATCGCCCTTCCCAACAGTTGCGCAGCCT | |
| | GAATGGCGAATGGACGCGCCCTGTAGCGGCGCATTAAGCGCGGCGGGTGTGGT | |
| | GGTTACGCGCAGCGTGACCGCTACACTTGCCAGCGCCCTAGCGCCCGCTCCTT | |
| | TCGCTTTCTTCCCTTCCTTTCTCGCCACGTTCGCCGGCTTTCCCCGTCAAGCT | |
| | CTAAATCGGGGGCTCCCTTTAGGGTTCCGATTTAGTGCTTTACGGCACCTCGA | |
| | CCCCAAAAAACTTGATTAGGGTGATGGTTCACGTAGTGGGCCATCGCCCTGAT | |
| | AGACGGTTTTTCGCCCTTTGACGTTGGAGTCCACGTTCTTTAATAGTGGACTC | |
| | TTGTTCCAAACTGGAACAACACTCAACCCTATCTCGGTCTATTCTTTTGATTT | |
| | ATAAGGGATTTTGCCGATTTCGGCCTATTGGTTAAAAAATGAGCTGATTTAAC | |
| | AAAAATTTAACGCGAATTTTAACAAAATTCAGGGCGCAAGGGCTGCTAAAGGA | |
| | AGCGGAACACGTAGAAAGCCAGTCCGCAGAAACGGTGCTGACCCCGGATGAAT | |
| | GTCAGCTACTGGGCTATCTGGACAAGGGAAAACGCAAGCGCAAAGAGAAAGCA | |
| | GGTAGCTTGCAGTGGGCTTACATGGCGATAGCTAGACTGGGCGGTTTTATGGA | |
| | CAGCAAGCGAACCGGAATTGCCAGCTGGGGCGCCCTCTGGTAAGGTTGGGAAG | |
| | CCCTGCAAAGTAAACTGGATGGCTTTCTTGCCGCCAAGGATCTGATGGCGCAG | |
| | GGGATCAAGATCTGATCAAGAGACAGGATGAGGATCGTTTCGCATGATTGAAC | |
| | AAGATGGATTGCACGCAGGTTCTCCGGCCGCTTGGGTGGAGAGGCTATTCGGC | |
| | TATGACTGGGCACAACAGACAATCGGCTGCTCTGATGCCGCCGTGTTCCGGCT | |
| | GTCAGCGCAGGGGCGCCCGGTTCTTTTTGTCAAGACCGACCTGTCCGGTGCCC | |
| | TGAATGAACTGCAGGACGAGGCAGCGCGGCTATCGTGGCTGGCCACGACGGGC | |
| | GTTCCTTGCGCAGCTGTGCTCGACGTTGTCACTGAAGCGGGAAGGGACTGGCT | |
| | GCTATTGGGCGAAGTGCCGGGGCAGGATCTCCTGTCATCCCACCTTGCTCCTG | |
| | CCGAGAAAGTATCCATCATGGCTGATGCAATGCGGCGGCTGCATACGCTTGAT | |
| | CCGGCTACCTGCCCATTCGACCACCAAGCGAAACATCGCATCGAGCGAGCACG | |
| | TACTCGGATGGAAGCCGGTCTTGTCGATCAGGATGATCTGGACGAAGAGCATC | |
| | AGGGGCTCGCGCCAGCCGAACTGTTCGCCAGGCTCAAGGCGCGCATGCCCGAC | |
| | GGCGAGGATCTCGTCGTGACCCATGGCGATGCCTGCTTGCCGAATATCATGGT | |
| | GGAAAATGGCCGCTTTTCTGGATTCATCGACTGTGGCCGGCTGGGTGTGGCGG | |
| | ACCGCTATCAGGACATAGCGTTGGCTACCCGTGATATTGCTGAAGAGCTTGGC | |
| | GGCGAATGGGCTGACCGCTTCCTCGTGCTTTACGGTATCGCCGCTCCCGATTC | |
| | GCAGCGCATCGCCTTCTATCGCCTTCTTGACGAGTTCTTCTGAATTGAAAAAG | |
| | GAAGAGTATGAGTATTCAACATTTCCGTGTCGCCCTTATTCCCTTTTTTGCGG | |
| | CATTTTGCCTTCCTGTTTTTGCTCACCCAGAAACGCTGGTGAAAGTAAAAGAT | |
| | GCTGAAGATCAGTTGGGTGCACGAGTGGGTTACATCGAACTGGATCTCAACAG | |
| | CGGTAAGATCCTTGAGAGTTTTCGCCCCGAAGAACGTTTTCCAATGATGAGCA | |
| | CTTTTAAAGTTCTGCTATGTGGCGCGGTATTATCCCGTATTGACGCCGGGCAA | |
| | GAGCAACTCGGTCGCCGCATACACTATTCTCAGAATGACTTGGTTGAGTACTC | |
| | ACCAGTCACAGAAAAGCATCTTACGGATGGCATGACAGTAAGAGAATTATGCA | |
| | GTGCTGCCATAACCATGAGTGATAACACTGCGGCCAACTTACTTCTGACAACG | |
| | ATCGGAGGACCGAAGGAGCTAACCGCTTTTTTGCACAACATGGGGGATCATGT | |
| | AACTCGCCTTGATCGTTGGGAACCGGAGCTGAATGAAGCCATACCAAACGACG | |
| | AGCGTGACACCACGATGCCTGTAGCAATGGCAACAACGTTGCGCAAACTATTA | |
| | ACTGGCGAACTACTTACTCTAGCTTCCCGGCAACAATTAATAGACTGGATGGA | |
| | GGCGGATAAAGTTGCAGGACCACTTCTGCGCTCGGCCCTTCCGGCTGGCTGGT | |
| | TTATTGCTGATAAATCTGGAGCCGGTGAGCGTGGGTCTCGCGGTATCATTGCA | |
| | GCACTGGGGCCAGATGGTAAGCCCTCCCGTATCGTAGTTATCTACACGACGGG | |
| | GAGTCAGGCAACTATGGATGAACGAAATAGACAGATCGCTGAGATAGGTGCCT | |
| | CACTGATTAAGCATTGGTAACTGTCAGACCAAGTTTACTCATATATACTTTAG | |
| | ATTGATTTAAAACTTCATTTTTAATTTAAAAGGATCTAGGTGAAGATCCTTTT | |
| | TGATAATCTCATGACCAAAATCCCTTAACGTGAGTTTTCGTTCCACTGAGCGT | |
| | CAGACCCCGTAGAAAAGATCAAAGGATCTTCTTGAGATCCTTTTTTTCTGCGC | |
| | GTAATCTGCTGCTTGCAAACAAAAAAACCACCGCTACCAGCGGTGGTTTGTTT | |
| | GCCGGATCAAGAGCTACCAACTCTTTTTCCGAAGGTAACTGGCTTCAGCAGAG | |
| | CGCAGATACCAAATACTGTTCTTCTAGTGTAGCCGTAGTTAGGCCACCACTTC | |
| | AAGAACTCTGTAGCACCGCCTACATACCTCGCTCTGCTAATCCTGTTACCAGT | |
| | GGCTGCTGCCAGTGGCGATAAGTCGTGTCTTACCGGGTTGGACTCAAGACGAT | |
| | AGTTACCGGATAAGGCGCAGCGGTCGGGCTGAACGGGGGGTTCGTGCACACAG | |
| | CCCAGCTTGGAGCGAACGACCTACACCGAACTGAGATACCTACAGCGTGAGCT | |
| | ATGAGAAAGCGCCACGCTTCCCGAAGGGAGAAAGGCGGACAGGTATCCGGTAA | |
| | GCGGCAGGGTCGGAACAGGAGAGCGCACGAGGGAGCTTCCAGGGGGAAACGCC | |
| | TGGTATCTTTATAGTCCTGTCGGGTTTCGCCACCTCTGACTTGAGCGTCGATT | |
| | TTTGTGATGCTCGTCAGGGGGGCGGAGCCTATGGAAAAACGCCAGCAACGCGG | |
| | CCTTTTTACGGTTCCTGGCCTTTTGCTGGCCTTTTGCTCACATGTTCTTTCCT | |
| | GCGTTATCCCCTGATTCTGTGGATAACCGTATTACCGCCTTTGAGTGAGCTGA | |
| | TACCGCTCGCCGCAGCCGAACGACCGAGCGCAGCGAGTCAGTGAGCGAGG | |
| MB9997 | AAGCGGAAGAGCGCCCAATACGCAAACCGCCTCTCCCCGCGCGTTGGCCGATT | 12 |
| | CATTAATGCAGCTGGCACGACAGGTTTCCCGACTGGAAAGCGGGCAGTGAGCG | |
| | CAACGCAATTAATGTGAGTTAGCTCACTCATTAGGCACCCCAGGCTTTACACT | |
| | TTATGCTTCCGGCTCGTATGTTGTGTGGAATTGTGAGCGGATAACAATTTCAC | |
| | ACAGGAAACAGCTATGACCATGATTACGCCAAGCTTGGTACCAGAGACGGGTT | |
| | GGCGGCGTATTTGTGTCCCAAAAAACAGCCCCAATTGCCCCAATTGACCCCAA | |
| | ATTGACCCAGTAGCGGGCCCAACCCCGGCGAGAGCCCCCTTCACCCCACATAT | |
| | CAAACCTCCCCCGGTTCCCACACTTGCCGTTAAGGGCGTAGGGTACTGCAGTC | |
| | TGGAATCTACGCTTGTTCAGACTTTGTACTAGTTTCTTTGTCTGGCCATCCGG | |
| | GTAACCCATGCCGGACGCAAAATAGACTACTGAAAATTTTTTTGCTTTGTGGT | |
| | TGGGACTTTAGCCAAGGGTATAAAAGACCACCGTCCCCGAATTACCTTTCCTC | |
| | TTCTTTTCTCTCTCTCCTTGTCAACTCACACCCGAAATCGTTAAGCATTTCCT | |
| | TCTGAGTATAAGAATCATTCGCTAGCCCACAAAAATGGTTAACGTTGCTTACG | |
| | AGTGGCTTCGAGACTGCTGGCCCCCTAAGCCTGAGTTCCTCCCTAAGGATTAC | |
| | CCTGACCTCACCGGAAAGACCATCCTTATCACTGGTGCCAACACTGGTATTGG | |
| | TTTTGAGTCCGCTAAGGCTCTCCTCAAGAAGAACGCTACCGTTGTTTTTGCCA | |
| | ACCGATCCGAGGAGAAGACTAAGATCGCTATTGCCAAGATTCAGGCTGAGCTT | |
| | GGTGGTGATTCTTCTCAGCGATCTATTTTCGTCAAGACTGACCTTTCCGATCT | |
| | CTCTTCCATTAAGGGCACTGCTGAGGAGCTTAAGTCTAAGGGTATCACTAAGC | |
| | TCCACTACACCATTCTTAACGCTGGTGTTATGCAGCCTCCCAAGGGCTCCAAG | |
| | ACTAAGCAGGGTTTTGAGCTTCAGATTGGTACTAACGTTCTTGGCCACCAGCT | |
| | CCTCCAGAAGTTTCTCACTCCCCTTGTTCTCAACGCTGTTTCTTCTGATTTCA | |
| | CCCCTCGAGTTGTTTGGCTCGCCTCTGCTGCTCACCTTTCTTCCCCTCCCAAC | |
| | GGTGGTATTGATTGGGATTCCTTCCGAAACGCTGATTGGGCTGGTACTGTCTC | |
| | TGCTTACGGTCAGTCTAAGACTGGTAACATTTACCAGGCTTACATCTACGCTC | |
| | AGCAGCACAAGGATGTCATCTCTGTCTCTGCTCACCCCGGATACCTTGCTTCT | |
| | GATCTCACTCGAGCTTACAAGGGATACCTTCAGTACCTTATGAAGCTCCTTAT | |
| | GTCTCCTCCCGTTTACGGTTCTTACACTGAGCTTTTCGCTGCTCTTTCTCCCA | |
| | AGGTTACTCTCAAGGAGTCTGGTCGATACATTGGTCCTTGGGGTCAGTTCCGA | |
| | GAGCTTCGAGATGACGTTCAGGAGGGCCTTACCGATGGTACTGCTCAGAAGCT | |
| | CTGGGATTGGGCTGAGGCTGAGATTCGACCTTACACTTAAACGCGTGCAATTA | |
| | ACAGATAGTTTGCCGGTGATAATTCTCTTAACCTCCCACACTCCTTTGACATA | |
| | ACGATTTATGTAACGAAACTGAAATTTGACCAGATATTGTTGTAAATAGAAAA | |
| | TCTGGCTTGTAGGTGGCAAACTAGTGGTGTGTTCTGTGGAGCATTCTCACTTT | |
| | TGGTAAACGACATTGCTTCAAGTGCAGCGGAATCAAAAAGTATAAAGTGGGCA | |
| | GCGAGTATACCTGTACAGACTGTAGGCGATAACTCAATCCAATTACCCCCCAC | |
| | AACATGACTGGCCAAACTGATCTCAAGACTTTATTGAAATCAGCAACACCGAT | |
| | TCTCAATGAAGGCACATACTTCTTCTGCAACATTCACTTGACGCCTAAAGTTG | |
| | GTGAGAAATGGACCGACAAGACATATTCTGCTATCCACGGACTGTTGCCTGTG | |
| | TCGGTGGCTACAATACGTGAGTCAGAAGGGCTGACGGTGGTGGTTCCCAAGGA | |
| | AAAGGTCGACGAGTATCTGTCTGACTCGTCATTGCCGCCTTTGGAGTACGACT | |
| | CCAACTATGAGTGTGCTTGGATCACTTTGACGATACATTCTTCGTTGGAGGCT | |
| | GTGGGTCTGACAGCTGCGTTTTCGGCGCGGTTGGCCGACAACAATATCAGCTG | |
| | CAACGTCATTGCTGGCTTTCATCATGATCACATTTTTGTCGGCAAAGGCGACG | |
| | CCCAGAGAGCCATTGACGTTCTTTCTAATTTGGACCGATAGCCGTATAGTCCA | |
| | GTCTATCTATAAGTTCAACTAACTCGTAACTATTACCATAACATATACTTCAC | |
| | TGCCCCAGATAAGGTTCCGATAAAAAGTTCTGCAGACTAAATTTATTTCAGTC | |
| | TCCTCTTCACCACCAAAATGCCCTCCTACGAAGCTCGAGCTAACGTCCACAAG | |
| | TCCGCCTTTGCCGCTCGAGTGCTCAAGCTCGTGGCAGCCAAGAAAACCAACCT | |
| | GTGTGCTTCTCTGGATGTTACCACCACCAAGGAGCTCATTGAGCTTGCCGATA | |
| | AGGTCGGACCTTATGTGTGCATGATCAAGACCCATATCGACATCATTGACGAC | |
| | TTCACCTACGCCGGCACTGTGCTCCCCCTCAAGGAACTTGCTCTTAAGCACGG | |
| | TTTCTTCCTGTTCGAGGACAGAAAGTTCGCAGATATTGGCAACACTGTCAAGC | |
| | ACCAGTACAAGAACGGTGTCTACCGAATCGCCGAGTGGTCCGATATCACCAAC | |
| | GCCCACGGTGTACCCGGAACCGGAATCATTGCTGGCCTGCGAGCTGGTGCCGA | |
| | GGAAACTGTCTCTGAACAGAAGAAGGAGGACGTCTCTGACTACGAGAACTCCC | |
| | AGTACAAGGAGTTCCTGGTCCCCTCTCCCAACGAGAAGCTGGCCAGAGGTCTG | |
| | CTCATGCTGGCCGAGCTGTCTTGCAAGGGCTCTCTGGCCACTGGCGAGTACTC | |
| | CAAGCAGACCATTGAGCTTGCCCGATCCGACCCCGAGTTTGTGGTTGGCTTCA | |
| | TTGCCCAGAACCGACCTAAGGGCGACTCTGAGGACTGGCTTATTCTGACCCCC | |
| | GGGGTGGGTCTTGACGACAAGGGAGACGCTCTCGGACAGCAGTACCGAACTGT | |
| | TGAGGATGTCATGTCTACCGGAACGGATATCATAATTGTCGGCCGAGGTCTGT | |
| | ACGGCCAGAACCGAGATCCTATTGAGGAGGCCAAGCGATACCAGAAGGCTGGC | |
| | TGGGAGGCTTACCAGAAGATTAACTGTTAGAGGTTAGACTATGGATATGTAAT | |
| | TTAACTGTGTATATAGAGAGCGTGCAAGTATGGAGCGCTTGTTCAGCTTGTAT | |
| | GATGGTCAGACGACCTGTCTGATCGAGTATGTATGATACTGCACAACCTGTGT | |
| | ATCCGCATGATCTGTCCAATGGGGCATGTTGTTGTGTTTCTCGATACGGAGAT | |
| | GCTGGGTACAAGTAGCTAATACGATTGAACTACTTATACTTATATGAGGCTTG | |
| | AAGAAAGCTGACTTGTGTATGACTTATTCTCAACTACATCCCCAGTCACAATA | |
| | CCACCACTGCACTACCACTACACCTCGAGCATGCATCTAGTGGTGTGTTCTGT | |
| | GGAGCATTCTCACTTTTGGTAAACGACATTGCTTCAAGTGCAGCGGAATCAAA | |
| | AAGTATAAAGTGGGCAGCGAGTATACCTGTACAGACTGTAGGCGATAACTCAA | |
| | TCCAATTACCCCCCACAACATGACTGGCCAAACTGATCTCAAGACTTTATTGA | |
| | AATCAGCAACACCGATTCTCAATGAAGGCACATACTTCTTCTGCAACATTCAC | |
| | TTGACGCCTAAAGTTGGTGAGAAATGGACCGACAAGACATATTCTGCTATCCA | |
| | CGGACTGTTGCCTGTGTCGGTGGCTACAATACGTGAGTCAGAAGGGCTGACGG | |
| | TGGTGGTTCCCAAGGAAAAGGTCGACGAGTATCTGTCTGACTCGTCATTGCCG | |
| | CCTTTGGAGTACGACTCCAACTATGAGTGTGCTTGGATCACTTTGACGATACA | |
| | TTCTTCGTTGGAGGCTGTGGGTCTGACAGCTGCGTTTTCGGCGCGGTTGGCCG | |
| | ACAACAATATCAGCTGCAACGTCATTGCTGGCTTTCATCATGATCACATTTTT | |
| | GTCGGCAAAGGCGACGCCCAGAGAGCCATTGACGTTCTTTCTAATTTGGACCG | |
| | ATAGCCGTATAGTCCAGTCTATCTATAAGTTCAACTAACTCGTAACTATTACC | |
| | ATAACATATACTTCACTGCCCCAGATAAGGTTCCGATAAAAAGTTCTGCAGAC | |
| | TAAATTTATTTCAGTCTCCTCTTCACCACCAAAATGCCCTCCTACGAAGCTCG | |
| | AGCTAACGTCCACAAGTCCGCCTTTGCCGCTCGAGTGCTCAAGCTCGTGGCAG | |
| | CCAAGAAAACCAACCTGTGTGCTTCTCTGGATGTTACCACCACCAAGGGGCCG | |
| | CTCGAGCATGCATCTAGAGGGCCCAATTCGCCCTATAGTGAGTCGTATTACAA | |
| | TTCACTGGCCGTCGTTTTACAACGTCGTGACTGGGAAAACCCTGGCGTTACCC | |
| | AACTTAATCGCCTTGCAGCACATCCCCCTTTCGCCAGCTGGCGTAATAGCGAA | |
| | GAGGCCCGCACCGATCGCCCTTCCCAACAGTTGCGCAGCCTGAATGGCGAATG | |
| | GACGCGCCCTGTAGCGGCGCATTAAGCGCGGCGGGTGTGGTGGTTACGCGCAG | |
| | CGTGACCGCTACACTTGCCAGCGCCCTAGCGCCCGCTCCTTTCGCTTTCTTCC | |
| | CTTCCTTTCTCGCCACGTTCGCCGGCTTTCCCCGTCAAGCTCTAAATCGGGGG | |
| | CTCCCTTTAGGGTTCCGATTTAGTGCTTTACGGCACCTCGACCCCAAAAAACT | |
| | TGATTAGGGTGATGGTTCACGTAGTGGGCCATCGCCCTGATAGACGGTTTTTC | |
| | GCCCTTTGACGTTGGAGTCCACGTTCTTTAATAGTGGACTCTTGTTCCAAACT | |
| | GGAACAACACTCAACCCTATCTCGGTCTATTCTTTTGATTTATAAGGGATTTT | |
| | GCCGATTTCGGCCTATTGGTTAAAAAATGAGCTGATTTAACAAAAATTTAACG | |
| | CGAATTTTAACAAAATTCAGGGCGCAAGGGCTGCTAAAGGAAGCGGAACACGT | |
| | AGAAAGCCAGTCCGCAGAAACGGTGCTGACCCCGGATGAATGTCAGCTACTGG | |
| | GCTATCTGGACAAGGGAAAACGCAAGCGCAAAGAGAAAGCAGGTAGCTTGCAG | |
| | TGGGCTTACATGGCGATAGCTAGACTGGGCGGTTTTATGGACAGCAAGCGAAC | |
| | CGGAATTGCCAGCTGGGGCGCCCTCTGGTAAGGTTGGGAAGCCCTGCAAAGTA | |
| | AACTGGATGGCTTTCTTGCCGCCAAGGATCTGATGGCGCAGGGGATCAAGATC | |
| | TGATCAAGAGACAGGATGAGGATCGTTTCGCATGATTGAACAAGATGGATTGC | |
| | ACGCAGGTTCTCCGGCCGCTTGGGTGGAGAGGCTATTCGGCTATGACTGGGCA | |
| | CAACAGACAATCGGCTGCTCTGATGCCGCCGTGTTCCGGCTGTCAGCGCAGGG | |
| | GCGCCCGGTTCTTTTTGTCAAGACCGACCTGTCCGGTGCCCTGAATGAACTGC | |
| | AGGACGAGGCAGCGCGGCTATCGTGGCTGGCCACGACGGGCGTTCCTTGCGCA | |
| | GCTGTGCTCGACGTTGTCACTGAAGCGGGAAGGGACTGGCTGCTATTGGGCGA | |
| | AGTGCCGGGGCAGGATCTCCTGTCATCCCACCTTGCTCCTGCCGAGAAAGTAT | |
| | CCATCATGGCTGATGCAATGCGGCGGCTGCATACGCTTGATCCGGCTACCTGC | |
| | CCATTCGACCACCAAGCGAAACATCGCATCGAGCGAGCACGTACTCGGATGGA | |
| | AGCCGGTCTTGTCGATCAGGATGATCTGGACGAAGAGCATCAGGGGCTCGCGC | |
| | CAGCCGAACTGTTCGCCAGGCTCAAGGCGCGCATGCCCGACGGCGAGGATCTC | |
| | GTCGTGACCCATGGCGATGCCTGCTTGCCGAATATCATGGTGGAAAATGGCCG | |
| | CTTTTCTGGATTCATCGACTGTGGCCGGCTGGGTGTGGCGGACCGCTATCAGG | |
| | ACATAGCGTTGGCTACCCGTGATATTGCTGAAGAGCTTGGCGGCGAATGGGCT | |
| | GACCGCTTCCTCGTGCTTTACGGTATCGCCGCTCCCGATTCGCAGCGCATCGC | |
| | CTTCTATCGCCTTCTTGACGAGTTCTTCTGAATTGAAAAAGGAAGAGTATGAG | |
| | TATTCAACATTTCCGTGTCGCCCTTATTCCCTTTTTTGCGGCATTTTGCCTTC | |
| | CTGTTTTTGCTCACCCAGAAACGCTGGTGAAAGTAAAAGATGCTGAAGATCAG | |
| | TTGGGTGCACGAGTGGGTTACATCGAACTGGATCTCAACAGCGGTAAGATCCT | |
| | TGAGAGTTTTCGCCCCGAAGAACGTTTTCCAATGATGAGCACTTTTAAAGTTC | |
| | TGCTATGTGGCGCGGTATTATCCCGTATTGACGCCGGGCAAGAGCAACTCGGT | |
| | CGCCGCATACACTATTCTCAGAATGACTTGGTTGAGTACTCACCAGTCACAGA | |
| | AAAGCATCTTACGGATGGCATGACAGTAAGAGAATTATGCAGTGCTGCCATAA | |
| | CCATGAGTGATAACACTGCGGCCAACTTACTTCTGACAACGATCGGAGGACCG | |
| | AAGGAGCTAACCGCTTTTTTGCACAACATGGGGGATCATGTAACTCGCCTTGA | |
| | TCGTTGGGAACCGGAGCTGAATGAAGCCATACCAAACGACGAGCGTGACACCA | |
| | CGATGCCTGTAGCAATGGCAACAACGTTGCGCAAACTATTAACTGGCGAACTA | |
| | CTTACTCTAGCTTCCCGGCAACAATTAATAGACTGGATGGAGGCGGATAAAGT | |
| | TGCAGGACCACTTCTGCGCTCGGCCCTTCCGGCTGGCTGGTTTATTGCTGATA | |
| | AATCTGGAGCCGGTGAGCGTGGGTCTCGCGGTATCATTGCAGCACTGGGGCCA | |
| | GATGGTAAGCCCTCCCGTATCGTAGTTATCTACACGACGGGGAGTCAGGCAAC | |
| | TATGGATGAACGAAATAGACAGATCGCTGAGATAGGTGCCTCACTGATTAAGC | |
| | ATTGGTAACTGTCAGACCAAGTTTACTCATATATACTTTAGATTGATTTAAAA | |
| | CTTCATTTTTAATTTAAAAGGATCTAGGTGAAGATCCTTTTTGATAATCTCAT | |
| | GACCAAAATCCCTTAACGTGAGTTTTCGTTCCACTGAGCGTCAGACCCCGTAG | |
| | AAAAGATCAAAGGATCTTCTTGAGATCCTTTTTTTCTGCGCGTAATCTGCTGC | |
| | TTGCAAACAAAAAAACCACCGCTACCAGCGGTGGTTTGTTTGCCGGATCAAGA | |
| | GCTACCAACTCTTTTTCCGAAGGTAACTGGCTTCAGCAGAGCGCAGATACCAA | |
| | ATACTGTTCTTCTAGTGTAGCCGTAGTTAGGCCACCACTTCAAGAACTCTGTA | |
| | GCACCGCCTACATACCTCGCTCTGCTAATCCTGTTACCAGTGGCTGCTGCCAG | |
| | TGGCGATAAGTCGTGTCTTACCGGGTTGGACTCAAGACGATAGTTACCGGATA | |
| | AGGCGCAGCGGTCGGGCTGAACGGGGGGTTCGTGCACACAGCCCAGCTTGGAG | |
| | CGAACGACCTACACCGAACTGAGATACCTACAGCGTGAGCTATGAGAAAGCGC | |
| | CACGCTTCCCGAAGGGAGAAAGGCGGACAGGTATCCGGTAAGCGGCAGGGTCG | |
| | GAACAGGAGAGCGCACGAGGGAGCTTCCAGGGGGAAACGCCTGGTATCTTTAT | |
| | AGTCCTGTCGGGTTTCGCCACCTCTGACTTGAGCGTCGATTTTTGTGATGCTC | |
| | GTCAGGGGGGCGGAGCCTATGGAAAAACGCCAGCAACGCGGCCTTTTTACGGT | |
| | TCCTGGCCTTTTGCTGGCCTTTTGCTCACATGTTCTTTCCTGCGTTATCCCCT | |
| | GATTCTGTGGATAACCGTATTACCGCCTTTGAGTGAGCTGATACCGCTCGCCG | |
| | CAGCCGAACGACCGAGCGCAGCGAGTCAGTGAGCGAGG | |

The invention claimed is:

1. A retinol-producing host cell expressing a retinol dehydrogenase 22 (RDH22) homolog with at least about 30% identity to a polynucleotide according to SEQ ID NO: 1 or 4, wherein the host cell has been transformed with said polynucleotide encoding said RDH22 homolog and wherein the percentage of dihydroretinoids in a mix of retinoids produced using said host cell under suitable culture conditions is less than about 10% based on total retinoids.

2. The retinol-producing host cell according to claim 1, wherein said process furthermore comprises acetylation of retinol into retinyl acetate.

3. The retinol-producing host cell according to claim 2, wherein the percentage of retinyl acetate in said mix is at least about 80% based on total retinoids.

4. The retinol producing host cell according to claim 1, wherein the percentage of retinal in said mix is less than about 8% based on total retinoids.

5. The retinol-producing host cell according to claim 4, wherein the percentage of retinal is reduced to less than about 1% based on total retinoids.

6. The retinol producing host cell according to claim 1, wherein said mix of retinoids comprises trans and cis-isomers and wherein the percentage of trans-isomers is at least about 80% based on total cis and trans-isomers in said mix.

7. The retinol-producing host cell according to claim 1, wherein the RDH22 homolog is obtained from Yarrowia or Wickerhamomyces.

8. The retinol-producing host cell according to claim 1, furthermore expressing a retinol dehydrogenase 12 (RDH12) homolog.

9. Process for the production of a retinoid-mix comprising cultivation of a retinol-producing host cell according to claim 1 under suitable culture conditions.

10. Process according to claim 9, wherein said retinoid mix comprises dihydroretinal, dihydroretinol and dihydroretinyl acetate, retinol, retinal, retinyl acetate, and wherein the percentage of dihydroretinoids based on total retinoids is less than 10%.

11. Process according to claim 9, wherein the host cell is cultivated in the presence of a lipophilic solvent.

12. Process according to claim 11, wherein the retinoids are accumulated in the lipophilic solvent and optionally extracted from the lipophilic solvent.

13. Process according to claim 11, wherein the lipophilic solvent is selected from isoparaffins including mixtures of alkanes, cycloparaffin, isoalkanes, cycloalkanes, or dodecanes.

14. Process according to claim 9, wherein the host cell is a fungal host cell.


SEQUENCE LISTING (12 sequences)

SEQ ID NO: 1 - protein, 323 aa, Yarrowia lipolytica
MTRSKFTEYKQVLRNSCVGKPQYTEKDYPDLDGKNFLVTGATGGVGLEATKLLLEKKSHVIMVGRSKTKSQSTLDELQKTYSHGTFDFVEADLSDLTTVERAGEYIRSKYTTLDGAILNAGVMAPPYSLTPQGHESQWGINVVAHFLLSKYISPALISAAQTAPKDTVRLVWVSSSVVAMSPYEGGIKFDDINHSKVKNPSPWTLYSQSKIGDAYLAYLWSKHHPDSGVLSVSLDPGNLASDLSRHTSWLSSIKNYVLYPPKYGAYTELSALLNPSVKNNEHLIPWGIEGHLRQDVDDGRRGKDGEELWQGLNKDVEGFFKEE

SEQ ID NO: 2 - DNA, 972 bp, Yarrowia lipolytica
  source 1..972: mol_type = genomic DNA
atgactcgatccaagttcaccgaatacaaacaagtgctgcgaaacagctgcgtgggcaagccccaatacaccgagaaggattaccctgatctcgacggaaagaactttcttgtcactggagccactggaggagttggtctggaggccaccaagctgctgctagagaagaagagccatgtaatcatggttggccgatccaaaacaaagtctcagtctacccttgacgagctccagaagacatactcccacggtacctttgatttcgtcgaggctgatctgtctgacctgaccactgtggagcgagccggagagtacattcgaagtaagtacaccactttggacggtgccattctcaacgctggagtcatggctcccccctatagcctcactccccagggccacgagtctcaatggggtatcaatgtggttgcccattttctgctgtccaagtatatctctcctgctctgatctcggccgcccagacagcccccaaggacaccgtgcgacttgtctgggtgtcttcttctgtcgttgccatgagtccttatgaaggaggtatcaagtttgacgatatcaaccactctaaggtcaagaacccttctccctggaccctttactcccagtccaagattggagatgcttacctggcttacctgtggtcgaaacaccatcccgattctggcgtcctgtccgtctctctggaccccggtaacctggcctctgatctgtccagacatacttcttggctctccagcattaagaactacgtgctgtatcctcccaagtatggcgcctacaccgagctgtctgctctgctgaacccctccgtcaagaataacgagcatctcattccctggggtattgagggacaccttagacaggatgtcgacgatggacgacgtggaaaggacggagaagagctctggcagggtctcaacaaggatgtcgagggcttctttaaggaggagtaa

SEQ ID NO: 3 - DNA, 972 bp, synthetic construct
  misc_feature 1..972: note = codon-optimized for expression in Y. lipolytica
  source 1..972: mol_type = other DNA
atgacccgatccaagttcaccgagtacaagcaggttctccgaaactcttgtgttggcaagccccagtacactgagaaggattaccccgatctcgacggaaagaactttctcgtcactggtgctactggtggagttggtctggaggccactaagctgctccttgagaagaagtctcacgttattatggttggccgatctaagactaagtctcagtctacccttgacgagcttcagaagacttactctcacggtactttcgatttcgttgaggctgatctctctgaccttaccactgttgagcgagccggagagtacattcgatctaagtacactacccttgacggtgccattcttaacgctggagtcatggctcccccctactctctcactccccagggtcacgagtctcagtggggtattaacgttgttgctcactttctcctgtctaagtacatctctcctgctctgatttctgctgctcagactgcccccaaggacaccgttcgacttgtttgggtgtcttcttctgtcgttgccatgtctccttacgagggaggtattaagtttgacgatattaaccactctaaggtcaagaacccttctccttggaccctttactctcagtctaagattggtgatgcttacctggcttacctttggtctaagcaccaccccgattctggagtcctttctgtctctctggaccccggtaacctggcttctgatctctctcgacacacttcttggctttcttctattaagaactacgttctttaccctcccaagtacggtgcctacactgagctgtctgctcttctcaacccttccgttaagaacaacgagcaccttattccctggggtattgagggacaccttcgacaggatgtcgacgatggtcgacgaggaaaggacggagaggagctctggcagggtcttaacaaggatgtcgagggtttctttaaggaggagtaa

SEQ ID NO: 4 - protein, 319 aa, Wickerhamomyces anomalus
MVNVAYEWLRDCWPPKPEFLPKDYPDLTGKTILITGANTGIGFESAKALLKKNATVVFANRSEEKTKIAIAKIQAELGGDSSQRSIFVKTDLSDLSSIKGTAEELKSKGITKLHYTILNAGVMQPPKGSKTKQGFELQIGTNVLGHQLLQKFLTPLVLNAVSSDFTPRVVWLASAAHLSSPPNGGIDWDSFRNADWAGTVSAYGQSKTGNIYQAYIYAQQHKDVISVSAHPGYLASDLTRAYKGYLQYLMKLLMSPPVYGSYTELFAALSPKVTLKESGRYIGPWGQFRELRDDVQEGLTDGTAQKLWDWAEAEIRPYT

SEQ ID NO: 5 - DNA, 960 bp, Wickerhamomyces anomalus
  source 1..960: mol_type = genomic DNA
atggttaacgttgcttatgaatggctaagagactgctggcctcctaaacctgaattcttaccaaaagattatccagatttaacaggaaagacaatcttaatcactggtgccaatacgggtattgggtttgaaagtgctaaagccttgttgaagaaaaacgcaacagttgtatttgcaaatagaagcgaagagaagacaaaaatcgctattgccaaaattcaagctgaattaggtggtgattcatctcaaagatcaattttcgtcaagactgatttaagtgatttatcatcgatcaagggcacagcggaagaattgaaatcaaaaggtatcacaaaattacattacaccatattgaatgctggtgttatgcaacctccaaaaggttccaagacaaaacaaggatttgaattacaaattggtacaaatgttttaggtcatcaattattacaaaaatttctaacaccattagttttgaatgcagtttcatcagatttcacaccaagagttgtttggttagcctcagctgcacatctttcatccccaccaaatggtggtattgattgggattccttccgtaatgctgattgggctggaactgtcagtgcttatggtcaatccaagactggtaatatttatcaagcttatatctatgctcagcaacataaagatgtcatcagtgtctctgctcatccagggtatttggcttctgatttaacaagagcttataaaggttatcttcaatatttgatgaaactcttgatgtctcctccagtttatggttcttatactgaattattcgctgctctcagtccaaaagttacattgaaggaaagtggtagatatattggtccttggggtcaattcagagaattgagagatgatgttcaagaaggcttaactgatggaactgctcaaaaattatgggattgggctgaagctgaaattagaccatacacttag

SEQ ID NO: 6 - DNA, 960 bp, synthetic construct
  misc_feature 1..960: note = codon-optimized for expression in Y. lipolytica
  source 1..960: mol_type = other DNA
atggttaacgttgcttacgagtggcttcgagactgctggccccctaagcctgagttcctccctaaggattaccctgacctcaccggaaagaccatccttatcactggtgccaacactggtattggttttgagtccgctaaggctctcctcaagaagaacgctaccgttgtttttgccaaccgatccgaggagaagactaagatcgctattgccaagattcaggctgagcttggtggtgattcttctcagcgatctattttcgtcaagactgacctttccgatctctcttccattaagggcactgctgaggagcttaagtctaagggtatcactaagctccactacaccattcttaacgctggtgttatgcagcctcccaagggctccaagactaagcagggttttgagcttcagattggtactaacgttcttggccaccagctcctccagaagtttctcactccccttgttctcaacgctgtttcttctgatttcacccctcgagttgtttggctcgcctctgctgctcacctttcttcccctcccaacggtggtattgattgggattccttccgaaacgctgattgggctggtactgtctctgcttacggtcagtctaagactggtaacatttaccaggcttacatctacgctcagcagcacaaggatgtcatctctgtctctgctcaccccggataccttgcttctgatctcactcgagcttacaagggataccttcagtaccttatgaagctccttatgtctcctcccgtttacggttcttacactgagcttttcgctgctctttctcccaaggttactctcaaggagtctggtcgatacattggtccttggggtcagttccgagagcttcgagatgacgttcaggagggccttaccgatggtactgctcagaagctctgggattgggctgaggctgagattcgaccttacacttaa

SEQ ID NO: 7 - DNA, 11606 bp, synthetic construct
  misc_feature 1..11606: note = expression plasmid
  source 1..11606: mol_type = other DNA
cgcgtggatcgccggtgcgttgacgttggtgacctccagccagaggtgcccggcgccccgctcgccggcgaactccgtcgcgagccccatcaacgcgcgcccgaccccgtgcccccggtgctccggggcgacctcgatgtcctcgacggtcagccggcggttccacgccgagtacgagatgaccacgaagcccgccaggtcgccgtcgtccccgtacgcgacgaacgtccgggagtccgggtcgccgtcctccccgtcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctagaggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatggccgccagtgtgctggaattgaatatttaccgttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgttaggggcagggcatgctcatgtagagcgcctgccgctcgccgtccgaggcggtgccgtcgtacagggcggtgtccaggccgcagagggtgaaccccatccgccggta

SEQ ID NO: 8 - DNA, 14006 bp, synthetic construct
  misc_feature 1..14006: note = expression plasmid
  source 1..14006: mol_type = other DNA
gcggagcctaggccggccagcgtggcgcgtggccatattggccagcagcttactacagctcagccaaagtggatcatgatggacggaaatatcggactggaggccaaaaaggaggtgctcaaatacgcacgggacaagtctgcacaggtggcattcgaacccacgtctgtccccaaagccgctgctctttccgagctcaatctgcccgtttaccccaacaactccattgccttagccacccccaacactgcagagctcgaggccatgttcgaggcattccacgagaagggccgatttgacgttgacgactggtttccagtcatagacggtcttgctctcggagctgactttagaaacggagccaccatgttgtcgcaccaacatcgtggactcaaagctattctcgaacaaggaactctggcccaggcaatccacatgctaccatacatcccaactttgatcatcaagggcggagccaacggcgtcgtcgtcttccaactcattgatgatatcgagtctgccattcattcacagcgttctgcttccaataaaacgcctggcctgttccagaagggcaacgctgcgagtggaaacaccaaggtcggcgtctacatgcagtactttgagcccgaagaagtgggcagtcagtcgattgtaagcgtgactggagctggagacactctgtttggaaccctggccatggagattgtcaaggacgagtcctggttgaacgatatgggagacaagaagagtgcagttgtttctcgagccatgaacaatgctgtgaagactattcagagtaaggacgctgtctgcaagagcatcctttaagtgatttgccatgctttctcttcttccacgatgtaaatacttattttacacactactgtgcagtagcaaatacagaacaagagttgtcgcctattgacagtacagtacgagtagtgtatgtacagtagttatacaatatctatgtgaaattcgtcggcagctttcggctgatgaactacgagttcttcgtaataaatcatcaacgtaataagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgggtgatctcgatgcccgaggaacctctgctcaccccgagctctctgagcgaccttctattatgccttctatgtctgatattcaggacccttctggtgatgacaaggctactccccgaggatctgctgctggtctgccccagcttgagcttgctggacacgcccgacgacttggccaccttgagaacttctttgctgtccaggctcgacagcagatttactcttcttttgctgttttttgtgagtttgacactgcttgttctctcgctcagcttgcttctgctgtgcgaaacgtttgtctttctaacccccttctccttcacactgttgagcctaagcaccctgacatcgctggattctaccactctgacgagtacctttcccgaccttggcctcagcacgattacatgcgagttcttcgagaggttcacgtcgctgacgttgttatgaacggacagaaggagcacgctcacgttgttcgagatgctgttgacgtttttcaggctcacggaaaccaggttacttctgagctccttgagcttatgactcagattgagattcctcacgcttctcagactcgaccctcttggcgacttctctgttttccccacggagaggctaaccgatggcgaacctttgcttttgtttctaaccactgttcttctgatggtctttctggtcttaacttctttcgagatctccagaaggagcttgctcacggccccacctctggtgctcctggtgcccccggagcttccggagttattttcgattacgctcaggacgctgctaccctgcccaagctgccccctcccattgatcagaagctcgattaccgaccttctaagaaggctcttctcggccttctcgctggcaagttcgttcgagagaagctcggttacgtttctgctgctcctcccactacccctacctctgaccttgctcaccctgagggtcaccagtactactgttaccttgttaacgttcccacttcttctgttgcccacattaagactcaggtgcgagagaacgttcctcacaagtgtactctcactccctttctccaggcttgttggcttgtttctctgttcaagtacggtcgagttttttctggttcttggcttgagcgatacaccgatgttcttgttgctatgaacactcgacagcttctccccgaggaccttgagcttcagcgacagtaccgatacggttctaacattggagctgttcgatacaactaccctattgctccccttgacgttcgagataacgatcagaagttctggtcccttgttgagtcttaccgacttgccctttctgatgcccgagataagaacgattacctttacgctcttggtgctcttatgctccctgagatttacgagaagaagaacgttgatgctgttgttaacgataccattcttaaccagcgacgacagggaacccttctttctaacgttggttacgttcgagatgagcagcccactgcttttgctattaagaaccacgttttttctcagggagttggagctaaccgaaacgcttttgttcttaacatttgtgctaccgatcagggtggtcttaacatcgctatttctattgctaagggaacccttgcttctcgacaggagggacaggagctttgtgatatttttaagtctactctccttcgattttaaacgcgtctatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatctgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtaccattccaaccgtctcagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcgtagaccccggtgtatataaaggggtggagaggacggattattagcaccaacacacacacttatactacatgctagccacaaaaatgctctctttcttctggcgaaacggtatcgagactcccgagcccctcaaggctgacgtttccggctctatccctccctggcttcagggaacccttctccgaaacggtcctggtctgttctccgttggcaacacttcctacaagcactggttcgatggtatggctctcattcactccttcacctttaaggatggtgaggttttttaccgatctaagtacctgaagtctgagacttacaagaagaacatcgctgccgaccgaatcgttgtgtctgagttcggaaccatggtgtaccccgatccctgcaagaacattttctcccgagccttctcttacatgatgaacgccattcctgactttaccgataacaacctcattaacatcattaagtacggtgaggattactacgcctcctctgaggtcaactacatcaaccagattgaccccctgacccttgagactctcggacgaactaactaccgaaaccacattgccatcaaccttgccactgctcaccctcactacgacgaggagggtaacacttacaacatgggcactgctattatgaacctcggtcgacccaagtacgtgattttcaaggtgcccgccaacacctctgataaggagaacaagaagcctgccctctctgaggtggagcaggtttgctccattcccatccgaccctccctttacccttcttacttccactcttttggcatgactgagaactacatcatcttcgttgagcaggccttcaagctggacatcgtcaagctggctactgcttacttccgagatattaactggggatcttgccttaagttcgaccaggatgacattaacgtgttccacctggtcaacaagaagactggtaaggctgtgtccgtgaagtactacactgacccctttgttaccttccaccacatcaacgcttacgaggacgatggccacgtcgtcttcgatctcattacttacaaggactctaagctgtacgatatgttctacattcagaacatgaagcaggacgtcaagcgatttattgagactaacaaggacttcgctcagcccgtgtgccagcgatttgtccttcccgtcaacgttgataaggagactcctcaggacatcaaccttgtcaagctgcaggacaccactgccactgctgtcctgaaggaggacggctctgtctactgcacccctgacatcatttttaagggtcttgagctccctgctatcaactacaagtttaactctaagaagaaccgatacttctacggcacccgagtggagtggtccccttaccctaacaaggtcgctaaggtggacgttgttactcgaacccacaagatttggactgaggaggagtgttacccttctgagcctgtctttattgcctcccctgacgccgttgatgaggatgacggtgtgattctttcttctgtggtttctttcaacccccagcgaccccctttcctggttgtcctcgatgctaagtccttcaaggagattgctcgagctaccatcgatgcctctattcacatggaccttcacggccttttcatccacgacaagtctacctaagttttttgatcaatgatccaatggctttcacatacccccccacgcctataattaaaacacagagaaatataatctaacttaataaatattacggagaatctttcgagtgttcagcagaaatatagccattgtaacaaaagccggctatcgaccgctttatcgaagaatatttcccgccccccagtggccaaacgatatcggtcagaaggggcagctctaaacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacatgctagccacaaaaatgaccactaagtacacttccgttcacgagtctcccaacggccctggtgacgctcgacccaccgcttcccagattatcgacgattacaaccttgagggagagctttctggcaagactgttctcgtcaccggctgttcctctggtattggtgttgagactgcccgagctatttaccgaactggtgccaccctttacctcactgcccgagatgtcgataaggccaagaccgttcttcccgaccttgttgacacttcccgagtccactttctccaccttgaccttaactctctggagtctgttcgaggttttgctgagaacttcaagtctaagtccactcagcttcacattctcatcgagaacgctggcgtgatggcctgtcccgagggccgaaccgtcgatggttttgagactcagtttggtatcaaccaccttgctcactttctcctcttttacctcctcaaggatacccttctcaactcttctacccccgctttcaactcccgagttgtcatcctctcttcttgtgctcaccaggctggttccgttcaccttaacaacctgtctcttgagggtggatacgagccttggaagtcttacggccagtccaagactgccaacctttggactgcccgagagatcgagaagcgatttggtgcttccggtatccactcttgggctgttcaccccggttccatcgctactgagcttcagcgacacgtttccgacgagcttaagcagaagtgggctgacgataaggagggtgccaagctgtggaagtccaccgagcagggtgccgccaccactgtccttgctgctgtttcccctgagcttgagggtaagggcggtctttaccttgaggatacccaggttgccaagccccctgcccgaggaatgtttggtgttgctgactgggcttacgatgaggatggcccctctaagctctgggccaagtctcttgagctccttaagctccagtaaaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcacactattatcacattactacatccaaacccctacagggggaggagctctccaatcaaatatgtacattaactatctctcgtaaatcattgttataagaccgtccgtgactgtctaaatcggttcattcgttgtaacaaatcagtgtaacaactcgtggtacgcccggtttgcttcgctcacgctcccctagattttccgtctaggacacaacaagccctggtggatgacgttcaacgccttcagcgcgtctttttcctcaataacacacgagatgttaatttcgttggctccctgagaaatcatctcaatgttaatacctgcctgggccagagtagagaagaacatgcctgcacatccgaccatggccttcattctcgttccgatcagcgacacaatggtcatgcctcgcttcacatctactgtgccgtactttcgcagctcctccacagcctgcttgaggttggagtcgggagcatggaaggccatcgacacatggacctcagaagtggagatgagatcgacgacaagtttctgctggtcgagagtagcaaagatcttgttcaagaagccatgactcttggttcgcttattcgaatgaacgttgagaacggtgatattcgatttggtagtgacagctgtaggcttcttctcgtcatctacaggagcagacacctggggagtgaatgagccagaggacagagaagttagcgagtcggtggaagaagccagatcctggacattgtttttcgttttcaggttgcccgagccgtcagcagatgggtagatgatagttcctcctcccagggggttttccacgtttttgattcggataggaatatgggccttaatgacctgctccatggtgaaggggtggatgacttcagatccgtagtaggtcaactcagcggcctcctcgggggtaatgatgggcagcagacgggcagtggacacctttctgggatcggcagtgaagacaccatcgacttctttccagatctgaagctccttggcgtccagacccacagccaccagagcagcacacagatcggtgtatcctcgtccgatctggcttaggaggcctcccttgacggggccaaagaaaccggtcaacacgggcaccatgtttcccttgacaggagcagagtccgtgttaggagagccgggaagagtgatgatctcgcccagaacacgtccgaggtccgaatagaacttgggatcttcggcattggtggtggtaactgcgtgagacaggtcaaagtaccgtgcgttaacaccagcgtcccgcataactgcagtcatgtacatgcaactgagcttctccccaatggccatgatggagtcgagtgttcggggggagatctcagaaatgatttcagcagcagccaggattcgcaacagctggtcgcactcgccgttgatgtcggcattgaggttttcgagcagttcggggttcttgacgtcacgcttagcagctgcaagatggtcttctcggatagactcaatgatggggttgtaggcgtctgatccgagaagagcagagtcagcagcagcaataagacgggtggtggttccctcggccttggtggcctctagatggcctccttggccccattccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgag

SEQ ID NO: 9 - DNA, 21335 bp, synthetic construct
  misc_feature 1..21335: note = plasmid with Cas9; LIP8 targeting guide RNAs
  source 1..21335: mol_type = other DNA
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgacgagactgatgagtccgtgaggacgaaacgagtaagctcgtcgagggctgcaaaattcacgagttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgttggaccccgtcttcaattacacttcccaactgggaacacccctctttatcgacccattttaggtaatttaccctagcccattgtctccataaggaatattaccctaacccacagtccagggtgcccaggtccttctttggccaaattttaacttcggtcctatggcacagcggtagcgcgtgagattgcaaatcttaaggtcccgagttcgaatctcggtgggacctagttgaaaaatacctctaatgcgccgatggtttagtggtaaaatccatcgttgccatcgatgggcccccggttcgattccgggtcggcgcaggttgacgtacagcaggctgaacgaggatgttttagagctagaaatagcaagttaaaataaggctagtccgttatcaacttgaaaaagtggcaccgagtcggtgcttttttttttcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctagaaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgatggctccctccattcgaaagttctttgctggtggtgtgtgtcgaactaacgttcagcttcccggtaaggtggttgtcatcactggtgccaacaccggcattggcaaggagactgcccgagagctcgcttcccgaggagcccgagtttacattgcttgccgagatgttctgaagggcgagtctgctgcctctgagattcgagttgacactaagaactcccaggtgctcgtgcgaaagctcgacctttccgacactaagtctatccgagcctttgctgagggctttctcgctgaggagaagcagcttcacattctgattaacaacgctggagttatgatgtgtccttactctaagactgctgatggtttcgagactcacctcggagtcaaccacctgggccacttcctcctcacctacctgctcctggagcgactcaaggtgtctgcccctgcccgagtggttaacgtttcctccgtggctcaccacattggcaagattcccttccacgacctccagtccgagaagcgatactcccgaggttttgcttactgccactccaagctggccaacgttctctttacccgagagctggccaagcgactccagggaaccggcgtcaccacctacgccgttcaccccggtgtcgtccgatccgagctggtccgacactcctccctgctctgcctgctctggcgactcttctcccccttcgttaagaccgcccgagagggtgcccagacctccctgcactgcgccctggctgagggcctggagcccctgtctggcaagtacttctctgactgcaagcgaacctgggtgtctccccgagctcgaaacaacaagactgccgagcgactctggaacgtttcctgtgagcttctcggtattcgatgggagtagacgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcatctagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagg

SEQ ID NO: 10 - DNA, 8089 bp, synthetic construct
  misc_feature 1..8089: note = expression plasmid
  source 1..8089: mol_type = other DNA
aagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagccacaaaaatgatggctccctccattcgaaagttctttgctggtggtgtgtgtcgaactaacgttcagcttcccggtaaggtggttgtcatcactggtgccaacaccggcattggcaaggagactgcccgagagctcgcttcccgaggagcccgagtttacattgcttgccgagatgttctgaagggcgagtctgctgcctctgagattcgagttgacactaagaactcccaggtgctcgtgcgaaagctcgacctttccgacactaagtctatccgagcctttgctgagggctttctcgctgaggagaagcagcttcacattctgattaacaacgctggagttatgatgtgtccttactctaagactgctgatggtttcgagactcacctcggagtcaaccacctgggccacttcctcctcacctacctgctcctggagcgactcaaggtgtctgcccctgcccgagtggttaacgtttcctccgtggctcaccacattggcaagattcccttccacgacctccagtccgagaagcgatactcccgaggttttgcttactgccactccaagctggccaacgttctctttacccgagagctggccaagcgactccagggaaccggcgtcaccacctacgccgttcaccccggtgtcgtccgatccgagctggtccgacactcctccctgctctgcctgctctggcgactcttctcccccttcgttaagaccgcccgagagggtgcccagacctccctgcactgcgccctggctgagggcctggagcccctgtctggcaagtacttctctgactgcaagcgaacctgggtgtctccccgagctcgaaacaacaagactgccgagcgactctggaacgtttcctgtgagcttctcggtattcgatgggagtagacgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcatctagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagg

SEQ ID NO: 11 - DNA, 8159 bp, synthetic construct
  misc_feature 1..8159: note = expression plasmid
  source 1..8159: mol_type = other DNA
aagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagcccacaaaaatgacccgatccaagttcaccgagtacaagcaggttctccgaaactcttgtgttggcaagccccagtacactgagaaggattaccccgatctcgacggaaagaactttctcgtcactggtgctactggtggagttggtctggaggccactaagctgctccttgagaagaagtctcacgttattatggttggccgatctaagactaagtctcagtctacccttgacgagcttcagaagacttactctcacggtactttcgatttcgttgaggctgatctctctgaccttaccactgttgagcgagccggagagtacattcgatctaagtacactacccttgacggtgccattcttaacgctggagtcatggctcccccctactctctcactccccagggtcacgagtctcagtggggtattaacgttgttgctcactttctcctgtctaagtacatctctcctgctctgatttctgctgctcagactgcccccaaggacaccgttcgacttgtttgggtgtcttcttctgtcgttgccatgtctccttacgagggaggtattaagtttgacgatattaaccactctaaggtcaagaacccttctccttggaccctttactctcagtctaagattggtgatgcttacctggcttacctttggtctaagcaccaccccgattctggagtcctttctgtctctctggaccccggtaacctggcttctgatctctctcgacacacttcttggctttcttctattaagaactacgttctttaccctcccaagtacggtgcctacactgagctgtctgctcttctcaacccttccgttaagaacaacgagcaccttattccctggggtattgagggacaccttcgacaggatgtcgacgatggtcgacgaggaaaggacggagaggagctctggcagggtcttaacaaggatgtcgagggtttctttaaggaggagtaaacgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcatctagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagg

SEQ ID NO: 12 - DNA, 8147 bp, synthetic construct
  misc_feature 1..8147: note = expression plasmid
  source 1..8147: mol_type = other DNA
aagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccagagacgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcgctagcccacaaaaatggttaacgttgcttacgagtggcttcgagactgctggccccctaagcctgagttcctccctaaggattaccctgacctcaccggaaagaccatccttatcactggtgccaacactggtattggttttgagtccgctaaggctctcctcaagaagaacgctaccgttgtttttgccaaccgatccgaggagaagactaagatcgctattgccaagattcaggctgagcttggtggtgattcttctcagcgatctattttcgtcaagactgacctttccgatctctcttccattaagggcactgctgaggagcttaagtctaagggtatcactaagctccactacaccattcttaacgctggtgttatgcagcctcccaagggctccaagactaagcagggttttgagcttcagattggtactaacgttcttggccaccagctcctccagaagtttctcactccccttgttctcaacgctgtttcttctgatttcacccctcgagttgtttggctcgcctctgctgctcacctttcttcccctcccaacggtggtattgattgggattccttccgaaacgctgattgggctggtactgtctctgcttacggtcagtctaagactggtaacatttaccaggcttacatctacgctcagcagcacaaggatgtcatctctgtctctgctcaccccggataccttgcttctgatctcactcgagcttacaagggataccttcagtaccttatgaagctccttatgtctcctcccgtttacggttcttacactgagcttttcgctgctctttctcccaaggttactctcaaggagtctggtcgatacattggtccttggggtcagttccgagagcttcgagatgacgttcaggagggccttaccgatggtactgctcagaagctctgggattgggctgaggctgagattcgaccttacacttaaacgcgtgcaattaacagatagtttgccggtgataattctcttaacctcccacactcctttgacataacgatttatgtaacgaaactgaaatttgaccagatattgttgtaaatagaaaatctggcttgtaggtggcaaactagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacacctcgagcatgcatctagtggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgagg